(19) United States
(12) Patent Application Publication
(10) Pub. No.: US 2023/0374130 A1
(43) Pub. Date: Nov. 23, 2023

(54) BISPECIFIC ANTI LRRC15 AND CD3EPSILON ANTIBODIES

(71) Applicant: QLSF BIOTHERAPEUTICS, INC., South San Francisco, CA (US)

(72) Inventors: Aaron L. Kurtzman, South San Francisco, CA (US); Shihao Chen, San Mateo, CA (US); Mengyao Jin, South San Francisco, CA (US)

(21) Appl. No.: 17/588,086

(22) PCT Filed: Jul. 30, 2020
(86) PCT No.: PCT/US2020/070334

§ 371 Date: Jan. 28, 2022

Related U.S. Application Data
(60) Provisional application No. 62/880,347, filed on Jul. 30, 2019.

(51) Int. Cl. C07K 16/28; A61P 35/00

(52) U.S. Cl. CPC C07K 16/2809; C07K 16/28; A61P 35/00; A61K 2039/505

(57) ABSTRACT

Multispecific binding compound that bind to LRRC15 and CD3 are disclosed, along with methods of making such binding compounds, compositions, including pharmaceutical compositions, comprising such binding compounds, and their use to treat disorders that are characterized by the expression of LRRC15.

### CROSS-REFERENCE TO RELATED APPLICATIONS

This application claims priority benefit of the filing date of U.S. Provisional Patent Application No. 62/880,347, filed on Jul. 30, 2019, the disclosure of which application is incorporated by reference herein in its entirety.

### FIELD OF THE INVENTION

The present invention concerns multispecific binding compounds that bind to LRRC15 and CD3. The invention further concerns methods of making such binding compounds, compositions, including pharmaceutical compositions, comprising such binding compounds, and their use to treat disorders that are characterized by the expression of LRRC15.

### BACKGROUND OF THE INVENTION

### LRRC15

Leucine-rich repeats (LRRs) are 20- to 29-residue sequence motifs that are present in a number of proteins with diverse functions, such as hormone-receptor interactions, enzyme inhibition, cell adhesion and cellular trafficking. The primary function of these motifs appears to be to provide a versatile structural framework for the formation of protein-protein interactions. One protein that contains an LRR sequence motif is LRRC15, a leucine-rich transmembrane protein of 581 amino acids.

LRRC15 (Leucine Rich Repeat Containing 15, UniProt Q8TF66), also known as HLib and LIB, has been identified as being highly expressed in multiple solid tumor indications, with limited expression in normal tissue. Purcell et al., Cancer Res; 78(14); 4059-72. LRRC15 is a type 1 membrane protein with no obvious intracellular signaling domains. Id. This protein has been found to be highly expressed on the cell surface of stromal fibroblasts in many solid tumors. Id. Due to its limited expression in normal tissue, LRRC15 is an attractive target for the treatment of malignancies that are characterized by the expression of LRRC15. Monoclonal antibodies specific to LRRC15 have been described in the literature, e.g., U.S. Patent Publication No. US2017/0151343, the disclosure of which is incorporated by reference herein in its entirety.

RNA expression analysis demonstrates that LRRC15 is highly expressed in subsets of invasive breast carcinoma, colon adenocarcinoma, lymphoid neoplasm diffuse large B-cell lymphoma, esophageal carcinoma, head and neck squamous cell carcinoma, lung adenocarcinoma, lung squamous cell carcinoma, ovarian serous cystadenocarcinoma, pancreatic adenocarcinoma, and rectum adenocarcinoma. (http://gepia.cancer-pku.cn/detail.php?gene=LRRC15). LRRC15 expression in increased in smaller subsets of bladder urothelial carcinoma, cervical squamous cell carcinoma and endocervical adenocarcinoma, cholangio carcinoma, glioblastoma multiforme, sarcoma, skin cutaneous melanoma, stomach adenocarcinoma, testicular germ cell tumors, and uterine carcinosarcoma. Id.

LRRC15 is not over-expressed in adrenocortical carcinoma, kidney chromophobe, kidney renal clear cell carcinoma, kidney renal papillary cell carcinoma, acute myeloid leukemia, brain lower grade glioma, liver hepatocellular carcinoma, pheochromocytoma and paraganglioma, prostate adenocarcinoma, thyroid carcinoma, thymoma, or uterine corpus endometrial carcinoma. Id.

Purcell et al. reported histological analysis of LRRC15 expression. Notably, tumors from osteosarcoma, undifferentiated sarcoma, glioblastoma, and melanoma expressed LRRC15 directly as well as on the stroma surrounding the tumors. Interestingly, other tumors showed expression on the tumor-associated stroma, but not on the tumor cells themselves, e.g., pancreatic, breast, squamous lung, ovarian, testicular, gastric, head and neck, and colorectal cancers. LRRC15 expression was described on several cell lines, including U87 MG and U-118 MG (glioblastoma), RPMI-7951 and SKMEL2 (melanoma), and SAOS-2 (sarcoma).

In light of the above, therapeutic development of a multispecific binding compound (e.g., a bispecific antibody) may be efficacious in treating patients with various cancers that express LRRC15, in cancers surrounded by stroma wherein both the tumor cells and the stroma express LRRC15, and potentially in cancers that express LRRC15 only in the stroma surrounding the tumor.

### SUMMARY OF THE INVENTION

Aspects of the invention include multispecific binding compounds comprising a first binding unit having binding affinity to LRRC15, and a second binding unit having binding affinity to CD3ε, wherein the first binding unit comprises a heavy chain variable region having at least 95% sequence identity to any one of the sequences of SEQ ID NOs: 24-26 and/or a light chain variable region having at least 95% sequence identity to the sequence of SEQ ID NO: 27.

In some embodiments, the first binding unit comprises a heavy chain variable region sequence selected from the group consisting of SEQ ID NOs: 24-26 and/or a light chain variable region sequence of SEQ ID NO: 27. In some embodiments, the first binding unit comprises: a heavy chain variable region sequence of SEQ ID NO: 25 and a light chain variable region sequence of SEQ ID NO: 27. In some embodiments, the second binding unit comprises a heavy chain variable region having at least 95% sequence identity to any one of the sequences of SEQ ID NOs: 28-80 and/or a light chain variable region having at least 95% sequence identity to any one of the sequences of SEQ ID NOs: 81-132. In some embodiments, the second binding unit comprises a heavy chain variable region sequence selected from the group consisting of SEQ ID NOs: 28-80 and/or a light chain variable region sequence selected from the group consisting of SEQ ID NOs: 81-132. In some embodiments, the second binding unit comprises: (a) a heavy chain variable region sequence of SEQ ID NO: 55 and a light chain variable region sequence of SEQ ID NO: 107; or (b) a heavy chain variable region sequence of SEQ ID NO: 28 and a light chain variable region sequence of SEQ ID NO: 81; or (c) a heavy chain variable region sequence of SEQ ID NO: 29 and a light chain variable region sequence of SEQ ID NO: 82. In some embodiments, the second binding unit comprises a single chain Fv (scFv) comprising a first variable region sequence, a second variable region sequence, and a linker sequence that links the first variable region sequence to the second variable region sequence. In some embodiments, the linker sequence comprises the sequence of SEQ ID NO: 229.

Aspects of the invention include multispecific binding compounds comprising a first light chain polypeptide (L1), a first heavy chain polypeptide (H1), and a second heavy chain polypeptide (H2), wherein: L1 comprises a variable region sequence (L1V_{L}) and a constant region sequence (L1C_{L}); H1 comprises a variable region sequence (H1V_{H}), a C_{H}1 constant region sequence (H1C_{H}1), a C_{H}2 constant region sequence (H1C_{H}2), and a C_{H}3 constant region sequence (H1C_{H}3); and H2 comprises: a single chain Fv (H2scFv) comprising a first variable region sequence (H2scFv_{H}), a second variable region sequence (H2scFv_{L}), and a linker sequence that links the H2scFv_{H} sequence to the H2scFv_{L} sequence; a C_{H}2 constant region sequence (H2C_{H}2); and a C_{H}3 constant region sequence (H2C_{H}3); wherein: the L1V_{L} and H1V_{H} sequences together form a binding unit having binding affinity to LRRC15; the H2scFv has binding affinity to CD3ε; the L1C_{L} and H1C_{H}1 sequences are optionally connected by a disulfide bond; the H1 and H2 polypeptide chains optionally comprise a hinge region where the H1 and H2 polypeptide chains are optionally connected by at least one disulfide bond; and the H1C_{H}3 and H2C_{H}3 sequences comprise an asymmetric interface that facilitates proper pairing between the H1 and H2 polypeptide chains.

In some embodiments, the L1V_{L} sequence comprises a sequence having at least 95% sequence identity to the sequence of SEQ ID NO: 27. In some embodiments, the L1V_{L} sequence comprises the sequence of SEQ ID NO: 27. In some embodiments, the H1V_{H} sequence comprises a sequence having at least 95% sequence identity to any one of the sequences of SEQ ID NOs: 24-26. In some embodiments, the H1V_{H} sequence is selected from the group consisting of SEQ ID NOs: 24-26. In some embodiments, the H2scFv comprises a sequence having at least 95% sequence identity to any one of the sequences of SEQ ID NOs: 133-185. In some embodiments, the H2scFv comprises a sequence selected from the group consisting of SEQ ID NOs: 133-185.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises a variable region sequence (L1V_{L}) and a constant region sequence (L1C_{L}); H1 comprises a variable region sequence (H1V_{H}), a C_{H}1 constant region sequence (H1C_{H}1), a C_{H}2 constant region sequence (H1C_{H}2), a C_{H}3 constant region sequence (H1C_{H}3), and a single chain Fv (H1scFv) comprising a first variable region sequence (H1scFv_{H}), a second variable region sequence (H1scFv_{L}), and a linker sequence that links the H1scFv_{H} sequence to the H1scFv_{L} sequence; and H2 comprises a variable region sequence (H2V_{H}), a C_{H}1 constant region sequence (H2C_{H}1), a C_{H}2 constant region sequence (H2C_{H}2), a C_{H}3 constant region sequence (H2C_{H}3), and a single chain Fv (H2scFv) comprising a first variable region sequence (H2scFv_{H}), a second variable region sequence (H2scFv_{L}), and a linker sequence that links the H2scFv_{H} sequence to the H2scFv_{L} sequence; and L2 comprises a variable region sequence (L2V_{L}) and a constant region sequence (L2C_{L}); wherein: the L1V_{L} and H1V_{H} sequences together form a binding unit having binding affinity to LRRC15; the L2V_{L} and H2V_{H} sequences together form a binding unit having binding affinity to LRRC15; the H1scFv and the H2scFv have binding affinity to CD3ε; the L1C_{L} and H1C_{H}1 sequences are optionally connected by a disulfide bond; the L2C_{L} and H2C_{H}1 sequences are optionally connected by a disulfide bond; the H1 and H2 polypeptide chains optionally comprise a hinge region where the H1 and H2 polypeptide chains are optionally connected by at least one disulfide bond.

In some embodiments, L1 and L2 comprise identical sequences. In some embodiments, H1 and H2 comprise identical sequences. In some embodiments, L1 and L2 comprise different sequences. In some embodiments, H1 and H2 comprise different sequences. In some embodiments, the L1V_{L} sequence comprises a sequence having at least 95% sequence identity to the sequence of SEQ ID NO: 27. In some embodiments, the L1V_{L} sequence comprises the sequence of SEQ ID NO: 27. In some embodiments, the H1V_{H} sequence comprises a sequence having at least 95% sequence identity to any one of the sequences of SEQ ID NOs: 24-26. In some embodiments, the H1V_{H} sequence is selected from the group consisting of SEQ ID NOs: 24-26. In some embodiments, the H1scFv comprises a sequence having at least 95% sequence identity to any one of the sequences of SEQ ID NOs: 133-185. In some embodiments, the H1scFv comprises a sequence selected from the group consisting of SEQ ID NOs: 133-185. In some embodiments, the L2V_{L} sequence comprises a sequence having at least 95% sequence identity the sequence of SEQ ID NO: 27. In some embodiments, the L2V_{L} sequence comprises the sequence of SEQ ID NO: 27. In some embodiments, the H2V_{H} sequence comprises a sequence having at least 95% sequence identity to any one of the sequences of SEQ ID NOs: 24-26. In some embodiments, the H2V_{H} sequence is selected from the group consisting of SEQ ID NOs: 24-26. In some embodiments, the H2scFv comprises a sequence having at least 95% sequence identity to any one of the sequences of SEQ ID NOs: 133-185. In some embodiments, the H2scFv comprises a sequence selected from the group consisting of SEQ ID NOs: 133-185. In some embodiments, H1 comprises a sequence order, proceeding from an N-terminus to a C-terminus, as follows: H1V_{H}, H1C_{H}1, H1C_{H}2, H1C_{H}3, H1scFv. In some embodiments, H2 comprises a sequence order, proceeding from an N-terminus to a C-terminus, as follows: H2V_{H}, H2C_{H}1, H2C_{H}2, H2C_{H}3, H2scFv. In some embodiments, H1 comprises a sequence order, proceeding from an N-terminus to a C-terminus, as follows: H1V_{H}, H1C_{H}1, H1scFv, H1C_{H}2, H1C_{H}3. In some embodiments, H2 comprises a sequence order, proceeding from an N-terminus to a C-terminus, as follows: H2V_{H}, H2C_{H}1, H2scFv, H2C_{H}2, H2C_{H}3.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises a variable region sequence (L1V_{L}) and a constant region sequence (L1C_{L}); H1 comprises a variable region sequence (H1V_{H}), a C_{H}1 constant region sequence (H1C_{H}1), a C_{H}2 constant region sequence (H1C_{H}2), and a C_{H}3 constant region sequence (H1C_{H}3); H2 comprises a variable region sequence (H2V_{H}), a C_{H}1 constant region sequence (H2C_{H}1), a C_{H}2 constant region sequence (H2C_{H}2), a C_{H}3 constant region sequence (H2C_{H}3), and a single chain Fv (H2scFv) comprising a first variable region sequence (H2scFv_{H}), a second variable region sequence (H2scFv_{L}), and a linker sequence that links the H2scFv_{H} sequence to the H2scFv_{L} sequence; and L2 comprises a variable region sequence (L2V_{L}) and a constant region sequence (L2C_{L}); wherein: the L1V_{L} and H1V_{H} sequences together form a binding unit having binding affinity to LRRC15; the L2V_{L} and H2V_{H} sequences together form a binding unit having binding affinity to LRRC15; the H2scFv has binding affinity to CD3ε; the L1C_{L} and H1 C_{H}1 sequences are optionally connected by a disulfide bond; the L2C_{L} and H2C_{H}1 sequences are optionally connected by a disulfide bond; the H1 and H2 polypeptide chains optionally comprise a hinge region where the H1 and H2 polypeptide chains are optionally connected by at least one disulfide bond; and the H1C_{H}3 and H2C_{H}3 sequences comprise an asymmetric interface that facilitates proper pairing between the H1 and H2 polypeptide chains.

In some embodiments, L1 and L2 comprise identical sequences. In some embodiments, L1 and L2 comprise different sequences. In some embodiments, the L1V_{L} sequence comprises a sequence having at least 95% sequence identity to the sequence of SEQ ID NO: 27. In some embodiments, the L1V_{L} sequence comprises the sequence of SEQ ID NO: 27. In some embodiments, the H1V_{H} sequence comprises a sequence having at least 95% sequence identity to any one of the sequences of SEQ ID NOs: 24-26. In some embodiments, the H1V_{H} sequence is selected from the group consisting of SEQ ID NOs: 24-26. In some embodiments, the H2scFv comprises a sequence having at least 95% sequence identity to any one of the sequences of SEQ ID NOs: 133-185. In some embodiments, the H2scFv comprises a sequence selected from the group consisting of SEQ ID NOs: 133-185. In some embodiments, the L2V_{L} sequence comprises a sequence having at least 95% sequence identity to the sequence of SEQ ID NO: 27. In some embodiments, the L2V_{L} sequence comprises the sequence of SEQ ID NO: 27. In some embodiments, the H2V_{H} sequence comprises a sequence having at least 95% sequence identity to any one of the sequences of SEQ ID NOs: 24-26. In some embodiments, the H2V_{H} sequence is selected from the group consisting of SEQ ID NOs: 24-26. In some embodiments, H1 comprises a sequence order, proceeding from an N-terminus to a C-terminus, as follows: H1V_{H}, H1C_{H}1, H1C_{H}2, H1C_{H}3. In some embodiments, H2 comprises a sequence order, proceeding from an N-terminus to a C-terminus, as follows: H2V_{H}, H2C_{H}1, H2C_{H}2, H2C_{H}3, H2scFv. In some embodiments, H2 comprises a sequence order, proceeding from an N-terminus to a C-terminus, as follows: H2V_{H}, H2C_{H}1, H2scFv, H2C_{H}2, H2C_{H}3.

Aspects of the invention include multispecific binding compounds comprising a first light chain polypeptide (L1), a first heavy chain polypeptide (H1), and a second heavy chain polypeptide (H2), wherein: L1 comprises a sequence having at least 95% sequence identity to the sequence of SEQ ID NO: 194; H1 comprises a sequence having at least 95% sequence identity to the sequence of SEQ ID NO: 201; and H2 comprises a sequence having at least 95% sequence identity to any one of the sequences of SEQ ID NOs: 224 to 228, or 232.

Aspects of the invention include multispecific binding compounds comprising a first light chain polypeptide (L1), a first heavy chain polypeptide (H1), and a second heavy chain polypeptide (H2), wherein: L1 comprises the sequence of SEQ ID NO: 194; H1 comprises the sequence of SEQ ID NO: 201; and H2 comprises a sequence selected from the group consisting of SEQ ID NOs: 224 to 228, or 232.

Aspects of the invention include multispecific binding compounds comprising a first light chain polypeptide (L1), a first heavy chain polypeptide (H1), and a second heavy chain polypeptide (H2), wherein: L1 comprises SEQ ID NO: 194; H1 comprises SEQ ID NO: 201; and H2 comprises SEQ ID NO: 225.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises a sequence having at least 95% sequence identity to the sequences of SEQ ID NO: 194; H1 comprises a sequence having at least 95% sequence identity to any one of the sequences of SEQ ID NOs: 195-223, 231, 233-240; H2 comprises a sequence having at least 95% sequence identity to any one of the sequences of SEQ ID NOs: 195-223, 231, 233-240; and L2 comprises a sequence having at least 95% sequence identity to the sequence of SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises the sequence of SEQ ID NO: 194; H1 comprises a sequence selected from the group consisting of SEQ ID NOs: 195-223, 231, 233-240; H2 comprises a sequence selected from the group consisting of SEQ ID NOs: 195-223, 231, 233-240; and L2 comprises the sequence of SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 195 and 198; H2 comprises a sequence selected from the group consisting of SEQ ID NOs: 195 and 198; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 196 and 199; H2 comprises a sequence selected from the group consisting of SEQ ID NOs: 196 and 199; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 197 and 200; H2 comprises a sequence selected from the group consisting of SEQ ID NOs: 197 and 200; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 233 and 234; H2 comprises a sequence selected from the group consisting of SEQ ID NOs: 233 and 234; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises SEQ ID NO: 201; H2 comprises a sequence selected from the group consisting of SEQ ID NOs: 202, 206, 209, and 212; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises SEQ ID NO: 201; H2 comprises a sequence selected from the group consisting of SEQ ID NOs: 203, 207, 210, and 213; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises SEQ ID NO: 201; H2 comprises a sequence selected from the group consisting of SEQ ID NOs: 204, 208, 211, and 214; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises SEQ ID NO: 201; H2 comprises SEQ ID NO: 205; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises SEQ ID NO: 231; H2 comprises a sequence selected from the group consisting of SEQ ID NOs: 235, 236, and 237; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 215, 219, 221, and 239; H2 comprises a sequence selected from the group consisting of SEQ ID NOs: 215, 219, 221, and 239; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 216, 220, 222, and 240; H2 comprises a sequence selected from the group consisting of SEQ ID NOs: 216, 220, 222, and 240; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 217 and 223; H2 comprises a sequence selected from the group consisting of SEQ ID NOs: 217 and 223; and L2 comprises SEQ ID NO: 194.

Aspects of the invention include multispecific binding compounds comprising a first light chain polypeptide (L1), a first heavy chain polypeptide (H1), a second heavy chain polypeptide (H2), and a second light chain polypeptide (L2), wherein: L1 comprises SEQ ID NO: 194; H1 comprises SEQ ID NO: 218; H2 comprises SEQ ID NO: 218; and L2 comprises SEQ ID NO: 194.

Aspects of the invention include pharmaceutical compositions comprising the multispecific binding compounds described herein. Aspects of the invention include methods of treatment, comprising administering to an individual in need an effective dose of a multispecific binding compound or a pharmaceutical composition described herein. Aspects of the invention include methods for the treatment of a disorder characterized by expression of LRRC15, comprising administering to a subject with said disorder a multispecific binding compound or a pharmaceutical composition described herein.

Aspects of the invention include use of a multispecific binding compound as described herein in the preparation of a medicament for the treatment of a disorder characterized by expression of LRRC15. Aspects of the invention include multispecific binding compounds as described herein, for use in the treatment of a disorder characterized by expression of LRRC15.

In some embodiments, the disorder is selected from the group consisting of: sarcoma, breast cancer, lung cancer, ovarian cancer, pancreatic cancer, and large B cell lymphoma.

Aspects of the invention include polynucleotidse encoding a multispecific binding compound as described herein, a vector comprising a polynucleotide as described herein, and a cell comprising a vector as described herein.

Aspects of the invention include methodd of producing a multispecific binding compound as described herein, comprising growing a cell as described herein under conditions permissive for expression of the multispecific binding compound, and isolating the multispecific binding compound.

These and further aspects will be further explained in the rest of the disclosure, including the Examples.

### BRIEF DESCRIPTION OF THE DRAWINGS

FIG. 1 is a table showing percent aggregate, percent monomer, and melting point for multispecific binding compounds in accordance with embodiments of the invention.

FIG. 2 is a table showing binding kinetics of multispecific binding compounds in accordance with embodiments of the invention.

FIG. 3, Panels A-F, are schematic illustrations of various multispecific binding compounds in accordance with embodiments of the invention.

FIG. 4 is a graph showing results of a protein thermal shift assay conducted on various multispecific binding compounds in accordance with embodiments of the invention.

FIG. 5, Panels A and B, are graphs showings binding as a function of concentration for anti-CD3 scFv-Fc clones binding to human Jurkat cells (Panel A) and to Cynomologous H-SCF cells (Panel B).

FIG. 6, Panels A-F, are graphs showing binding of various bispecific binding compound formats to CD3+ cells. Panel A shows binding of scFv-Fc compounds to CD3+ cells. Panel B shows binding of Type 1 compounds to CD3+ cells. Panel C shows binding of Type 2 compounds to CD3+ cells. Panels D, E, and F compares binding of Type 4 compounds, Type 5 compounds, and scFv-Fc compounds to CD3+, CD4+ T cells and to CD3+, CD8+ T cells.

FIG. 7, Panels A and B, are graphs showing binding to LRRC15+U118MG and U87MG cells, respectively, as a function of concentration for various binding compound formats.

FIG. 8, Panels A-E, are graphs showing T-cell activation as a function of concentration for various binding compound formats.

FIG. 9, Panels A-E, are graphs showing proliferation of T-cells as a function of concentration for various binding compound formats

FIG. 10, Panels A-C, are graphs showing cytokine release as a function of concentration for various binding compound formats.

FIG. 11, Panels A-G, are graphs showing percent cytotoxicity as a function of concentration for various binding compound formats.

FIG. 12 is a graph showing tumor volume as a function of time for animals dosed with various binding compound formats or controls.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

Antibodies, also referred to as immunoglobulins, conventionally comprise at least one heavy chain and one light chain, where the amino terminal domain of the heavy and light chains is variable in sequence, hence is commonly referred to as a variable region domain, or a variable heavy (VH) or variable light (VH) domain. The two domains conventionally associate to form a specific binding region, although as will be discussed here, specific binding can also be obtained with heavy chain-only variable sequences, and a variety of non-natural configurations of antibodies are known and used in the art.

A “functional” or “biologically active” antibody or binding compound is one capable of exerting one or more activities in structural, regulatory, biochemical or biophysical events. For example, a functional antibody or other binding compound may have the ability to specifically bind an antigen and the binding may in turn elicit or alter a cellular or molecular event such as signal transduction or enzymatic activity. A functional antibody or other binding compound may also block ligand activation of a receptor or act as an agonist or antagonist. The capability of an antibody or other binding compound to exert one or more activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), three chain antibodies, single chain Fv (scFv), nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, a cancer cell, or cells that produce autoimmune antibodies associated with an autoimmune disease. The immunoglobulins disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. The immunoglobulins can be derived from any species.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present invention can be made by the hybridoma method first described by Kohler et al. (1975) Nature 256:495, and can also be made via recombinant protein production methods (see, e.g., U.S. Pat. No. 4,816,567), for example.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” residues 26-32 (H1), 53-55 (H2) and 96-101 (H3) in the heavy chain variable domain; Chothia and Lesk J Mol. Biol. 196:901-917 (1987)). “Framework Region” or “FR” residues are those variable domain residues other than the hypervariable region residues as herein defined.

Exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010;47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001;309:657-670; Ofran et al. “Automated identification of complementarity determining regions (CDRs) reveals peculiar characteristics of CDRs and B cell epitopes.” J Immunol. 2008;181:6230-6235; Almagro “Identification of differences in the specificity-determining residues of antibodies that recognize antigens of different size: implications for the rational design of antibody repertoires.” J Mol Recognit. 2004;17:132-143; and Padlanet al. “Identification of specificity-determining residues in antibodies.” Faseb J. 1995;9:133-139., each of which is herein specifically incorporated by reference.

The term “multispecific binding compound” as used herein means a binding compound that comprises two or more antigen binding sites. Multispecific binding compounds in accordance with embodiments of the invention can be antibody-like molecules comprising, consisting essentially of, or consisting of two, three, or four polypeptide subunits, any of which may comprise one or more variable region domains having binding affinity for a target antigen (e.g., LRRC15). In some embodiments, a multispecific binding compound comprises pair of variable region domains (e.g., a heavy chain variable region domain and a light chain variable region domain) that together form a binding unit. In some embodiments, a multispecific binding compound comprises a pair of variable region domains in a single chain Fv (scFv) format, wherein a first variable region domain and a second variable region domain are connected by a linker, and together form a binding unit. The subject multispecific binding compounds can have any suitable combination or configuration of binding units, including but not limited to the specific configurations described herein.

Multispecific binding compounds as described herein may belong to any immunoglobulin subclass, including IgG, IgM, IgA, IgD and IgE subclasses. In a particular embodiment, the multispecific binding compound is of the IgG1, IgG2, IgG3, or IgG4 subtype, in particular the IgG1 subtype. Modifications of CH domains that alter effector function are further described herein.

An “intact antibody chain” as used herein is one comprising a full length variable region and a full length constant region (Fc). An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called κ and λ, based on the amino acid sequences of their constant domains.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIIB1; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The human IgG1 amino acid sequence is provided by UniProtKB No. P01857, which is incorporated by reference herein in its entirety. The human IgG2 amino acid sequence is provided by UniProtKB No. P01859, which is incorporated by reference herein in its entirety. The human IgG3 amino acid sequence is provided by UniProtKB No. P01860, which is incorporated by reference herein in its entirety. The human IgG4 amino acid sequence is provided by UniProtKB No. P01861, which is incorporated by reference herein in its entirety.

Variant Fc sequences may include three amino acid substitutions in the CH2 region to reduce FcγRI binding at EU index positions 234, 235, and 237 (see Duncan et al., (1988) Nature 332:563; Hezareh et al., (2001) J. Virology 75:12161; U.S. Pat. No.5,624,821, the disclosures of which are incorporated herein by reference in their entireties). In some embodiments, a variant Fc sequence can include the following amino acid substitutions: L234A; L235A; and G237A. When these three amino acid substitutions are present in an IgG1 Fc sequence, they can be referred to as G1AAA or LALAGA.

Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour KL. et al., 1999 Eur J Immunol. 29(8):2613-24; and Shields RL. et al., 2001. J Biol Chem. 276(9):6591-604).

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of a binding compound can comprise one or more mutations in the hinge region to eliminate disulfide bonding. In yet another embodiment, the hinge region of an Fc can be removed entirely. In still another embodiment, a binding compound can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, an antibody having an Fc region according to this invention can comprise an antibody with or without K447.

Aspects of the invention include binding compounds having multi-specific configurations, which include, without limitation, bispecific, trispecific, etc. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 192) (n=1) and GGGGSGGGGS (SEQ ID NO: 193) (n=2). Other suitable linkers can also be used, and are described, for example, in Chen et al., Adv Drug Deliv Rev. 2013 Oct. 15; 65(10): 1357-69, the disclosure of which is incorporated herein by reference in its entirety.

Antibodies and multispecific binding compounds as described herein can be in the form of a dimer, in which two heavy chains are disulfide bonded or otherwise covalently or non-covalently attached to each other, and can optionally include an asymmetric interface between two or more of the CH domains to facilitate proper pairing between polypeptide chains (commonly referred to as a “knobs-into-holes” interface). Knobs into holes antibody engineering techniques for heavy chain heterodimerization are discussed, for example, in Ridgway et al., Protein Eng. 1996 July;9(7):17-21, and U.S. Pat. No. 8,216,805, the disclosures of which are incorporated by reference herein in their entireties. An Fc region comprising an asymmetric interface can be referred to herein with the abbreviation “Kill”, meaning knobs-into-holes. For example, aspects of the invention include a variant Fc region sequence, such as a G1AAA sequence, that contains an asymmetric interface, and which is referred to herein as “G1AAA KiH”.

The terms “LRRC15” and “Leucine Rich Repeat Containing 15” refer to an LRRC15 protein of any human and non-human animal species, and specifically includes human LRRC15 as well as LRRC15 of non-human mammals.

The term “human LRRC15” as used herein includes any variants, isoforms and species homologs of human LRRC15 (UniProt Q8TF66), regardless of its source or mode of preparation. Thus, “human LRRC15” includes human LRRC15 naturally expressed by cells and LRRC15 expressed on cells transfected with the human LRRC15 gene.

The terms “anti-LRRC15 antibody,” “LRRC15 antibody,” “anti-LRRC15 binding compound” and “LRRC15 binding compound” are used herein interchangeably to refer to an antibody or binding compound as herein defined, immunospecifically binding to LRRC15, including human LRRC15, as herein defined.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody or binding compound is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Binding compounds of the invention include multi-specific binding compounds. Multi-specific binding compounds have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. The terms “multi-specific antibody” and “multi-specific binding compound” are used herein in the broadest sense and cover all antibodies and antibody-like molecules with more than one binding specificity. The multi-specific anti-LRRCS binding compounds of the present invention specifically include binding compounds immunospecifically binding to an epitope on an LRRC15 protein, such as a human LRRC15, and to an epitope on a different protein, such as, for example, a CD3 protein.

An “epitope” is the site on the surface of an antigen molecule to which a single antibody molecule binds. Generally, an antigen has several or many different epitopes and reacts with many different antibodies. The term specifically includes linear epitopes and conformational epitopes.

Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

The term “valent” as used herein refers to a specified number of binding sites in an antibody molecule or binding compound.

A “monovalent” binding compound has one binding site. Thus a monovalent binding compound is also monospecific.

A “multi-valent” binding compound has two or more binding sites. Thus, the terms “bivalent”, “trivalent”, and “tetravalent” refer to the presence of two binding sites, three binding sites, and four binding sites, respectively. Thus, a bispecific binding compound according to the invention is at least bivalent and may be trivalent, tetravalent, or otherwise multi-valent. A bivalent binding compound in accordance with embodiments of the invention may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB) and binding compounds, tri-specific antibodies and binding compounds, and the like.

The term “human antibody” is used herein to include antibodies having variable and constant regions derived from human germline immunoglobulin sequences. The human antibodies herein may include amino acid residues not encoded by human germline immunoglobulin sequences, e.g., mutations introduced by random or site-specific mutagenesis in vitro or by somatic mutation in vivo. The term “human antibody” specifically includes antibodies and binding compounds having human heavy chain variable region sequences.

The term “chimeric” antibody as used herein refers to an antibody having variable sequences derived from a non-human immunoglobulin, such as a rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397, which are incorporated herein by reference in their entireties. The term “chimeric antibody” specifically includes antibodies and binding compounds having variable region sequences derived from a non-human immunoglobulin, and human immunoglobulin constant region sequences.

The term “humanized antibody” as used herein refers to an antibody or binding compound that contains minimal sequences derived from a non-human immunoglobulin. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin sequence. A humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are incorporated herein by reference in their entireties.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T cell receptors or FcRs and perform effector functions. Preferably, the cells express at least FcγRIII and perform ADCC effector function. Examples of human leukocytes which mediate ADCC include natural killer (NK) cells, monocytes, cytotoxic T cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B cells and T cells including cytolytic T cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al., J. Immunol. Methods 202:163 (1996), may be performed.

“Directed T-cell mediated cytotoxicity” and “re-directed T-cell mediated cytotoxicity”, as used interchangeably herein, refer to a cell-mediated reaction in which a cross-linking molecule (e.g., a bispecific antibody) crosslinks a surface antigen on a T-cell (e.g., CD3) and an antigen on a target cell (e.g., a surface antigen on a cancer cell). Crosslinking of the T-cell and the target cell facilitates killing of the target cell by the T-cell via cytotoxic activity of the T-cell. Re-directed T-cell mediated cytotoxicity is described, for example, in Velasquez et al., Blood 2018 131: 30-38.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (KD). Affinity can be measured by common methods known in the art. Low-affinity antibodies generally bind antigen slowly and tend to dissociate readily, whereas high-affinity antibodies generally bind antigen faster and tend to remain bound.

As used herein, the “KD” or “KD value” refers to a dissociation constant determined by BioLayer Interferometry, using an Octet Red96 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The KD is the ratio of koff/kon. (For further details see, Concepcion, J, et al., Comb Chem High Throughput Screen, 12(8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), skin cancer, melanoma, lung cancer, including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer (e.g., pancreatic ductal adenocarcinoma), glioblastoma, cervical cancer, ovarian cancer (e.g., high grade serous ovarian carcinoma), liver cancer (e.g., hepatocellular carcinoma (HCC)), bladder cancer (e.g., urothelial bladder cancer), testicular (germ cell tumor) cancer, hepatoma, breast cancer, brain cancer (e.g., astrocytoma), colon cancer, rectal cancer, colorectal cancer, endometrial or uterine carcinoma, salivary gland carcinoma, kidney or renal cancer (e.g., renal cell carcinoma, nephroblastoma or Wilms' tumour), prostate cancer, vulval cancer, thyroid cancer, hepatic carcinoma, anal carcinoma, penile carcinoma, as well as head and neck cancer. Additional examples of cancer include, without limitation, retinoblastoma, thecomas, arrhenoblastomas, hepatoma, hematologic malignancies including non-Hodgkin's lymphoma (NHL), multiple myeloma and acute hematologic malignancies, endometrial or uterine carcinoma, endometriosis, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, and urinary tract carcinomas.

The term “metastatic cancer” means the state of cancer where the cancer cells of a tissue of origin are transmitted from the original site to one or more sites elsewhere in the body, by the blood vessels or lymphatics, to form one or more secondary tumors in one or more organs besides the tissue of origin. A prominent example is metastatic breast cancer.

The term “characterized by expression of LRRC15” broadly refers to any disease or disorder in which LRRC15 expression is associated with or involved with one or more pathological processes that are characteristic of the disease or disorder. Specifically, and without limitation, a disease or disorder that is characterized by expression of LRRC15 includes, e.g., a cancer in which tumor cells express LRRC15, and/or tumor-associated stroma exhibits expression of LRRC15. Such disorders include, but are not limited to: invasive breast carcinoma, colon adenocarcinoma, lymphoid neoplasm diffuse large B-cell lymphoma, esophageal carcinoma, head and neck squamous cell carcinoma, lung adenocarcinoma, lung squamous cell carcinoma, ovarian serous cystadenocarcinoma, pancreatic adenocarcinoma, rectum adenocarcinoma, bladder urothelial carcinoma, cervical squamous cell carcinoma and endocervical adenocarcinoma, cholangio carcinoma, glioblastoma multiforme, sarcoma (e.g., undifferentiated sarcoma), skin cutaneous melanoma, stomach adenocarcinoma, testicular germ cell tumors, uterine carcinosarcoma, osteosarcoma, glioblastoma, melanoma, ovarian, gastric, and colorectal cancers.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

The terms “treat”, “treatment” or “treating” as used herein refer to both therapeutic treatment and prophylactic of preventative measures, wherein the object is to prevent or slow down (lessen) a targeted pathological condition or disorder. A subject in need of treatment includes a subject already having a particular condition or disorder, as well as a subject prone to having the disorder or a subject in whom the disorder is to be prevented.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomeriation), clipping/hydrolysis/fragmentation (e.g., hinge region fragmentation), succinimide formation, unpaired cysteine(s), N-terminal extension, C-terminal processing, glycosylation differences, etc.

### II. Detailed Description

### Anti-LRRC15 Binding Compounds

Aspects of the invention include multispecific binding compounds that have binding affinity for LRRC15 and for CD3ε. The multispecific binding compounds can comprise various configurations, and each binding unit can comprise a set of CDR sequences. CDR sequences are provided in Tables 1 and 2. Anti-LRRC15 heavy chain CDR sequences include SEQ ID NOs: 1, 3, and 13, and anti-LRRC15 light chain CDR sequences include SEQ ID NOs: 15, 19 and 22. Anti-CD3ε heavy chain CDR sequences include SEQ ID NOs: 2, 4-12, and 14, and anti-CD3ε light chain CDR sequences include SEQ ID NOs: 16-18, 20-21, and 23. In some embodiments, a multispecific binding compound comprises a CDR sequence with two or fewer amino acid substitutions in any one SEQ ID NOs: 1-23.

Multispecific binding compounds in accordance with embodiments of the invention can comprise any suitable combination of heavy chain and light chain variable region sequences, as listed in Tables 3, 4, 5 and 6. Anti- LRRC15 heavy chain variable region sequences include SEQ ID NOs: 24-26. Anti-LRRC15 light chain variable region sequences include SEQ ID NO: 27. Anti-CD3ε heavy chain variable region sequences include SEQ ID NOs: 28-80. Anti-CD3ε light chain variable region sequences include SEQ ID NOs: 81-132. In some embodiments, a multispecific binding compound comprises a variable region sequence having at least about 80% identity, such as about 85%, about 90%, about 95%, about 99%, or about 99.9% identity to a variable region sequence of any one of SEQ ID NOs: 24-132.

Multispecific binding compounds in accordance with embodiments of the invention can comprise one or more anti-CD3ε scFv sequences, as listed in Table 7. Anti-CD3ε scFv sequences include SEQ ID NOs: 133-185. In some embodiments, a multispecific binding compound comprises an scFv sequence having at least about 80% identity, such as about 85%, about 90%, about 95%, about 99%, or about 99.9% identity to an scFv sequence of any one of SEQ ID NOs: 133-185.

The multispecific binding compounds described herein provide a number of benefits that contribute to utility as clinically therapeutic agent(s). The multispecific binding compounds include members with a variety of binding unit configurations, allowing the selection of a specific molecule that shows therapeutic benefits.

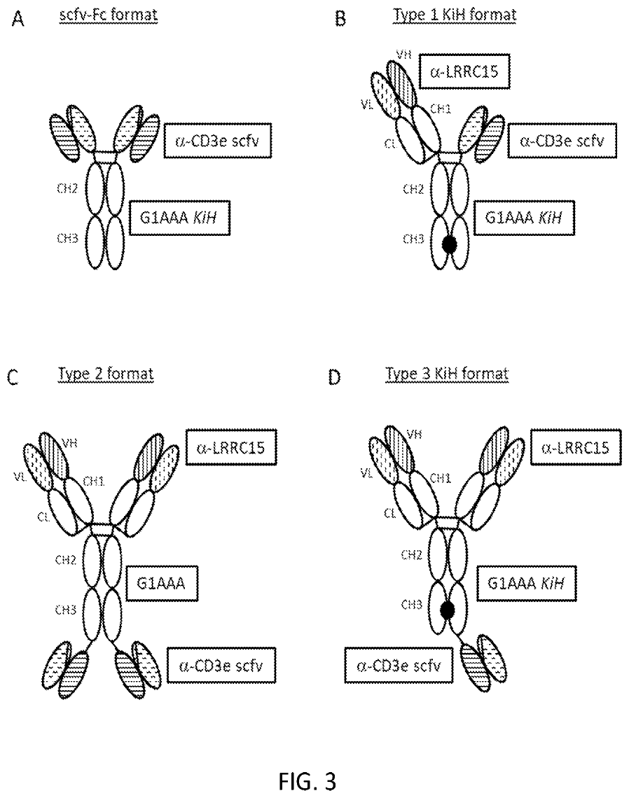

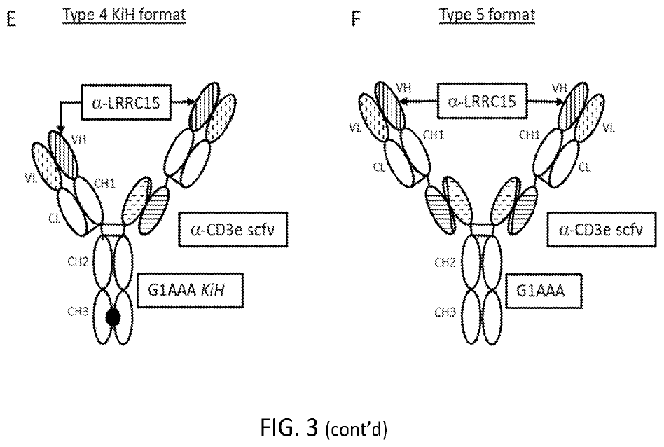

A suitable binding compound may be selected from those provided herein for development and therapeutic or other use, including, without limitation, use as a bispecific binding compound, e.g., as shown in FIG. 3, Panels A-F. FIG. 3 provides schematic illustrations of non-limiting examples of multispecific binding compounds in accordance with embodiments of the invention. In some embodiments, two heavy chains are paired using, e.g., knobs-into-holes technology.

Turning to the binding compounds depicted in FIG. 3, Panel A depicts an scFv-Fc format molecule comprising two heavy chains, wherein each heavy chain comprises an scFv with binding affinity to CD3ε, a hinge region, and an Fc region.

FIG. 3, Panel B depicts an asymmetric, bispecific binding compound comprising a first light chain, a first heavy chain, and a second heavy chain. The first light chain comprises a variable region (L1V_{L}) and constant region L1C_{L}). The first heavy chain comprises a variable region H1V_{H} and a constant region comprising a hinge region and C_{H}1, C_{H}2, and C_{H}3 domains. Together, the L1V_{L} and H1V_{H} regions for a binding unit that has binding affinity to LRRC15. The second heavy chain comprises an scFv with binding affinity to CD3ε, a hinge region, and C_{H}2 and C_{H}3 domains. The C_{H}2 and C_{H}3 domains constitute the Fc region. In some embodiments, a binding compound comprises an asymmetric interface between the C_{H}2 domains and/or the C_{H}3 domains of the first and second heavy chains, which ensures proper pairing between the first and second heavy chains. The binding compound depicted in FIG. 3, Panel B is referred to herein as a Type 1 binding compound.

FIG. 3, Panel C depicts a symmetric, bispecific binding compound that comprises a first light chain, a first heavy chain, a second heavy chain, and a second light chain The first light chain comprises a variable region (L1V_{L}) and constant region (L1C_{L}). The first heavy chain comprises a variable region H1V_{H}, a constant region comprising a hinge region and C_{H}1, C_{H}2, and C_{H}3 domains, and an scFv with binding affinity to CD3ε. Together, the L1V_{L} and H1V_{H} regions form a binding unit that has binding affinity to LRRC15. The second heavy chain comprises a variable region H2V_{H}, a constant region comprising a hinge region and C_{H}1, C_{H}2, and C_{H}3 domains, and an scFv having binding affinity to CD3ε. The C_{H}2 and C_{H}3 domains constitute the Fc region. The second light chain comprises a variable region (L2V_{L}) and constant region (L2C_{L}). Together, the L2V_{L} and H2V_{H} regions form a binding unit that has binding affinity to LRRC15. The binding compound depicted in FIG. 3, Panel C is referred to herein as a Type 2 binding compound.

FIG. 3, Panel D depicts an asymmetric, bispecific binding compound comprising a first light chain, a first heavy chain, a second heavy chain, and a second light chain The first light chain comprises a variable region (L1V_{L}) and constant region (L1C_{L}). The first heavy chain comprises a variable region H1V_{H} and a constant region comprising a hinge region and C_{H}1, C_{H}2, and C_{H}3 domains. Together, the L1V_{L} and H1V_{H} regions form a binding unit that has binding affinity to LRRC15. The second heavy chain comprises a variable region H2V_{H}, a hinge region, C_{H}1, C_{H}2 and C_{H}3 domains, and an scFv having binding affinity to CD3ε. The C_{H}2 and C_{H}3 domains constitute the Fc region. In some embodiments, a binding compound comprises an asymmetric interface between the C_{H}2 domains and/or the C_{H}3 domains of the first and second heavy chains, which ensures proper pairing between the first and second heavy chains. The second light chain comprises a variable region (L2V_{L}) and constant region (L2C_{L}). Together, the L2V_{L} and H2V_{H} regions form a binding unit that has binding affinity to LRRC15. The binding compound depicted in FIG. 3, Panel D is referred to herein as a Type 3 binding compound.

FIG. 3, Panel E depicts an asymmetric, bispecific binding compound comprising a first light chain, a first heavy chain, a second heavy chain, and a second light chain The first light chain comprises a variable region (L1V_{L}) and constant region (L1C_{L}). The first heavy chain comprises a variable region H1V_{H} and a constant region comprising a hinge region and C_{H}1, C_{H}2, and C_{H}3 domains. Together, the L1V_{L} and H1V_{H} regions form a binding unit that has binding affinity to LRRC15. The second heavy chain comprises a variable region H2V_{H}, a C_{H}1 domain, a first hinge region and/or a first linker region, an scFv having binding affinity to CD3ε, a second hinge region, and C_{H}2 and C_{H}3 domains. The C_{H}2 and C_{H}3 domains constitute the Fc region. In some embodiments, a binding compound comprises an asymmetric interface between the C_{H}2 domains and/or the C_{H}3 domains of the first and second heavy chains, which ensures proper pairing between the first and second heavy chains. The second light chain comprises a variable region (L2V_{L}) and constant region (L2C_{L}). Together, the L2V_{L} and H2V_{H} regions form a binding unit that has binding affinity to LRRC15. The binding compound depicted in FIG. 3, Panel E is referred to herein as a Type 4 binding compound.

FIG. 3, Panel F depicts a symmetric, bispecific binding compound that comprises a first light chain, a first heavy chain, a second heavy chain, and a second light chain The first light chain comprises a variable region (L1V_{L}) and constant region (L1C_{L}). The first heavy chain comprises a variable region H1V_{H}, a C_{H}1 domain, a first hinge region and/or a first linker region, an scFv having binding affinity to CD3ε, a second hinge region, and C_{H}2, and C_{H}3 domains. Together, the L1V_{L} and H1V_{H} regions form a binding unit that has binding affinity to LRRC15. The second heavy chain comprises a variable region H2V_{H}, a C_{H}1 domain, a first hinge region and/or a first linker region, an scFv having binding affinity to CD3ε, a second hinge region, and C_{H}2 and C_{H}3 domains. The C_{H}2 and C_{H}3 domains constitute the Fc region. The second light chain comprises a variable region (L2V_{L}) and constant region (L2C_{L}). Together, the L2V_{L} and H2V_{H} regions form a binding unit that has binding affinity to LRRC15. The binding compound depicted in FIG. 3, Panel F is referred to herein as a Type 5 binding compound.

In one preferred embodiment, a multispecific binding compound has binding affinity to LRRC15 and CD3ε, and comprises a first light chain polypeptide comprising the sequence of SEQ ID NO:194, a first heavy chain polypeptide comprising the sequence of SEQ ID NO:201, and a second heavy chain polypeptide comprising the sequence of SEQ ID NO:224.

In one preferred embodiment, a multispecific binding compound has binding affinity to LRRC15 and CD3ε, and comprises a first light chain polypeptide comprising the sequence of SEQ ID NO:194, a first heavy chain polypeptide comprising the sequence of SEQ ID NO:201, and a second heavy chain polypeptide comprising the sequence of SEQ ID NO:225.

In one preferred embodiment, a multispecific binding compound has binding affinity to LRRC15 and CD3ε, and comprises a first light chain polypeptide comprising the sequence of SEQ ID NO:194, a first heavy chain polypeptide comprising the sequence of SEQ ID NO:201, and a second heavy chain polypeptide comprising the sequence of SEQ ID NO:226.

In one preferred embodiment, a multispecific binding compound has binding affinity to LRRC15 and CD3ε, and comprises a first light chain polypeptide comprising the sequence of SEQ ID NO:194, a first heavy chain polypeptide comprising the sequence of SEQ ID NO:201, and a second heavy chain polypeptide comprising the sequence of SEQ ID NO:227.

In one preferred embodiment, a multispecific binding compound has binding affinity to LRRC15 and CD3ε, and comprises a first light chain polypeptide comprising the sequence of SEQ ID NO:194, a first heavy chain polypeptide comprising the sequence of SEQ ID NO:201, and a second heavy chain polypeptide comprising the sequence of SEQ ID NO:228.

In one preferred embodiment, a multispecific binding compound has binding affinity to LRRC15 and CD3ε, and comprises a first light chain polypeptide comprising the sequence of SEQ ID NO:194, a first heavy chain polypeptide comprising the sequence of SEQ ID NO:201, and a second heavy chain polypeptide comprising the sequence of SEQ ID NO:232.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises a sequence selected from the group consisting of SEQ ID NOs: 195 and 198; H2 comprises a sequence selected from the group consisting of SEQ ID NOs: 195 and 198; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises a sequence selected from the group consisting of SEQ ID NOs: 196 and 199; H2 comprises a sequence selected from the group consisting of SEQ ID NOs: 196 and 199; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 197 and 200; H2 comprises a sequence selected from the group consisting of SEQ ID NOs: 197 and 200; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein: L1 comprises SEQ ID NO: 194; H1 comprises a sequence selected from the group consisting of SEQ ID NOs: 233 and 234; H2 comprises a sequence selected from the group consisting of SEQ ID NOs: 233 and 234; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein: L1 comprises SEQ ID NO: 194; H1 comprises SEQ ID NO: 201; H2 comprises a sequence selected from the group consisting of SEQ ID NOs: 202, 206, 209, and 212; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises SEQ ID NO: 201; H2 comprises a sequence selected from the group consisting of SEQ ID NOs: 203, 207, 210, and 213; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises SEQ ID NO: 201; H2 comprises a sequence selected from the group consisting of SEQ ID NOs: 204, 208, 211, and 214; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises SEQ ID NO: 201; H2 comprises SEQ ID NO: 205; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises SEQ ID NO: 231; H2 comprises a sequence selected from the group consisting of SEQ ID NOs: 235, 236, and 237; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises a sequence selected from the group consisting of SEQ ID NOs: 215, 219, 221, and 239; H2 comprises a sequence selected from the group consisting of SEQ ID NOs: 215, 219, 221, and 239; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises a sequence selected from the group consisting of SEQ ID NOs: 216, 220, 222, and 240; H2 comprises a sequence selected from the group consisting of SEQ ID NOs: 216, 220, 222, and 240; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises a sequence selected from the group consisting of SEQ ID NOs: 217 and 223; H2 comprises a sequence selected from the group consisting of SEQ ID NOs: 217 and 223; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises SEQ ID NO: 218; H2 comprises SEQ ID NO: 218; and L2 comprises SEQ ID NO: 194.

In one preferred embodiment, a multispecific binding compound has binding affinity to LRRC15 and CD3ε, and comprises a first light chain polypeptide (L1), a first heavy chain polypeptide (H1), a second heavy chain polypeptide (H2), and a second light chain polypeptide (L2), wherein L1 comprises SEQ ID NO: 194; H1 comprises SEQ ID NO: 238; H2 comprises SEQ ID NO: 238; and L2 comprises SEQ ID NO: 194.

Determination of affinity for a candidate protein can be performed using methods known in the art, such as Biacore measurements. Multispecific binding compounds as described herein may have an affinity for LRRC15 or CD3ε with a Kd of from about 10⁻⁶ to around about 10⁻¹¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating an LRRC15 or CD3ε biological activity, including in vitro assays, pre-clinical models, and clinical trials, as well as assessment of potential toxicity.

Various formats of multispecific binding compounds are within the ambit of the invention, including, without limitation, two chain polypeptides, three chain polypeptides, and four chain polypeptides, as described herein. The multispecific binding compounds herein specifically include bispecific binding compounds having binding affinity to LRRC15 and CD3ε (e.g., anti-LRRC15 x anti-CD3ε binding compounds). Such bispecific binding compounds induce potent T-cell mediated killing of cells expressing LRRC15 and/or tumor cell associate stroma expressing LRRC15. Sequence information is provided in Tables 1-14.

TABLE 1
Heavy Chain CDR Sequences

| Clone ID | CDRH1 | CDRH2 | CDRH3 |
|---|---|---|---|
| huLRRC15_VHv2 | SYWIE (SEQ ID NO: 1) | EILPGSDTTNYNEKFKD | DRGNYRAWFGY |
| | | (SEQ ID NO: 3) | (SEQ ID NO: 13) |
| 1B4_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 1B10_HC | TYAMN (SEQ ID NO: 2) | RIRSKANNYATYYADSVKG | HGNFGNSYVSWFAY |
| | | (SEQ ID NO: 5) | (SEQ ID NO: 14) |
| 4A4_HC | TYAMN (SEQ ID NO: 2) | RIRSKYNNYATYYADPVKG | HGNFGNSYVSWFAY |
| | | (SEQ ID NO: 6) | (SEQ ID NO: 14) |
| 4A5_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4A9_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 4A10_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4A10.1_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4A11_HC | TYAMN (SEQ ID NO: 2) | RIRSKYNNYATYYAASVKD | HGNFGNSYVSWFAY |
| | | (SEQ ID NO: 8) | (SEQ ID NO: 14) |
| 4A11.1_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4A12_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4A12.1_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4B9_HC | TYAMN (SEQ ID NO: 2) | RIRSKANNYATYYADSVKG | HGNFGNSYVSWFAY |
| | | (SEQ ID NO: 5) | (SEQ ID NO: 14) |
| 4B9.1_HC | TYAMN (SEQ ID NO: 2) | RIRSKANNYATYYADSVKG | HGNFGNSYVSWFAY |
| | | (SEQ ID NO: 5) | (SEQ ID NO: 14) |
| 4B12_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4B12.1_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4C7_HC | TYAMN (SEQ ID NO: 2) | RIRSKTNNYATYYADSVKD | HGNFGNSYVSWFAY |
| | | (SEQ ID NO: 9) | (SEQ ID NO: 14) |
| 4C12_HC | TYAMN (SEQ ID NO: 2) | RIRSKSNNYATYYAASVKD | HGNFGNSYVSWFAY |
| | | (SEQ ID NO: 10) | (SEQ ID NO: 14) |
| 4D4_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 4D6_HC | TYAMN (SEQ ID NO: 2) | RIRSKSNNYATYYADSVKD | HGNFGNSYVSWFAY |
| | | (SEQ ID NO: 11) | (SEQ ID NO: 14) |
| 4D6.1_HC | TYAMN (SEQ ID NO: 2) | RIRSKSNNYATYYADSVKD | HGNFGNSYVSWFAY |
| | | (SEQ ID NO: 11) | (SEQ ID NO: 14) |
| 4D7_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4D9_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 4E7_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4F5_HC | TYAMN (SEQ ID NO: 2) | RIRSKYNNYATYYADPVKG | HGNFGNSYVSWFAY |
| | | (SEQ ID NO: 6) | (SEQ ID NO: 14) |
| 4F11_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 4G1_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 4G2_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 160C9_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 160D10_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 160F1_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 160H4_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 161A8_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 161B4_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 161E6_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 161F8_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 161G1_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 161H2_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 162B10_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 162B12_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 162D3_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 162D11_HC | TYAMN (SEQ ID NO: 2) | RIRSKYNNYATYYADPVKG | HGNFGNSYVSWFAY |
| | | (SEQ ID NO: 6) | (SEQ ID NO: 14) |
| 162G12_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 160D12_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 160E2_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 160E12_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 160F2_HC | TYAMN (SEQ ID NO: 2) | RIRSKYNNYATYYADPVKD | HGNFGNSYVSWFAY |
| | | (SEQ ID NO: 12) | (SEQ ID NO: 14) |
| 160F7_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 161E8_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 162C9_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 162D1_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 162G7_HC | TYAMN (SEQ ID NO: 2) | RIRSKYNNYATYYADSVKD | HGNFGNSYVSWFAY |
| | | (SEQ ID NO: 4) | (SEQ ID NO: 14) |
| 162G11_HC | TYAMN (SEQ ID NO: 2) | RIRSKYNNYATYYADSVKG | HGNFGNSYVSWFAY |
| | | (SEQ ID NO: 7) | (SEQ ID NO: 14) |
| 162H12_HC | TYAMN (SEQ ID NO: 2) | RIRSKYNNYATYYADPVKD | HGNFGNSYVSWFAY |
| | | (SEQ ID NO: 12) | (SEQ ID NO: 14) |

TABLE 2
Light Chain CDR Sequences

| Clone ID | CDRL1 | CDRL2 | CDRL3 |
|---|---|---|---|
| huLRRC15_Vkv1 | RASQDISNYLN (SEQ ID | YTSRLHS (SEQ ID NO: 19) | QQGEALPWT (SEQ ID |
| | NO: 15) (CDR1k) | (CDR2k) | NO: 22) (CDR3k) |
| 1B4_LC | GSSTGAVTTSNYAN | GTNKRAP (SEQ ID NO: 20) | ALWYSNLWV (SEQ ID |
| | (SEQ ID NO: 16) | | NO: 23) |
| 1B10_LC | GSSTGAVTTSNYAN | GTNKRAP (SEQ ID NO: 20) | ALWYSNLWV (SEQ ID |
| | (SEQ ID NO: 16) | | NO: 23) |
| 4A4_LC | RSSTGAVTTSNYAN | GTNKRAP (SEQ ID NO: 20) | ALWYSNLWV (SEQ ID |
| | (SEQ ID NO: 17) | | NO: 23) |
| 4A5_LC | GSSTGAVTTSNYAN | GTNKRAS (SEQ ID NO: 21) | ALWYSNLWV (SEQ ID |
| | (SEQ ID NO: 16) | | NO: 23) |
| 4A9_LC | RSSTGAVTTSNYAN | GTNKRAP (SEQ ID NO: 20) | ALWYSNLWV (SEQ ID |
| | (SEQ ID NO: 17) | | NO: 23) |
| 4A10_LC | RSSTGAVTTSNYAN | GTNKRAP (SEQ ID NO: 20) | ALWYSNLWV (SEQ ID |
| | (SEQ ID NO: 17) | | NO: 23) |
| 4A10.1_LC | RSSTGAVTTSNYAN | GTNKRAP (SEQ ID NO: 20) | ALWYSNLWV (SEQ ID |
| | (SEQ ID NO: 17) | | NO: 23) |
| 4A11_LC | GSSTGAVTTSNYAN | GTNKRAP (SEQ ID NO: 20) | ALWYSNLWV (SEQ ID |
| | (SEQ ID NO: 16) | | NO: 23) |
| 4A11.1_LC | GSSTGAVTTSNYAN | GTNKRAP (SEQ ID NO: 20) | ALWYSNLWV (SEQ ID |
| | (SEQ ID NO: 16) | | NO: 23) |
| 4A12_LC | GSSTGAVTTSNYAN | GTNKRAP (SEQ ID NO: 20) | ALWYSNLWV (SEQ ID |
| | (SEQ ID NO: 16) | | NO: 23) |
| 4A12.1_LC | GSSTGAVTTSNYAN | GTNKRAP (SEQ ID NO: 20) | ALWYSNLWV (SEQ ID |
| | (SEQ ID NO: 16) | | NO: 23) |
| 4B9_LC | GSSTGAVTTSNYAN | GTNKRAP (SEQ ID NO: 20) | ALWYSNLWV (SEQ ID |
| | (SEQ ID NO: 16) | | NO: 23) |
| 4B9.1_LC | GSSTGAVTTSNYAN | GTNKRAP (SEQ ID NO: 20) | ALWYSNLWV (SEQ ID |
| | (SEQ ID NO: 16) | | NO: 23) |
| 4B12_LC | RSSTGAVTTSNYAN | GTNKRAP (SEQ ID NO: 20) | ALWYSNLWV (SEQ ID |
| | (SEQ ID NO: 17) | | NO: 23) |
| 4B12.1_LC | RSSTGAVTTSNYAN | GTNKRAP (SEQ ID NO: 20) | ALWYSNLWV (SEQ ID |
| | (SEQ ID NO: 17) | | NO: 23) |
| 4C7_LC | GSSTGAVTTSNYAN | GTNKRAP (SEQ ID NO: 20) | ALWYSNLWV (SEQ ID |
| | (SEQ ID NO: 16) | | NO: 23) |
| 4C12_LC | GSSTGAVTNSNYAN | GTNKRAP (SEQ ID NO: 20) | ALWYSNLWV (SEQ ID |
| | (SEQ ID NO: 18) | | NO: 23) |
| 4D4_LC | GSSTGAVTTSNYAN | GTNKRAP (SEQ ID NO: 20) | ALWYSNLWV (SEQ ID |
| | (SEQ ID NO: 16) | | NO: 23) |
| 4D6_LC | GSSTGAVTTSNYAN | GTNKRAP (SEQ ID NO: 20) | ALWYSNLWV (SEQ ID |
| | (SEQ ID NO: 16) | | NO: 23) |
| 4D6.1_LC | GSSTGAVTTSNYAN | GTNKRAP (SEQ ID NO: 20) | ALWYSNLWV (SEQ ID |
| | (SEQ ID NO: 16) | | NO: 23) |
| 4D7_LC | GSSTGAVTTSNYAN | GTNKRAP (SEQ ID NO: 20) | ALWYSNLWV (SEQ ID |
| | (SEQ ID NO: 16) | | NO: 23) |
| 4D9_LC | GSSTGAVTTSNYAN | GTNKRAP (SEQ ID NO: 20) | ALWYSNLWV (SEQ ID |
| | (SEQ ID NO: 16) | | NO: 23) |
| 4E7_LC | GSSTGAVTTSNYAN | GTNKRAP (SEQ ID NO: 20) | ALWYSNLWV (SEQ ID |
| | (SEQ ID NO: 16) | | NO: 23) |
| 4F5_LC | GSSTGAVTTSNYAN | GTNKRAP (SEQ ID NO: 20) | ALWYSNLWV (SEQ ID |
| | (SEQ ID NO: 16) | | NO: 23) |
| 4F11_LC | GSSTGAVTTSNYAN | GTNKRAP (SEQ ID NO: 20) | ALWYSNLWV (SEQ ID |
| | (SEQ ID NO: 16) | | NO: 23) |
| 4G1_LC | GSSTGAVTTSNYAN | GTNKRAP (SEQ ID NO: 20) | ALWYSNLWV (SEQ ID |
| | (SEQ ID NO: 16) | | NO: 23) |
| 4G2_LC | GSSTGAVTTSNYAN | GTNKRAP (SEQ ID NO: 20) | ALWYSNLWV (SEQ ID |
| | (SEQ ID NO: 16) | | NO: 23) |
| 160C9_LC | RSSTGAVTTSNYAN | GTNKRAP (SEQ ID NO: 20) | ALWYSNLWV (SEQ ID |
| | (SEQ ID NO: 17) | | NO: 23) |
| 160D10_LC | GSSTGAVTTSNYAN | GTNKRAP (SEQ ID NO: 20) | ALWYSNLWV (SEQ ID |
| | (SEQ ID NO: 16) | | NO: 23) |
| 160F1_LC | RSSTGAVTTSNYAN | GTNKRAP (SEQ ID NO: 20) | ALWYSNLWV (SEQ ID |
| | (SEQ ID NO: 17) | | NO: 23) |
| 160H4_LC | GSSTGAVTTSNYAN | GTNKRAP (SEQ ID NO: 20) | ALWYSNLWV (SEQ ID |
| | (SEQ ID NO: 16) | | NO: 23) |
| 161A8_LC | GSSTGAVTTSNYAN | GTNKRAP (SEQ ID NO: 20) | ALWYSNLWV (SEQ ID |
| | (SEQ ID NO: 16) | | NO: 23) |
| 161B4_LC | GSSTGAVTTSNYAN | GTNKRAP (SEQ ID NO: 20) | ALWYSNLWV (SEQ ID |
| | (SEQ ID NO: 16) | | NO: 23) |
| 161E6_LC | RSSTGAVTTSNYAN | GTNKRAP (SEQ ID NO: 20) | ALWYSNLWV (SEQ ID |
| | (SEQ ID NO: 17) | | NO: 23) |
| 161F8_LC | RSSTGAVTTSNYAN | GTNKRAP (SEQ ID NO: 20) | ALWYSNLWV (SEQ ID |
| | (SEQ ID NO: 17) | | NO: 23) |
| 161G1_LC | RSSTGAVTTSNYAN | GTNKRAP (SEQ ID NO: 20) | ALWYSNLWV (SEQ ID |
| | (SEQ ID NO: 17) | | NO: 23) |
| 161H2_LC | GSSTGAVTTSNYAN | GTNKRAP (SEQ ID NO: 20) | ALWYSNLWV (SEQ ID |
| | (SEQ ID NO: 16) | | NO: 23) |
| 162B10_LC | GSSTGAVTTSNYAN | GTNKRAP (SEQ ID NO: 20) | ALWYSNLWV (SEQ ID |
| | (SEQ ID NO: 16) | | NO: 23) |
| 162B12_LC | RSSTGAVTTSNYAN | GTNKRAP (SEQ ID NO: 20) | ALWYSNLWV (SEQ ID |
| | (SEQ ID NO: 17) | | NO: 23) |
| 162D3_LC | GSSTGAVTTSNYAN | GTNKRAP (SEQ ID NO: 20) | ALWYSNLWV (SEQ ID |
| | (SEQ ID NO: 16) | | NO: 23) |
| 162D11_LC | GSSTGAVTTSNYAN | GTNKRAP (SEQ ID NO: 20) | ALWYSNLWV (SEQ ID |
| | (SEQ ID NO: 16) | | NO: 23) |
| 162G12_LC | GSSTGAVTTSNYAN | GTNKRAP (SEQ ID NO: 20) | ALWYSNLWV (SEQ ID |
| | (SEQ ID NO: 16) | | NO: 23) |
| 160D12_LC | GSSTGAVTTSNYAN | GTNKRAP (SEQ ID NO: 20) | ALWYSNLWV (SEQ ID |
| | (SEQ ID NO: 16) | | NO: 23) |
| 160E2_LC | GSSTGAVTTSNYAN | GTNKRAP (SEQ ID NO: 20) | ALWYSNLWV (SEQ ID |
| | (SEQ ID NO: 16) | | NO: 23) |
| 160E12_LC | GSSTGAVTTSNYAN | GTNKRAP (SEQ ID NO: 20) | ALWYSNLWV (SEQ ID |
| | (SEQ ID NO: 16) | | NO: 23) |
| 160F2_LC | GSSTGAVTTSNYAN | GTNKRAP (SEQ ID NO: 20) | ALWYSNLWV (SEQ ID |
| | (SEQ ID NO: 16) | | NO: 23) |
| 160F7_LC | GSSTGAVTTSNYAN | GTNKRAP (SEQ ID NO: 20) | ALWYSNLWV (SEQ ID |
| | (SEQ ID NO: 16) | | NO: 23) |
| 161E8_LC | GSSTGAVTTSNYAN | GTNKRAP (SEQ ID NO: 20) | ALWYSNLWV (SEQ ID |
| | (SEQ ID NO: 16) | | NO: 23) |
| 162C9_LC | GSSTGAVTTSNYAN | GTNKRAP (SEQ ID NO: 20) | ALWYSNLWV (SEQ ID |
| | (SEQ ID NO: 16) | | NO: 23) |
| 162D1_LC | RSSTGAVTTSNYAN | GTNKRAP (SEQ ID NO: 20) | ALWYSNLWV (SEQ ID |
| | (SEQ ID NO: 17) | | NO: 23) |
| 162G7_LC | GSSTGAVTTSNYAN | GTNKRAP (SEQ ID NO: 20) | ALWYSNLWV (SEQ ID |
| | (SEQ ID NO: 16) | | NO: 23) |
| 162G11_LC | GSSTGAVTTSNYAN | GTNKRAP (SEQ ID NO: 20) | ALWYSNLWV (SEQ ID |
| | (SEQ ID NO: 16) | | NO: 23) |
| 162H12_LC | GSSTGAVTTSNYAN | GTNKRAP (SEQ ID NO: 20) | ALWYSNLWV (SEQ ID |
| | (SEQ ID NO: 16) | | NO: 23) |

TABLE 3
Anti-LRRC15 heavy chain variable region sequences

| Clone ID | VH sequence |
|---|---|
| huLRRC15_V | QVQLVQSGAEVKKPGASVKVSCKASGYTFTSYWIEWV |
| Hv1 | RQAPGQGLEWMGEILPGSDTTNYNEKFKDRVTMTRDT |
| | STSTVYMELSSLRSEDTAVYYCARDRGNYRAWFGYWG |
| | QGTLVTVSS |
| | (SEQ ID NO: 24) |
| huLRRC15_V | QVQLVQSGAEVKKPGASVKVSCKASGYKFTSYWIEWV |
| Hv2 | RQAPGQGLEWIGEILPGSDTTNYNEKFKDRVTFTSDT |
| | STSTVYMELSSLRSEDTAVYYCARDRGNYRAWFGYWG |
| | QGTLVTVSS |
| | (SEQ ID NO: 25) |
| huLRRC15_V | QVQLVQSGAEVKKPGASVKVSCKASGYKFSSYWIEWV |
| Hv3 | RQAPGQGLEWIGEILPGSDTTNYNEKFKDKATFTSDT |
| | STSTAYMELSSLRSEDTAVYYCARDRGNYRAWFGYWG |
| | QGTLVTVSS |
| | (SEQ ID NO: 26) |

TABLE 4
Anti-LRRC15 light chain variable region sequences

| Clone ID | VL sequence |
|---|---|
| huLRRC15_V | DIQMTQSPSSLSASVGDRVTITCRASQDISNYLNWYQQ |
| Kv1 | KPGKAPKFLIYYTSRLHSGVPSRFSGSGSGTDYTLTIS |
| | SLQPEDFATYYCQQGEALPWTFGQGTKVEIK |
| | (SEQ ID NO: 27) |

TABLE 5
Anti-CD3 VH sequences

| Clone ID | Sequence |
|---|---|
| 1B4_HC | EVKLVESGGGLVQPGGSLKLSCAASGFTFNTYAMNWVRQAPGKGLEWVGRIRSKYNNYAT |
| | YYADSVKDRFTISRDDSQNILYLQMNNLRTEDTAVYYCVRHGNFGNSYVSWFAYWGQGTL |
| | VTVSS (SEQ ID NO: 28) |
| 1B10_HC | EVKLLESGGGLVQPGGSLKLSCAASGFTFNTYAMNWVRQAPGKGLEWVGRIRSKANNYAT |
| | YYADSVKGRFTISRDDSKNTLYLQMNNLKAEDTAVYYCVRHGNFGNSYVSWFAYWGQGTL |
| | VTVSS (SEQ ID NO: 29) |
| 4A4_HC | EVKLVESGGGLVQPGGSLKLSCAASGFTFNTYAMNWVRQAPGKGLEWVGRIRSKYNNYAT |
| | YYADPVKGRFTISRDDAQNTLYLQMNNLKAEDTAVYYCVRHGNFGNSYVSWFAYWGQGTL |
| | VTVSS (SEQ ID NO: 30) |
| 4A5_HC | EVQLVESGGGLVQPGGSLKLSCAASGFTFNTYAMNWVRQAPGKGLEWVGRIRSKYNNYAT |
| | YYADSVKDRFTISRDDAKNTLYLQMNNLRAEDTAVYYCVRHGNFGNSYVSWFAYWGQGTL |
| | VTVSS (SEQ ID NO: 31) |
| 4A9_HC | EVKLVESGGGLVQPGGSLKLSCAASGFTFNTYAMNWVRQAPGKGLEWVGRIRSKYNNYAT |
| | YYADSVKGRFTISRDDSKNILYLQMNNLKAEDTAVYYCVRHGNFGNSYVSWFAYWGQGTL |
| | VTVSS (SEQ ID NO: 32) |
| 4A10_HC | EVKLVESGGGLVQPGGSLKLSCAASGFTFNTYAMNWVRQAPGKGLEWVGRIRSKYNNYAT |
| | YYADSVKDRFTISRDDSKNTLYLQMNNLRAEDTAVYYCVRHGNFGNSYVSWFAYWGQGTL |
| | VTVSS (SEQ ID NO: 33) |
| 4A10.1_HC | EVKLVESGGGLVQPGGSLKLSCAASGFTFNTYAMNWVRQAPGKGLEWVGRIRSKYNNYAT |
| | YYADSVKDRFTISRDDSKNTLYLQMNNLRAEDTAVYYCVRHGNFGNSYVSWFAYWGQGTL |
| | VTVSS (SEQ ID NO: 34) |
| 4A11_HC | EVKLVESGGGLVQPGGSLKLSCAASGFTFNTYAMNWVRQAPGKGLEWVGRIRSKYNNYAT |
| | YYAASVKDRFTISRDDSKNTLYLQMNNLRAEDTAVYYCVRHGNFGNSYVSWFAYWGQGTL |
| | VTVSS (SEQ ID NO: 35) |
| 4A11.1_HC | EVKLVESGGGLVQPGGSLKLSCAASGFTFNTYAMNWVRQAPGKGLEWVGRIRSKYNNYAT |
| | YYADSVKDRFTISRDDSKNTLYLQMNNLRAEDTAVYYCVRHGNFGNSYVSWFAYWGQGTL |
| | VTVSS (SEQ ID NO: 36) |
| 4A12_HC | EVQLVESGGGLVQPGGSLKLSCAASGFTFNTYAMNWVRQAPGKGLEWVGRIRSKYNNYAT |
| | YYADSVKDRFTISRDDAKNTLYLQMNNLKAEDTAVYYCVRHGNFGNSYVSWFAYWGQGTL |
| | VTVSS (SEQ ID NO: 37) |
| 4A12.1_HC | EVQLVESGGGLVQPGGSLKLSCAASGFTFNTYAMNWVRQAPGKGLEWVGRIRSKYNNYAT |
| | YYADSVKDRFTISRDDAKNTLYLQMNNLKAEDTAVYYCVRHGNFGNSYVSWFAYWGQGTL |
| | VTVSS (SEQ ID NO: 38) |
| 4B9_HC | EVQLLESGGGLVQPGGSLKLSCAASGFTFNTYAMNWVRQAPGKGLEWVGRIRSKANNYAT |
| | YYADSVKGRFTISRDDSKNILYLQMNSLRAEDTAVYYCVRHGNFGNSYVSWFAYWGQGTL |
| | VTVSS (SEQ ID NO: 39) |
| 4B9.1_HC | EVQLLESGGGLVQPGGSLKLSCAASGFTFNTYAMNWVRQAPGKGLEWVGRIRSKANNYAT |
| | YYADSVKGRFTISRDDSKNILYLQMNSLRAEDTAVYYCVRHGNFGNSYVSWFAYWGQGTL |
| | VTVSS (SEQ ID NO: 40) |
| 4B12_HC | EVKLVESGGGLVQPGGSLKLSCAASGFTFNTYAMNWVRQAPGKGLEWVGRIRSKYNNYAT |
| | YYADSVKDRFTISRDDSKNTLYLQMNNLKAEDTAVYYCVRHGNFGNSYVSWFAYWGQGTL |
| | VTVSS (SEQ ID NO: 41) |
| 4B12.1_HC | EVKLVESGGGLVQPGGSLKLSCAASGFTFNTYAMNWVRQAPGKGLEWVGRIRSKYNNYAT |
| | YYADSVKDRFTISRDDSKNTLYLQMNNLKAEDTAVYYCVRHGNFGNSYVSWFAYWGQGTL |
| | VTVSS (SEQ ID NO: 42) |
| 4C7_HC | EVRLVESGGGLVQPGGSLKLSCAASGFTFNTYAMNWVRQAPGKGLEWVGRIRSKINNYAT |
| | YYADSVKDRFTISRDDAQNILYLQMNNLRAEDTAVYYCVRHGNFGNSYVSWFAYWGQGTL |
| | VTVSS (SEQ ID NO: 43) |
| 4C12_HC | EVKLVESGGGLVQPGGSLKLSCAASGFTFNTYAMNWVRQAPGKGLEWVGRIRSKSNNYAT |
| | YYAASVKDRFTISRDDSKNSLYLQMNNLKAEDTAVYYCVRHGNFGNSYVSWFAYWGQGTL |
| | VTVSS (SEQ ID NO: 44) |
| 4D4_HC | EVKLVESGGGLVQPGGSLKLSCAASGFTFNTYAMNWVRQAPGKGLEWVGRIRSKYNNYAT |
| | YYADSVKGRFTISRDDSKNTLYLQMNNLKTEDTAVYYCVRHGNFGNSYVSWFAYWGQGTL |
| | VTVSS (SEQ ID NO: 45) |
| 4D6_HC | EVKLVESGGGLVQPGGSLKLSCAASGFTFNTYAMNWVRQAPGKGLEWVGRIRSKSNNYAT |
| | YYADSVKDRFTISRDDSKNILYLQMNNLKAEDTAVYYCVRHGNFGNSYVSWFAYWGQGTL |
| | VTVSS (SEQ ID NO: 46) |
| 4D6.1_HC | EVKLVESGGGLVQPGGSLKLSCAASGFTFNTYAMNWVRQAPGKGLEWVGRIRSKSNNYAT |
| | YYADSVKDRFTISRDDSKNILYLQMNNLKAEDTAVYYCVRHGNFGNSYVSWFAYWGQGTL |
| | VTVSS (SEQ ID NO: 47) |
| 4D7_HC | EVKLVESGGGLVQPGGSLKLSCAASGFTFNTYAMNWVRQAPGKGLEWVGRIRSKYNNYAT |
| | YYADSVKDRFTISRDDAQNILYLQMNNLKTEDTAVYYCVRHGNFGNSYVSWFAYWGQGTL |
| | VTVSS (SEQ ID NO: 48) |
| 4D9_HC | EVKLVESGGGLVQPGGSLKLSCAASGFTFNTYAMNWVRQAPGKGLEWVGRIRSKYNNYAT |
| | YYADSVKGRFTISRDDSQSILYLQMNNLRAEDTAVYYCVRHGNFGNSYVSWFAYWGQGTL |
| | VTVSS (SEQ ID NO: 49) |
| 4E7_HC | EVKLVESGGGLVQPGGSLKLSCAASGFTFNTYAMNWVRQAPGKGLEWVGRIRSKYNNYAT |
| | YYADSVKDRFTISRDDSQNILYLQMNNLKAEDTAVYYCVRHGNFGNSYVSWFAYWGQGTL |
| | VTVSS (SEQ ID NO: 50) |
| 4F5_HC | EVKLVESGGGLVQPGGSLKLSCAASGFTFNTYAMNWVRQAPGKGLEWVGRIRSKYNNYAT |
| | YYADPVKGRFTISRDDAKNTLYLQMNSLKAEDTAVYYCVRHGNFGNSYVSWFAYWGQGTL |
| | VTVSS (SEQ ID NO: 51) |
| 4F11_HC | EVKLVESGGGLVQPGGSLKLSCAASGFTFNTYAMNWVRQAPGKGLEWVGRIRSKYNNYAT |
| | YYADSVKDRFTISRDDSKSTLYLQMNNLRTEDTAVYYCVRHGNFGNSYVSWFAYWGQGTL |
| | VTVSS (SEQ ID NO: 52) |
| 4G1_HC | EVKLVESGGGLVQPGGSLKLSCAASGFTFNTYAMNWVRQAPGKGLEWVGRIRSKYNNYAT |
| | YYADSVKGRFTISRDDAQNILYLQMNNLKTEDTAVYYCVRHGNFGNSYVSWFAYWGQGTL |
| | VTVSS (SEQ ID NO: 53) |
| 4G2_HC | EVKLLESGGGLVQPGGSLKLSCAASGFTFNTYAMNWVRQAPGKGLEWVGRIRSKYNNYAT |
| | YYADSVKGRFTISRDDAKSILYLQMNNLKTEDTAVYYCVRHGNFGNSYVSWFAYWGQGTL |
| | VTVSS (SEQ ID NO: 54) |
| 160C9_HC | EVKLVESGGGLVQPGGSLKLSCAASGFTFNTYAMNWVRQAPGKGLEWVARIRSKYNNYAT |
| | YYADSVKDRFTISRDDAKNTLYLQMNNLRTEDTAVYYCVRHGNFGNSYVSWFAYWGQGTL |
| | VTVSS (SEQ ID NO: 55) |
| 160D10_HC | EVQLVESGGGLVQPGGSLKLSCAASGFTFNTYAMNWVRQAPGKGLEWVGRIRSKYNNYAT |
| | YYADSVKGRFTISRDDAKSTLYLQMNSLRTEDTAVYYCVRHGNFGNSYVSWFAYWGQGTL |
| | VTVSS (SEQ ID NO: 56) |
| 160F1_HC | EVQLLESGGGLVQPGGSLKLSCAASGFTFNTYAMNWVRQAPGKGLEWVARIRSKYNNYAT |
| | YYADSVKDRFTISRDDSQNILYLQMNNLKTEDTAVYYCVRHGNFGNSYVSWFAYWGQGTL |
| | VTVSS (SEQ ID NO: 57) |
| 160H4_HC | EVKLVESGGGLVQPGGSLKLSCAASGFTFNTYAMNWVRQAPGKGLEWVARIRSKYNNYAT |
| | YYADSVKDRFTISRDDSKNTLYLQMNSLKTEDTAVYYCVRHGNFGNSYVSWFAYWGQGTL |
| | VTVSS (SEQ ID NO: 58) |
| 161A8_HC | EVQLVESGGGLVQPGGSLKLSCAASGFTFNTYAMNWVRQAPGKGLEWVARIRSKYNNYAT |
| | YYADSVKDRFTISRDDSKNILYLQMNSLRTEDTAVYYCVRHGNFGNSYVSWFAYWGQGTL |
| | VTVSS (SEQ ID NO: 59) |
| 161B4_HC | EVQLVESGGGLVQPGGSLKLSCAASGFTFNTYAMNWVRQAPGKGLEWVGRIRSKYNNYAT |
| | YYADSVKDRFTISRDDAQNILYLQMNNLKTEDTAVYYCVRHGNFGNSYVSWFAYWGQGTL |
| | VTVSS (SEQ ID NO: 60) |
| 161E6_HC | EVQLVESGGGLVQPGGSLKLSCAASGFTFNTYAMNWVRQAPGKGLEWVGRIRSKYNNYAT |
| | YYADSVKGRFTISRDDSKSSLYLQMNSLKAEDTAVYYCVRHGNFGNSYVSWFAYWGQGTL |
| | VTVSS (SEQ ID NO: 61) |
| 161F8_HC | EVKLVESGGGLVQPGGSLKLSCAASGFTFNTYAMNWVRQAPGKGLEWVGRIRSKYNNYAT |
| | YYADSVKGRFTISRDDSKNILYLQMNNLKAEDTAVYYCVRHGNFGNSYVSWFAYWGQGTL |
| | VTVSS (SEQ ID NO: 62) |
| 161G1_HC | EVKLVESGGGLVQPGGSLKLSCAASGFTFNTYAMNWVRQAPGKGLEWVGRIRSKYNNYAT |
| | YYADSVKGRFTISRDDSQNILYLQMNNLRTEDTAVYYCVRHGNFGNSYVSWFAYWGQGTL |
| | VTVSS (SEQ ID NO: 63) |
| 161H2_HC | EVKLVESGGGLVQPGGSLKLSCAASGFTFNTYAMNWVRQAPGKGLEWVARIRSKYNNYAT |
| | YYADSVKDRFTISRDDAKNILYLQMNNLRTEDTAVYYCVRHGNFGNSYVSWFAYWGQGTL |
| | VTVSS (SEQ ID NO: 64) |
| 162B10_HC | EVQLVESGGGLVQPGGSLKLSCAASGFTFNTYAMNWVRQAPGKGLEWVGRIRSKYNNYAT |
| | YYADSVKDRFTISRDDAKSILYLQMNNLRAEDTAVYYCVRHGNFGNSYVSWFAYWGQGTL |
| | VTVSS (SEQ ID NO: 65) |
| 162B12_HC | EVKLVESGGGLVQPGGSLKLSCAASGFTFNTYAMNWVRQAPGKGLEWVGRIRSKYNNYAT |
| | YYADSVKDRFTISRDDSKNILYLQMNNLRTEDTAVYYCVRHGNFGNSYVSWFAYWGQGTL |
| | VTVSS (SEQ ID NO: 66) |
| 162D3_HC | EVQLLESGGGLVQPGGSLKLSCAASGFTFNTYAMNWVRQAPGKGLEWVGRIRSKYNNYAT |
| | YYADSVKGRFTISRDDSQNTLYLQMNNLKTEDTAVYYCVRHGNFGNSYVSWFAYWGQGTL |
| | VTVSS (SEQ ID NO: 67) |
| 162D11_HC | EVKLVESGGGLVQPGGSLKLSCAASGFTFNTYAMNWVRQAPGKGLEWVGRIRSKYNNYAT |
| | YYADPVKGRFTISRDDSKSTLYLQMNNLRTEDTAVYYCVRHGNFGNSYVSWFAYWGQGTL |
| | VTVSS (SEQ ID NO: 68) |
| 162G12_HC | EVQLVESGGGLVQPGGSLKLSCAASGFTFNTYAMNWVRQAPGKGLEWVGRIRSKYNNYAT |
| | YYADSVKDRFTISRDDAKSTLYLQMNNLRAEDTAVYYCVRHGNFGNSYVSWFAYWGQGTL |
| | VTVSS (SEQ ID NO: 69) |
| 160D12_HC | EVQLVESGGGLVQPGGSLKLSCAASGFTFNTYAMNWVRQAPGKGLEWVGRIRSKYNNYAT |
| | YYADSVKGRFTISRDDSQSTLYLQMNSLRAEDTAVYYCVRHGNFGNSYVSWFAYWGQGTL |
| | VTVSS (SEQ ID NO: 70) |
| 160E2_HC | EVQLVESGGGLVQPGGSLKLSCAASGFTFNTYAMNWVRQAPGKGLEWVARIRSKYNNYAT |
| | YYADSVKGRFTISRDDSKNTLYLQMNNLKAEDTAVYYCVRHGNFGNSYVSWFAYWGQGTL |
| | VTVSS (SEQ ID NO: 71) |
| 160E12_HC | EVQLLESGGGLVQPGGSLKLSCAASGFTFNTYAMNWVRQAPGKGLEWVGRIRSKYNNYAT |
| | YYADSVKDRFTISRDDSKSTLYLQMNNLKAEDTAVYYCVRHGNFGNSYVSWFAYWGQGTL |
| | VTVSS (SEQ ID NO: 72) |
| 160F2_HC | EVKLVESGGGLVQPGGSLKLSCAASGFTFNTYAMNWVRQAPGKGLEWVSRIRSKYNNYAT |
| | YYADPVKDRFTISRDDAQNTLYLQMNNLKAEDTAVYYCVRHGNFGNSYVSWFAYWGQGTL |
| | VTVSS (SEQ ID NO: 73) |
| 160F7_HC | EVKLVESGGGLVQPGGSLKLSCAASGFTFNTYAMNWVRQAPGKGLEWVARIRSKYNNYAT |
| | YYADSVKGRFTISRDDSKNILYLQMNSLKAEDTAVYYCVRHGNFGNSYVSWFAYWGQGTL |
| | VTVSS (SEQ ID NO: 74) |
| 161E8_HC | EVKLVESGGGLVQPGGSLKLSCAASGFTFNTYAMNWVRQAPGKGLEWVGRIRSKYNNYAT |
| | YYADSVKGRFTISRDDAKSILYLQMNNLKAEDTAVYYCVRHGNFGNSYVSWFAYWGQGTL |
| | VTVSS (SEQ ID NO: 75) |
| 162C9_HC | EVKLVESGGGLVQPGGSLKLSCAASGFTFNTYAMNWVRQAPGKGLEWVGRIRSKYNNYAT |
| | YYADSVKDRFTISRDDSQNTLYLQMNNLKAEDTAVYYCVRHGNFGNSYVSWFAYWGQGTL |
| | VTVSS (SEQ ID NO: 76) |
| 162D1_HC | EVQLVESGGGLVQPGGSLKLSCAASGFTFNTYAMNWVRQAPGKGLEWVGRIRSKYNNYAT |
| | YYADSVKGRFTISRDDAKSTLYLQMNNLKTEDTAVYYCVRHGNFGNSYVSWFAYWGQGTL |
| | VTVSS (SEQ ID NO: 77) |
| 162G7_HC | EVKLVESGGGLVQPGGSLKLSCAASGFTFNTYAMNWVRQAPGKGLEWVARIRSKYNNYAT |
| | YYADSVKGRFTISRDDAKNSLYLQMNNLKAEDTAVYYCVRHGNFGNSYVSWFAYWGQGTL |
| | VTVSS (SEQ ID NO: 78) |
| 162G11_HC | EVKLVESGGGLVQPGGSLKLSCAASGFTFNTYAMNWVRQAPGKGLEWVGRIRSKYNNYAT |
| | YYADSVKGRFTISRDDAKNSLYLQMNNLKAEDTAVYYCVRHGNFGNSYVSWFAYWGQGTL |
| | VTVSS (SEQ ID NO: 79) |
| 162H12_HC | EVKLVESGGGLVQPGGSLKLSCAASGFTFNTYAMNWVRQAPGKGLEWVGRIRSKYNNYAT |
| | YYADPVKDRFTISRDDSQNILYLQMNNLKAEDTAVYYCVRHGNFGNSYVSWFAYWGQGTL |
| | VTVSS (SEQ ID NO: 80) |

TABLE 6
Anti-CD3 VL sequences

| Clone ID | Sequence |
|---|---|
| 1B4_LC | QAFVTQEPSLTVSPGGTVTLTCGSSTGAVTTSNYANWVQQKPGQAPRGLIGGTNKRAPGVPA |
| | RFSGSLIGDKAALTITGAQPEDEAEYYCALWYSNLWVFGGGTKLTVL (SEQ ID NO: 81) |
| 1B10_LC | EAVVTQEPSLTVSPGGTVTLTCGSSTGAVTTSNYANWVQQKPGQAPRGLIGGTNKRAPGTPA |
| | RFSGSLLGDKAALTITGAQPEDEAEYYCALWYSNLWVFGGGTKLTVL (SEQ ID NO: 82) |
| 4A4_LC | EAVVTQEPSLTVSPGGTVTLTCRSSTGAVTTSNYANWVQQKPGQAFRGLIGGTNKRAPGVPA |
| | RFSGSLLGDKAALTIAGAQPEDEAEYYCALWYSNLWVFGGGTKLTVL (SEQ ID NO: 83) |
| 4A5_LC | QAVVTQEPSLTVSPGGTVTLTCGSSTGAVTTSNYANWVQQKPGQAFRGLIGGTNKRASGVPA |
| | RFSGSLIGDKAALTIAGAQPEDEAEYYCALWYSNLWVFGGGTKLTVL (SEQ ID NO: 84) |
| 4A9_LC | QAVVTQEPSLTVSPGGTVTLTCRSSTGAVTTSNYANWVQQKPGQAPRGLIGGTNKRAPGTPA |
| | RFSGSLIGDKAALTITGVQPEDEAEYYCALWYSNLWVFGGGTKLTVL (SEQ ID NO: XX) |
| 4A10_LC | EAVVTQEPSLTVSPGGTVTLTCRSSTGAVTTSNYANWVQQKPGQAFRGLIGGTNKRAPGVPA |
| | RFSGSLIGDKAALTITGAQPEDEAEYYCALWYSNLWVFGGGTKLTVL (SEQ ID NO: 85) |
| 4A10.1_LC | EAVVTQEPSLTVSPGGTVTLTCRSSTGAVTTSNYANWVQQKPGQAPRGLIGGTNKRAPGVPA |
| | RFSGSLLGGKAALTLSGAQPEDEAEYYCALWYSNLWVFGGGTKLTVL (SEQ ID NO: 86) |
| 4A11_LC | QAVVTQEPSLTVSPGGTVTLTCGSSTGAVTTSNYANWVQQKPGQAFRGLIGGTNKRAPGVPA |
| | RFSGSLLGGKAALTITGAQPEDEAEYYCALWYSNLWVFGGGTKLTVL (SEQ ID NO: 87) |
| 4A11.1_LC | QAVVTQEPSLTVSPGGTVTLTCGSSTGAVTTSNYANWVQQKPGQAPRGLIGGTNKRAPGVPA |
| | RFSGSLLGGKAALTLSGAQPEDEAEYYCALWYSNLWVFGGGTKLTVL (SEQ ID NO: 88) |
| 4A12_LC | QAVVTQEPSLTVSPGGTVTLTCGSSTGAVTTSNYANWVQQKPGQAFRGLIGGTNKRAPGVPA |
| | RFSGSLLGDKAALTITGAQPEDEAEYYCALWYSNLWVFGGGTKLTVL (SEQ ID NO: 89) |
| 4A12.1_LC | QAVVTQEPSLTVSPGGTVTLTCGSSTGAVTTSNYANWVQQKPGQAPRGLIGGTNKRAPGVPA |
| | RFSGSLLGGKAALTLSGAQPEDEAEYYCALWYSNLWVFGGGTKLTVL (SEQ ID NO: 90) |
| 4B9_LC | QAVVTQEPSLTVSPGGTVTLTCGSSTGAVTTSNYANWVQQKPGQAFRGLIGGTNKRAPGVPA |
| | RFSGSLIGGKAALTIAGAQPEDEAEYYCALWYSNLWVFGGGTKLTVL (SEQ ID NO: 91) |
| 4B9.1_LC | QAVVTQEPSLTVSPGGTVTLTCGSSTGAVTTSNYANWVQQKPGQAPRGLIGGTNKRAPGVPA |
| | RFSGSLLGGKAALTLAGAQPEDEAEYYCALWYSNLWVFGGGTKLTVL (SEQ ID NO: 92) |
| 4B12_LC | EAVVTQEPSLTVSPGGTVTLTCRSSTGAVTTSNYANWVQQKPGQAFRGLIGGTNKRAPGVPA |
| | RFSGSLIGDKAALTITGAQPEDEAEYYCALWYSNLWVFGGGTKLTVL (SEQ ID NO: 93) |
| 4B12.1_LC | EAVVTQEPSLTVSPGGTVTLTCRSSTGAVTTSNYANWVQQKPGQAPRGLIGGTNKRAPGVPA |
| | RFSGSLLGGKAALTLSGAQPEDEAEYYCALWYSNLWVFGGGTKLTVL (SEQ ID NO: 94) |
| 4C7_LC | QAVVTQEPSLTVSPGGTVTLTCGSSTGAVTTSNYANWVQQKPGQAPRGLIGGTNKRAPGVPA |
| | RFSGSLIGDKAALTIAGAQPEDEAEYYCALWYSNLWVFGGGTKLTVL (SEQ ID NO: 95) |
| 4C12_LC | QAVVTQEPSLTVSPGGTVTLTCGSSTGAVTNSNYANWVQQKPGQAFRGLIGGTNKRAPGTPA |
| | RFSGSLLGGKAALTIAGVQPEDEAEYYCALWYSNLWVFGGGTKLTVL (SEQ ID NO: 96) |
| 4D4_LC | QAVVTQEPSLTVSPGGTVTLTCGSSTGAVTTSNYANWVQQKPGQAPRGLIGGTNKRAPWTPA |
| | RFSGSLIGDKAALTIAGAQPEDEAEYYCALWYSNLWVFGGGTKLTVL (SEQ ID NO: 97) |
| 4D6_LC | EAVVTQEPSLTVSPGGTVTLTCGSSTGAVTTSNYANWVQQKPGQAFRGLIGGTNKRAPGTPA |
| | RFSGSLIGGKAALTIAGAQPEDEAEYYCALWYSNLWVFGGGTKLTVL (SEQ ID NO: 98) |
| 4D6.1_LC | EAVVTQEPSLTVSPGGTVTLTCGSSTGAVTTSNYANWVQQKPGQAPRGLIGGTNKRAPGTPA |
| | RFSGSLLGGKAALTLAGAQPEDEAEYYCALWYSNLWVFGGGTKLTVL (SEQ ID NO: 99) |
| 4D7_LC | EAVVTQEPSLTVSPGGTVTLTCGSSTGAVTTSNYANWVQQKPGQAFRGLIGGTNKRAPGVPA |
| | RFSGSLIGDKAALTLAGAQPEDEAEYYCALWYSNLWVFGGGTKLTVL (SEQ ID NO: 100) |
| 4D9_LC | EAVVTQEPSLTVSPGGTVTLTCGSSTGAVTTSNYANWVQQKPGQAFRGLIGGTNKRAPGVPA |
| | RFSGSLIGDKAALTITGAQPEDEAEYYCALWYSNLWVFGGGTKLTVL (SEQ ID NO: 101) |
| 4E7_LC | QAVVTQEPSLTVSPGGTVTLTCGSSTGAVTTSNYANWVQQKPGQAPRGLIGGTNKRAPGVPA |
| | RFSGSLIGGKAALTITGVQPEDEAEYYCALWYSNLWVFGGGTKLTVL (SEQ ID NO: 102) |
| 4F5_LC | QAVVTQEPSLTVSPGGTVTLTCGSSTGAVTTSNYANWVQQKPGQAFRGLIGGTNKRAPGVPA |
| | RFSGSLIGDKAALTITGAQPEDEAEYYCALWYSNLWVFGGGTKLTVL (SEQ ID NO: 103) |
| 4F11_LC | QAVVTQEPSLTVSPGGTVTLTCGSSTGAVTTSNYANWVQQKPGQAFRGLIGGTNKRAPGTPA |
| | RFSGSLIGDKAALTITGAQPEDEAEYYCALWYSNLWVFGGGTKLTVL (SEQ ID NO: 104) |
| 4G1_LC | QAVVTQEPSLTVSPGGTVTLTCGSSTGAVTTSNYANWVQQKPGQAFRGLIGGTNKRAPGTPA |
| | RFSGSLLGDKAALTITGAQPEDEAEYYCALWYSNLWVFGGGTKLTVL (SEQ ID NO: 105) |
| 4G2_LC | EAVVTQEPSLTVSPGGTVTLTCGSSTGAVTTSNYANWVQQKPGQAFRGLIGGTNKRAPGTPA |
| | RFSGSLIGGKAALTITGAQPEDEAEYYCALWYSNLWVFGGGTKLTVL (SEQ ID NO: 106) |
| 160C9_LC | QAVVTQEPSLTVSPGGTVTLTCRSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 107) |
| 160D10_LC | QAVVTQEPSLTVSPGGTVTLTCGSSTGAVTTSNYANWVQQKPGQAFRGLIGGTNKRAPGVPA |
| | RFSGSLLGGKAALTLSGAQPEDEAEYYCALWYSNLWVFGGGTKLTVL (SEQ ID NO: 108) |
| 160F1_LC | QAVVTQEPSLTVSPGGTVTLTCRSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 109) |
| 160H4_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 110) |
| 161A8_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 111) |
| 161B4_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 112) |
| 161E6_LC | QAVVTQEPSLTVSPGGTVTLTCRSSTGAVTTSNYANWVQQKPGQAFRGLIGGTNKRAPGVPA |
| | RFSGSLLGGKAALTLSGAQPEDEAEYYCALWYSNLWVFGGGTKLTVL (SEQ ID NO: 113) |
| 161F8_LC | QAVVTQEPSLTVSPGGTVTLTCRSSTGAVTTSNYANWVQQKPGQAPRGLIGGTNKRAPGTPA |
| | RFSGSLLGGKAALTLSGVQPEDEAEYYCALWYSNLWVFGGGTKLTVL (SEQ ID NO: 114) |
| 161G1_LC | QAVVTQEPSLTVSPGGTVTLTCRSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 115) |
| 161H2_LC | QAVVTQEPSLTVSPGGTVTLTCGSSTGAVTTSNYANWVQQKPGQAPRGLIGGTNKRAPGVPA |
| | RFSGSLLGGKAALTLSGAQPEDEAEYYCALWYSNLWVFGGGTKLTVL (SEQ ID NO: 116) |
| 162B10_LC | QAVVTQEPSLTVSPGGTVTLTCGSSTGAVTTSNYANWVQQKPGQAPRGLIGGTNKRAPGVPA |
| | RFSGSLLGGKAALTLSGAQPEDEAEYYCALWYSNLWVFGGGTKLTVL (SEQ ID NO: 117) |
| 162B12_LC | QAVVTQEPSLTVSPGGTVTLTCRSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 118) |
| 162D3_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 119) |
| 162D11_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 120) |
| 162G12_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 121) |
| 160D12_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 122) |
| 160E2_LC | QAVVTQEPSLTVSPGGTVTLTCGSSTGAVTTSNYANWVQQKPGQAPRGLIGGTNKRAPGVPA |
| | RFSGSLLGGKAALTLSGAQPEDEAEYYCALWYSNLWVFGGGTKLTVL (SEQ ID NO: 123) |
| 160E12_LC | QAVVTQEPSLTVSPGGTVTLTCGSSTGAVTTSNYANWVQQKPGQAPRGLIGGTNKRAPGTPA |
| | RFSGSLLGGKAALTLSGVQPEDEAEYYCALWYSNLWVFGGGTKLTVL (SEQ ID NO: 124) |
| 160F2_LC | QAVVTQEPSLTVSPGGTVTLTCGSSTGAVTTSNYANWVQQKPGQAPRGLIGGTNKRAPGVPA |
| | RFSGSLLGDKAALTLSGAQPEDEAEYYCALWYSNLWVFGGGTKLTVL (SEQ ID NO: 125) |
| 160F7_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 126) |
| 161E8_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 127) |
| 162C9_LC | QAVVTQEPSLTVSPGGTVTLTCGSSTGAVTTSNYANWVQQKPGQAFRGLIGGTNKRAPGVPA |
| | RFSGSLLGGKAALTLSGAQPEDEAEYYCALWYSNLWVFGGGTKLTVL (SEQ ID NO: 128) |
| 162D1_LC | QAVVTQEPSLTVSPGGTVTLTCRSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 129) |
| 162G7_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 130) |
| 162G11_LC | QAVVTQEPSLTVSPGGTVTLTCGSSTGAVTTSNYANWVQQKPGQAPRGLIGGTNKRAPGVPA |
| | RFSGSLLGGKAALTLSGAQPEDEAEYYCALWYSNLWVFGGGTKLTVL (SEQ ID NO: 131) |
| 162H12_LC | QAVVTQEPSLTVSPGGTVTLTCGSSTGAVTTSNYANWVQQKPGQAPRGLIGGTNKRAPGTPA |
| | RFSGSLLGGKAALTLSGAQPEDEAEYYCALWYSNLWVFGGGTKLTVL (SEQ ID NO: 132) |

TABLE 7
Anti-CD3 scFv sequences (VH-linker-VL)

| Clone ID | Sequence |
|---|---|
| 1B4 | EVKLVESGGGLVQPGGSLKLSCAASGFTFNTYAMNWVRQAPGKGLEWVGRIRSKYNNYAT |
| | YYADSVKDRFTISRDDSQNILYLQMNNLRTEDTAVYYCVRHGNFGNSYVSWFAYWGQGTL |
| | VTVSSGGGGSGGGGSGGGGSAQAFVTQEPSLTVSPGGTVTLTCGSSTGAVTTSNYANWVQQ |
| | KPGQAPRGLIGGTNKRAPGVPARFSGSLIGDKAALTITGAQPEDEAEYYCALWYSNLWVFGG |
| | GTKLTVL (SEQ ID NO: 133) |
| 1B10 | EVKLLESGGGLVQPGGSLKLSCAASGFTFNTYAMNWVRQAPGKGLEWVGRIRSKANNYAT |
| | YYADSVKGRFTISRDDSKNTLYLQMNNLKAEDTAVYYCVRHGNFGNSYVSWFAYWGQGTL |
| | VTVSSGGGGSGGGGSGGGGSAEAVVTQEPSLTVSPGGTVTLTCGSSTGAVTTSNYANWVQQ |
| | KPGQAPRGLIGGTNKRAPGTPARFSGSLLGDKAALTITGAQPEDEAEYYCALWYSNLWVFGG |
| | GTKLTVL (SEQ ID NO: 134) |
| 4A4 | EVKLVESGGGLVQPGGSLKLSCAASGFTFNTYAMNWVRQAPGKGLEWVGRIRSKYNNYAT |
| | YYADPVKGRFTISRDDAQNTLYLQMNNLKAEDTAVYYCVRHGNFGNSYVSWFAYWGQGTL |
| | VTVSSGGGGSGGGGSGGGGSAEAVVTQEPSLTVSPGGTVTLTCRSSTGAVTTSNYANWVQQ |
| | KPGQAFRGLIGGTNKRAPGVPARFSGSLLGDKAALTIAGAQPEDEAEYYCALWYSNLWVFG |
| | GGTKLTVL (SEQ ID NO: 135) |
| 4A5 | EVQLVESGGGLVQPGGSLKLSCAASGFTFNTYAMNWVRQAPGKGLEWVGRIRSKYNNYAT |
| | YYADSVKDRFTISRDDAKNTLYLQMNNLRAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRASGVPARFSGSLIGDKAALTIAGAQPEDEAEYYCALWYSNLWVFGG |
| | GTKLTVL (SEQ ID NO: 136) |
| 4A9 | EVKLVESGGGLVQPGGSLKLSCAASGFTFNTYAMNWVRQAPGKGLEWVGRIRSKYNNYAT |
| | YYADSVKGRFTISRDDSKNILYLQMNNLKAEDTAVYYCVRHGNFGNSYVSWFAYWGQGTL |
| | VTVSSGGGGSGGGGSGGGGSAQAVVTQEPSLTVSPGGTVTLTCRSSTGAVTTSNYANWVQQ |
| | KPGQAPRGLIGGTNKRAPGTPARFSGSLIGDKAALTITGVQPEDEAEYYCALWYSNLWVFGG |
| | GTKLTVL (SEQ ID NO: 137) |
| 4A10 | EVKLVESGGGLVQPGGSLKLSCAASGFTFNTYAMNWVRQAPGKGLEWVGRIRSKYNNYAT |
| | YYADSVKDRFTISRDDSKNTLYLQMNNLRAEDTAVYYCVRHGNFGNSYVSWFAYWGQGTL |
| | VTVSSGGGGSGGGGSGGGGSAEAVVTQEPSLTVSPGGTVTLTCRSSTGAVTTSNYANWVQQ |
| | KPGQAFRGLIGGTNKRAPGVPARFSGSLIGDKAALTITGAQPEDEAEYYCALWYSNLWVFGG |
| | GTKLTVL (SEQ ID NO: 138) |
| 4A10.1 | EVKLVESGGGLVQPGGSLKLSCAASGFTFNTYAMNWVRQAPGKGLEWVGRIRSKYNNYAT |
| | YYADSVKDRFTISRDDSKNTLYLQMNNLRAEDTAVYYCVRHGNFGNSYVSWFAYWGQGTL |
| | VTVSSGGGGSGGGGSGGGGSAEAVVTQEPSLTVSPGGTVTLTCRSSTGAVTTSNYANWVQQ |
| | KPGQAPRGLIGGTNKRAPGVPARFSGSLLGGKAALTLSGAQPEDEAEYYCALWYSNLWVFG |
| | GGTKLTVL (SEQ ID NO: 139) |
| 4A11 | EVKLVESGGGLVQPGGSLKLSCAASGFTFNTYAMNWVRQAPGKGLEWVGRIRSKYNNYAT |
| | YYAASVKDRFTISRDDSKNTLYLQMNNLRAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRAPGVPARFSGSLLGGKAALTITGAQPEDEAEYYCALWYSNLWVFG |
| | GGTKLTVL (SEQ ID NO: 140) |
| 4A11.1 | EVKLVESGGGLVQPGGSLKLSCAASGFTFNTYAMNWVRQAPGKGLEWVGRIRSKYNNYAT |
| | YYADSVKDRFTISRDDSKNTLYLQMNNLRAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGVPARFSGSLLGGKAALTLSGAQPEDEAEYYCALWYSNLWVFG |
| | GGTKLTVL (SEQ ID NO: 141) |
| 4A12 | EVQLVESGGGLVQPGGSLKLSCAASGFTFNTYAMNWVRQAPGKGLEWVGRIRSKYNNYAT |
| | YYADSVKDRFTISRDDAKNTLYLQMNNLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRAPGVPARFSGSLLGDKAALTITGAQPEDEAEYYCALWYSNLWVFG |
| | GGTKLTVL (SEQ ID NO: 142) |
| 4A12.1 | EVQLVESGGGLVQPGGSLKLSCAASGFTFNTYAMNWVRQAPGKGLEWVGRIRSKYNNYAT |
| | YYADSVKDRFTISRDDAKNTLYLQMNNLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGVPARFSGSLLGGKAALTLSGAQPEDEAEYYCALWYSNLWVFG |
| | GGTKLTVL (SEQ ID NO: 143) |
| 4B9 | EVQLLESGGGLVQPGGSLKLSCAASGFTFNTYAMNWVRQAPGKGLEWVGRIRSKANNYAT |
| | YYADSVKGRFTISRDDSKNILYLQMNSLRAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRAPGVPARFSGSLIGGKAALTIAGAQPEDEAEYYCALWYSNLWVFGG |
| | GTKLTVL (SEQ ID NO: 144) |
| 4B9.1 | EVQLLESGGGLVQPGGSLKLSCAASGFTFNTYAMNWVRQAPGKGLEWVGRIRSKANNYAT |
| | YYADSVKGRFTISRDDSKNILYLQMNSLRAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGVPARFSGSLLGGKAALTLAGAQPEDEAEYYCALWYSNLWVFG |
| | GGTKLTVL (SEQ ID NO: 145) |
| 4B12 | EVKLVESGGGLVQPGGSLKLSCAASGFTFNTYAMNWVRQAPGKGLEWVGRIRSKYNNYAT |
| | YYADSVKDRFTISRDDSKNTLYLQMNNLKAEDTAVYYCVRHGNFGNSYVSWFAYWGQGTL |
| | VTVSSGGGGSGGGGSGGGGSAEAVVTQEPSLTVSPGGTVTLTCRSSTGAVTTSNYANWVQQ |
| | KPGQAFRGLIGGTNKRAPGVPARFSGSLIGDKAALTITGAQPEDEAEYYCALWYSNLWVFGG |
| | GTKLTVL (SEQ ID NO: 146) |
| 4B12.1 | EVKLVESGGGLVQPGGSLKLSCAASGFTFNTYAMNWVRQAPGKGLEWVGRIRSKYNNYAT |
| | YYADSVKDRFTISRDDSKNTLYLQMNNLKAEDTAVYYCVRHGNFGNSYVSWFAYWGQGTL |
| | VTVSSGGGGSGGGGSGGGGSAEAVVTQEPSLTVSPGGTVTLTCRSSTGAVTTSNYANWVQQ |
| | KPGQAPRGLIGGTNKRAPGVPARFSGSLLGGKAALTLSGAQPEDEAEYYCALWYSNLWVFG |
| | GGTKLTVL (SEQ ID NO: 147) |
| 4C7 | EVRLVESGGGLVQPGGSLKLSCAASGFTFNTYAMNWVRQAPGKGLEWVGRIRSKTNNYATY |
| | YADSVKDRFTISRDDAQNILYLQMNNLRAEDTAVYYCVRHGNFGNSYVSWFAYWGQGTLV |
| | TVSSGGGGSGGGGSGGGGSAQAVVTQEPSLTVSPGGTVTLTCGSSTGAVTTSNYANWVQQK |
| | PGQAPRGLIGGTNKRAPGVPARFSGSLIGDKAALTIAGAQPEDEAEYYCALWYSNLWVFGGG |
| | TKLTVL (SEQ ID NO: 148) |
| 4C12 | EVKLVESGGGLVQPGGSLKLSCAASGFTFNTYAMNWVRQAPGKGLEWVGRIRSKSNNYATY |
| | YAASVKDRFTISRDDSKNSLYLQMNNLKAEDTAVYYCVRHGNFGNSYVSWFAYWGQGTLV |
| | TVSSGGGGSGGGGSGGGGSAQAVVTQEPSLTVSPGGTVTLTCGSSTGAVTNSNYANWVQQK |
| | PGQAFRGLIGGTNKRAPGTPARFSGSLLGGKAALTIAGVQPEDEAEYYCALWYSNLWVFGG |
| | GTKLTVL (SEQ ID NO: 149) |
| 4D4 | EVKLVESGGGLVQPGGSLKLSCAASGFTFNTYAMNWVRQAPGKGLEWVGRIRSKYNNYAT |
| | YYADSVKGRFTISRDDSKNTLYLQMNNLKTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPWTPARFSGSLIGDKAALTIAGAQPEDEAEYYCALWYSNLWVFGG |
| | GTKLTVL (SEQ ID NO: 150) |
| 4D6 | EVKLVESGGGLVQPGGSLKLSCAASGFTFNTYAMNWVRQAPGKGLEWVGRIRSKSNNYATY |
| | YADSVKDRFTISRDDSKNILYLQMNNLKAEDTAVYYCVRHGNFGNSYVSWFAYWGQGTLV |
| | TVSSGGGGSGGGGSGGGGSAEAVVTQEPSLTVSPGGTVTLTCGSSTGAVTTSNYANWVQQK |
| | PGQAFRGLIGGTNKRAPGTPARFSGSLIGGKAALTIAGAQPEDEAEYYCALWYSNLWVFGGG |
| | TKLTVL (SEQ ID NO: 151) |
| 4D6.1 | EVKLVESGGGLVQPGGSLKLSCAASGFTFNTYAMNWVRQAPGKGLEWVGRIRSKSNNYATY |
| | YADSVKDRFTISRDDSKNILYLQMNNLKAEDTAVYYCVRHGNFGNSYVSWFAYWGQGTLV |
| | TVSSGGGGSGGGGSGGGGSAEAVVTQEPSLTVSPGGTVTLTCGSSTGAVTTSNYANWVQQK |
| | PGQAPRGLIGGTNKRAPGTPARFSGSLLGGKAALTLAGAQPEDEAEYYCALWYSNLWVFGG |
| | GTKLTVL (SEQ ID NO: 152) |
| 4D7 | EVKLVESGGGLVQPGGSLKLSCAASGFTFNTYAMNWVRQAPGKGLEWVGRIRSKYNNYAT |
| | YYADSVKDRFTISRDDAQNILYLQMNNLKTEDTAVYYCVRHGNFGNSYVSWFAYWGQGTL |
| | VTVSSGGGGSGGGGSGGGGSAEAVVTQEPSLTVSPGGTVTLTCGSSTGAVTTSNYANWVQQ |
| | KPGQAFRGLIGGTNKRAPGVPARFSGSLIGDKAALTLAGAQPEDEAEYYCALWYSNLWVFG |
| | GGTKLTVL (SEQ ID NO: 153) |
| 4D9 | EVKLVESGGGLVQPGGSLKLSCAASGFTFNTYAMNWVRQAPGKGLEWVGRIRSKYNNYAT |
| | YYADSVKGRFTISRDDSQSILYLQMNNLRAEDTAVYYCVRHGNFGNSYVSWFAYWGQGTL |
| | VTVSSGGGGSGGGGSGGGGSAEAVVTQEPSLTVSPGGTVTLTCGSSTGAVTTSNYANWVQQ |
| | KPGQAFRGLIGGTNKRAPGVPARFSGSLIGDKAALTITGAQPEDEAEYYCALWYSNLWVFGG |
| | GTKLTVL (SEQ ID NO: 154) |
| 4E7 | EVKLVESGGGLVQPGGSLKLSCAASGFTFNTYAMNWVRQAPGKGLEWVGRIRSKYNNYAT |
| | YYADSVKDRFTISRDDSQNILYLQMNNLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGVPARFSGSLIGGKAALTITGVQPEDEAEYYCALWYSNLWVFGG |
| | GTKLTVL (SEQ ID NO: 155) |
| 4F5 | EVKLVESGGGLVQPGGSLKLSCAASGFTFNTYAMNWVRQAPGKGLEWVGRIRSKYNNYAT |
| | YYADPVKGRFTISRDDAKNTLYLQMNSLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRAPGVPARFSGSLIGDKAALTITGAQPEDEAEYYCALWYSNLWVFGG |
| | GTKLTVL (SEQ ID NO: 156) |
| 4F11 | EVKLVESGGGLVQPGGSLKLSCAASGFTFNTYAMNWVRQAPGKGLEWVGRIRSKYNNYAT |
| | YYADSVKDRFTISRDDSKSTLYLQMNNLRTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRAPGTPARFSGSLIGDKAALTITGAQPEDEAEYYCALWYSNLWVFGG |
| | GTKLTVL (SEQ ID NO: 157) |
| 4G1 | EVKLVESGGGLVQPGGSLKLSCAASGFTFNTYAMNWVRQAPGKGLEWVGRIRSKYNNYAT |
| | YYADSVKGRFTISRDDAQNILYLQMNNLKTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRAPGTPARFSGSLLGDKAALTITGAQPEDEAEYYCALWYSNLWVFGG |
| | GTKLTVL (SEQ ID NO: 158) |
| 4G2 | EVKLLESGGGLVQPGGSLKLSCAASGFTFNTYAMNWVRQAPGKGLEWVGRIRSKYNNYAT |
| | VTVSSGGGGSGGGGSGGGGSAEAVVTQEPSLTVSPGGTVTLTCGSSTGAVTTSNYANWVQQ |
| | KPGQAFRGLIGGTNKRAPGTPARFSGSLIGGKAALTITGAQPEDEAEYYCALWYSNLWVFGG |
| | GTKLTVL (SEQ ID NO: 159) |
| 160C9 | EVKLVESGGGLVQPGGSLKLSCAASGFTFNTYAMNWVRQAPGKGLEWVARIRSKYNNYAT |
| | YYADSVKDRFTISRDDAKNTLYLQMNNLRTEDTAVYYCVRHGNFGNSYVSWFAYWGQGTL |
| | VTVSSGGGGSGGGGSGGGGSAQAVVTQEPSLTVSPGGTVTLTCRSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 160) |
| 160_D10 | EVQLVESGGGLVQPGGSLKLSCAASGFTFNTYAMNWVRQAPGKGLEWVGRIRSKYNNYAT |
| | YYADSVKGRFTISRDDAKSTLYLQMNSLRTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRAPGVPARFSGSLLGGKAALTLSGAQPEDEAEYYCALWYSNLWVFG |
| | GGTKLTVL (SEQ ID NO: 161) |
| 160F1 | EVQLLESGGGLVQPGGSLKLSCAASGFTFNTYAMNWVRQAPGKGLEWVARIRSKYNNYAT |
| | YYADSVKDRFTISRDDSQNILYLQMNNLKTEDTAVYYCVRHGNFGNSYVSWFAYWGQGTL |
| | VTVSSGGGGSGGGGSGGGGSAQAVVTQEPSLTVSPGGTVTLTCRSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 162) |
| 160H4 | EVKLVESGGGLVQPGGSLKLSCAASGFTFNTYAMNWVRQAPGKGLEWVARIRSKYNNYAT |
| | YYADSVKDRFTISRDDSKNTLYLQMNSLKTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 163) |
| 161A8 | EVQLVESGGGLVQPGGSLKLSCAASGFTFNTYAMNWVRQAPGKGLEWVARIRSKYNNYAT |
| | YYADSVKDRFTISRDDSKNILYLQMNSLRTEDTAVYYCVRHGNFGNSYVSWFAYWGQGTLV |
| | TVSSGGGGSGGGGSGGGGSAQAVVTQEPSLTVSPGGTVTLTCGSSTGAVTTSNYANWVQQK |
| | PGQAPRGLIGGTNKRAPGTPARFSGSLLGGKAALTLSGAQPEDEAEYYCALWYSNLWVFGG |
| | GTKLTVL (SEQ ID NO: 164) |
| 161B4 | EVQLVESGGGLVQPGGSLKLSCAASGFTFNTYAMNWVRQAPGKGLEWVGRIRSKYNNYAT |
| | YYADSVKDRFTISRDDAQNILYLQMNNLKTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 165) |
| 161E6 | EVQLVESGGGLVQPGGSLKLSCAASGFTFNTYAMNWVRQAPGKGLEWVGRIRSKYNNYAT |
| | YYADSVKGRFTISRDDSKSSLYLQMNSLKAEDTAVYYCVRHGNFGNSYVSWFAYWGQGTL |
| | VTVSSGGGGSGGGGSGGGGSAQAVVTQEPSLTVSPGGTVTLTCRSSTGAVTTSNYANWVQQ |
| | KPGQAFRGLIGGTNKRAPGVPARFSGSLLGGKAALTLSGAQPEDEAEYYCALWYSNLWVFG |
| | GGTKLTVL (SEQ ID NO: 166) |
| 161F8 | EVKLVESGGGLVQPGGSLKLSCAASGFTFNTYAMNWVRQAPGKGLEWVGRIRSKYNNYAT |
| | YYADSVKGRFTISRDDSKNILYLQMNNLKAEDTAVYYCVRHGNFGNSYVSWFAYWGQGTL |
| | VTVSSGGGGSGGGGSGGGGSAQAVVTQEPSLTVSPGGTVTLTCRSSTGAVTTSNYANWVQQ |
| | KPGQAPRGLIGGTNKRAPGTPARFSGSLLGGKAALTLSGVQPEDEAEYYCALWYSNLWVFG |
| | GGTKLTVL (SEQ ID NO: 167) |
| 161G1 | EVKLVESGGGLVQPGGSLKLSCAASGFTFNTYAMNWVRQAPGKGLEWVGRIRSKYNNYAT |
| | YYADSVKGRFTISRDDSQNILYLQMNNLRTEDTAVYYCVRHGNFGNSYVSWFAYWGQGTL |
| | VTVSSGGGGSGGGGSGGGGSAQAVVTQEPSLTVSPGGTVTLTCRSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 168) |
| 161H2 | EVKLVESGGGLVQPGGSLKLSCAASGFTFNTYAMNWVRQAPGKGLEWVARIRSKYNNYAT |
| | YYADSVKDRFTISRDDAKNILYLQMNNLRTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGVPARFSGSLLGGKAALTLSGAQPEDEAEYYCALWYSNLWVFG |
| | GGTKLTVL (SEQ ID NO: 169) |
| 162B10 | EVQLVESGGGLVQPGGSLKLSCAASGFTFNTYAMNWVRQAPGKGLEWVGRIRSKYNNYAT |
| | VTVSSGGGGSGGGGSGGGGSAQAVVTQEPSLTVSPGGTVTLTCGSSTGAVTTSNYANWVQQ |
| | KPGQAPRGLIGGTNKRAPGVPARFSGSLLGGKAALTLSGAQPEDEAEYYCALWYSNLWVFG |
| | GGTKLTVL (SEQ ID NO: 170) |
| 162B12 | EVKLVESGGGLVQPGGSLKLSCAASGFTFNTYAMNWVRQAPGKGLEWVGRIRSKYNNYAT |
| | YYADSVKDRFTISRDDSKNILYLQMNNLRTEDTAVYYCVRHGNFGNSYVSWFAYWGQGTL |
| | VTVSSGGGGSGGGGSGGGGSAQAVVTQEPSLTVSPGGTVTLTCRSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 171) |
| 162D3 | EVQLLESGGGLVQPGGSLKLSCAASGFTFNTYAMNWVRQAPGKGLEWVGRIRSKYNNYAT |
| | YYADSVKGRFTISRDDSQNTLYLQMNNLKTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 172) |
| 162D11 | EVKLVESGGGLVQPGGSLKLSCAASGFTFNTYAMNWVRQAPGKGLEWVGRIRSKYNNYAT |
| | YYADPVKGRFTISRDDSKSTLYLQMNNLRTEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 173) |
| 162G12 | EVQLVESGGGLVQPGGSLKLSCAASGFTFNTYAMNWVRQAPGKGLEWVGRIRSKYNNYAT |
| | YYADSVKDRFTISRDDAKSTLYLQMNNLRAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 174) |
| 160D12 | EVQLVESGGGLVQPGGSLKLSCAASGFTFNTYAMNWVRQAPGKGLEWVGRIRSKYNNYAT |
| | YYADSVKGRFTISRDDSQSTLYLQMNSLRAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 175) |
| 160E2 | EVQLVESGGGLVQPGGSLKLSCAASGFTFNTYAMNWVRQAPGKGLEWVARIRSKYNNYAT |
| | YYADSVKGRFTISRDDSKNTLYLQMNNLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGVPARFSGSLLGGKAALTLSGAQPEDEAEYYCALWYSNLWVFG |
| | GGTKLTVL (SEQ ID NO: 176) |
| 160E12 | EVQLLESGGGLVQPGGSLKLSCAASGFTFNTYAMNWVRQAPGKGLEWVGRIRSKYNNYAT |
| | YYADSVKDRFTISRDDSKSTLYLQMNNLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGVQPEDEAEYYCALWYSNLWVFG |
| | GGTKLTVL (SEQ ID NO: 177) |
| 160F2 | EVKLVESGGGLVQPGGSLKLSCAASGFTFNTYAMNWVRQAPGKGLEWVSRIRSKYNNYATY |
| | YADPVKDRFTISRDDAQNTLYLQMNNLKAEDTAVYYCVRHGNFGNSYVSWFAYWGQGTLV |
| | TVSSGGGGSGGGGSGGGGSAQAVVTQEPSLTVSPGGTVTLTCGSSTGAVTTSNYANWVQQK |
| | PGQAPRGLIGGTNKRAPGVPARFSGSLLGDKAALTLSGAQPEDEAEYYCALWYSNLWVFGG |
| | GTKLTVL (SEQ ID NO: 178) |
| 160F7 | EVKLVESGGGLVQPGGSLKLSCAASGFTFNTYAMNWVRQAPGKGLEWVARIRSKYNNYAT |
| | YYADSVKGRFTISRDDSKNILYLQMNSLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | GGTKLTVL (SEQ ID NO: 179) |
| 161E8 | EVKLVESGGGLVQPGGSLKLSCAASGFTFNTYAMNWVRQAPGKGLEWVGRIRSKYNNYAT |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 180) |
| 162C9 | EVKLVESGGGLVQPGGSLKLSCAASGFTFNTYAMNWVRQAPGKGLEWVGRIRSKYNNYAT |
| | YYADSVKDRFTISRDDSQNTLYLQMNNLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAFRGLIGGTNKRAPGVPARFSGSLLGGKAALTLSGAQPEDEAEYYCALWYSNLWVFG |
| | GGTKLTVL (SEQ ID NO: 181) |
| 162D1 | EVQLVESGGGLVQPGGSLKLSCAASGFTFNTYAMNWVRQAPGKGLEWVGRIRSKYNNYAT |
| | YYADSVKGRFTISRDDAKSTLYLQMNNLKTEDTAVYYCVRHGNFGNSYVSWFAYWGQGTL |
| | VTVSSGGGGSGGGGSGGGGSAQAVVTQEPSLTVSPGGTVTLTCRSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 182) |
| 162G7 | EVKLVESGGGLVQPGGSLKLSCAASGFTFNTYAMNWVRQAPGKGLEWVARIRSKYNNYAT |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 183) |
| 162G11 | EVKLVESGGGLVQPGGSLKLSCAASGFTFNTYAMNWVRQAPGKGLEWVGRIRSKYNNYAT |
| | YYADSVKGRFTISRDDAKNSLYLQMNNLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGVPARFSGSLLGGKAALTLSGAQPEDEAEYYCALWYSNLWVFG |
| | GGTKLTVL (SEQ ID NO: 184) |
| 162H12 | EVKLVESGGGLVQPGGSLKLSCAASGFTFNTYAMNWVRQAPGKGLEWVGRIRSKYNNYAT |
| | YYADPVKDRFTISRDDSQNILYLQMNNLKAEDTAVYYCVRHGNFGNSYVSWFAYWGQGTL |
| | VTVSSGGGGSGGGGSGGGGSAQAVVTQEPSLTVSPGGTVTLTCGSSTGAVTTSNYANWVQQ |
| | KPGQAPRGLIGGTNKRAPGTPARFSGSLLGGKAALTLSGAQPEDEAEYYCALWYSNLWVFG |
| | GGTKLTVL (SEQ ID NO: 185) |

TABLE 8
LRRC15 sequences

| Description | Sequence |
|---|---|
| LRRC15 | MPLKHYLLLLVGCQAWGAGLAYHGC |
| human | PSECTCSRASQVECTGARIVAVPTP |
| (UniProt: | LPWNAMSLQILNTHITELNESPFLN |
| Q8TF66) | ISALIALRIEKNELSRITPGAFRNL |
| | GSLRYLSLANNKLQVLPIGLFQGLD |
| | SLESLLLSSNQLLQIQPAHFSQCSN |
| | LKELQLHGNHLEYIPDGAFDHLVGL |
| | TKLNLGKNSLTHISPRVFQHLGNLQ |
| | VLRLYENRLTDIPMGTFDGLVNLQE |
| | LALQQNQIGLLSPGLFHNNHNLQRL |
| | YLSNNHISQLPPSVFMQLPQLNRLT |
| | LFGNSLKELSPGIFGPMPNLRELWL |
| | YDNHISSLPDNVFSNLRQLQVLILS |
| | RNQISFISPGAFNGLTELRELSLHT |
| | NALQDLDGNVFRMLANLQNISLQNN |
| | RLRQLPGNIFANVNGLMAIQLQNNQ |
| | LENLPLGIFDHLGKLCELRLYDNPW |
| | RCDSDILPLRNWLLLNQPRLGTDTV |
| | PVCFSPANVRGQSLIIINVNVAVPS |
| | VHVPEVPSYPETPWYPDTPSYPDTT |
| | SVSSTTELTSPVEDYTDLTTIQVTD |
| | DRSVWGMTQAQSGLAIAAIVIGIVA |
| | LACSLAACVGCCCCKKRSQAVLMQM |
| | KAPNEC |
| | (SEQ ID NO: 186) |
| cyLRRC15 | MLAMPLKHYLLLLVGCQAWGAGLAY |
| A0A2K5UK | YGCPSECTCSRASQVECTGARIVAV |
| B6 Macaca | PTPLPWNAMSLQILNTHITELSESP |
| fascicularis | FLNISALIALRIEKNELSHIMPGAF |
| | RNLGSLRYLSLANNKLQVLPIGLFQ |
| | GLDSLESLLLSSNQLVQIQPAHFSQ |
| | CSNLKELQLHGNHLEYIPDGAFDHL |
| | VGLTKLNLGKNSLTHISPRVFQHLG |
| | NLQVLRLYENRLTDIPMGTFDGLVN |
| | LQELALQQNQIGLLSPGLFHNNHNL |
| | QRLYLSNNHISQLPPSIFMQLPQLN |
| | RLTLFGNSLKELSPGIFGPMPNLRE |
| | LWLYDNHITSLPDNVFSNLRQLQVL |
| | ILSRNQISFISPGAFNGLTELRELS |
| | LHTNALQDLDGNVFRMLANLQNISL |
| | QNNRLRQLPGNIFANVNGLMTIQLQ |
| | NNQLENLPLGIFDHLGKLCELRLYD |
| | NPWRCDSDILPLRNWLLLNQPRLGT |
| | DTVPVCFSPANVRGQSLIIINVNVA |
| | VPSVHVPEVPSYPETSWYPDTSSYP |
| | DTTSISSTTELTSPVEDYTDLTTIQ |
| | VTDDRSVWGMTQAQSGLAIAAIVIG |
| | IVALACSLAACIGCCCCKKRSQAVL |
| | MQMKAPNEC |
| | (SEQ ID NO: 187) |
| msLRRC15 | MPLKHYLLLLVSCQAWAAGLAYYGC |
| Q80X72_ | PSECTCSRASQVECTGAQIVAMPSP |
| MOUSE | LPWNAMSLQILNTHITELPEDKFLN |
| | ISALIALKMEKNELANIMPGAFRNL |
| | GSLRHLSLANNKLKNLPVRLFQDVN |
| | NLETLLLSNNQLVQIQPAQFSQFSN |
| | LKELQLYGNNLEYIPEGVFDHLVGL |
| | TKLNLGNNGFTHLSPRVFQHLGNLQ |
| | VLRLYENRLSDIPMGTFDALGNLQE |
| | LALQENQIGTLSPGLFHNNRNLQRL |
| | YLSNNHISHLPPGIFMQLPHLNKLT |
| | LFGNSLKELSPGVFGPMPNLRELWL |
| | YNNHITSLPDNAFSHLNQLQVLILS |
| | HNQLSYISPGAFNGLTNLRELSLHT |
| | NALQDLDGNVFRSLANLRNVSLQNN |
| | RLRQLPGSIFANVNGLMTIQLQNNN |
| | LENLPLGIFDHLGNLCELRLYDNPW |
| | RCDSNILPLHDWLILNRARLGTDTL |
| | PVCSSPASVRGQSLVIINVNFPGPS |
| | VQGPETPEVSSYPDTSSYPDSTSIS |
| | STTEITRSTDDDYTDLNTIEPIDDR |
| | NTWGMTDAQSGLAIAAIVIGIIALA |
| | CSLAACICCCCCKKRSQAVLMQMKA |
| | PNEC |
| | (SEQ ID NO: 188) |

TABLE 9
Miscellaneous Sequences

| Description | Sequence |
|---|---|
| Linker 1a | EPKSCDKTHT |
| | (SEQ ID NO: 189) |
| Linker 1b | EPKSCDGGSGGSGGSG |
| | (SEQ ID NO: 190) |
| Linker 1c | EPKSCDGGGGSGGGGS |
| | (SEQ ID NO: 191) |
| (G4S)1 linker | GGGGS |
| | (SEQ ID NO: 192) |
| (G4S)2 linker | GGGGSGGGGS |
| | (SEQ ID NO: 193) |
| (G4S)3 linker | GGGGSGGGGSGGGGSA |
| | (SEQ ID NO: 229) |
| Reduced Feg and | EPKSCDKTHTCPPCPAPEAAGA |
| compliment binding | (SEQ ID NO: 230) |
| Fc sequence | |
| Linker 1d | EPKSSDKTHT |
| | (SEQ ID NO: 241) |
| Reduced Fcg and | EPKSSDKTHTCPPCPAPEAAGA |
| compliment binding | (SEQ ID NO: 242) |
| Fc sequence | |

TABLE 10
Full length light chain sequences

| | Full length light |
|---|---|
| Clone ID | chain sequence |
| huLRRC15 LC | DIQMTQSPSSLSASVGDRVTITCRA |
| | SQDISNYLNWYQQKPGKAPKFLIYY |
| | TSRLHSGVPSRFSGSGSGTDYTLTI |
| | SSLQPEDFATYYCQQGEALPWTFGQ |
| | GTKVEIKRTVAAPSVFIFPPSDEQL |
| | KSGTASVVCLLNNFYPREAKVQWKV |
| | DNALQSGNSQESVTEQDSKDSTYSL |
| | SSTLTLSKADYEKHKVYACEVTHQG |
| | LSSPVTKSFNRGEC |
| | (SEQ ID NO: 194) |

TABLE 11
Full length heavy chain sequences, Type 2 format

| | Full length heavy chain |
|---|---|
| Clone ID | sequence (Type 2 HC) |
| 160C9_T2a | QVQLVQSGAEVKKPGASVKVSCKASGYKFT |
| | SYWIEWVRQAPGQGLEWIGEILPGSDTTNY |
| | NEKFKDRVTFTSDTSTSTVYMELSSLRSED |
| | TAVYYCARDRGNYRAWFGYWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGA |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGG |
| | SEVKLVESGGGLVQPGGSLKLSCAASGFTF |
| | NTYAMNWVRQAPGKGLEWVARIRSKYNNYA |
| | TYYADSVKDRFTISRDDAKNTLYLQMNNLR |
| | TEDTAVYYCVRHGNFGNSYVSWFAYWGQGT |
| | LVTVSSGGGGSGGGGSGGGGSAQAVVTQEP |
| | SLTVSPGGTVTLTCRSSTGAVTTSNYANWV |
| | QQKPGQAPRGLIGGTNKRAPGTPARFSGSL |
| | LGGKAALTLSGAQPEDEAEYYCALWYSNLW |
| | VFGGGTKLTVL (SEQID NO: 195) |
| 1B4_T2a | QVQLVQSGAEVKKPGASVKVSCKASGYKFT |
| | SYWIEWVRQAPGQGLEWIGEILPGSDTTNY |
| | NEKFKDRVTFTSDTSTSTVYMELSSLRSED |
| | TAVYYCARDRGNYRAWFGYWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGA |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGG |
| | SEVKLVESGGGLVQPGGSLKLSCAASGFTF |
| | NTYAMNWVRQAPGKGLEWVGRIRSKYNNYA |
| | TYYADSVKDRFTISRDDSQNILYLQMNNLR |
| | TEDTAVYYCVRHGNFGNSYVSWFAYWGQGT |
| | LVTVSSGGGGSGGGGSGGGGSAQAFVTQEP |
| | SLTVSPGGTVTLTCGSSTGAVTTSNYANWV |
| | QQKPGQAPRGLIGGTNKRAPGVPARFSGSL |
| | IGDKAALTITGAQPEDEAEYYCALWYSNLW |
| | VFGGGTKLTVL (SEQID NO: 196) |
| 4G2_T2a | QVQLVQSGAEVKKPGASVKVSCKASGYKFT |
| | SYWIEWVRQAPGQGLEWIGEILPGSDTTNY |
| | NEKFKDRVTFTSDTSTSTVYMELSSLRSED |
| | TAVYYCARDRGNYRAWFGYWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGA |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGG |
| | SEVKLLESGGGLVQPGGSLKLSCAASGFTF |
| | NTYAMNWVRQAPGKGLEWVGRIRSKYNNYA |
| | TYYADSVKGRFTISRDDAKSILYLQMNNLK |
| | TEDTAVYYCVRHGNFGNSYVSWFAYWGQGT |
| | LVTVSSGGGGSGGGGSGGGGSAEAVVTQEP |
| | SLTVSPGGTVTLTCGSSTGAVTTSNYANWV |
| | QQKPGQAFRGLIGGTNKRAPGTPARFSGSL |
| | IGGKAALTITGAQPEDEAEYYCALWYSNLW |
| | VFGGGTKLTVL (SEQID NO: 197) |
| 1B10_T2a | QVQLVQSGAEVKKPGASVKVSCKASGYKFT |
| | SYWIEWVRQAPGQGLEWIGEILPGSDTTNY |
| | NEKFKDRVTFTSDTSTSTVYMELSSLRSED |
| | TAVYYCARDRGNYRAWFGYWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGA |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGG |
| | SEVKLLESGGGLVQPGGSLKLSCAASGFTF |
| | NTYAMNWVRQAPGKGLEWVGRIRSKANNYA |
| | TYYADSVKGRFTISRDDSKNTLYLQMNNLK |
| | AEDTAVYYCVRHGNFGNSYVSWFAYWGQGT |
| | LVTVSSGGGGSGGGGSGGGGSAEAVVTQEP |
| | SLTVSPGGTVTLTCGSSTGAVTTSNYANWV |
| | QQKPGQAPRGLIGGTNKRAPGTPARFSGSL |
| | LGDKAALTITGAQPEDEAEYYCALWYSNLW |
| | VFGGGTKLTVL |
| | (SEQ ID NO: 233) |
| 160C9_T2c | QVQLVQSGAEVKKPGASVKVSCKASGYKFT |
| | SYWIEWVRQAPGQGLEWIGEILPGSDTTNY |
| | NEKFKDRVTFTSDTSTSTVYMELSSLRSED |
| | TAVYYCARDRGNYRAWFGYWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGA |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGG |
| | GSGGSGGSEVKLVESGGGLVQPGGSLKLSC |
| | AASGFTFNTYAMNWVRQAPGKGLEWVARIR |
| | SKYNNYATYYADSVKDRFTISRDDAKNTLY |
| | LQMNNLRTEDTAVYYCVRHGNFGNSYVSWF |
| | AYWGQGTLVTVSSGGGGSGGGGSGGGGSAQ |
| | AVVTQEPSLTVSPGGTVTLTCRSSTGAVTT |
| | SNYANWVQQKPGQAPRGLIGGTNKRAPGTP |
| | ARFSGSLLGGKAALTLSGAQPEDEAEYYCA |
| | LWYSNLWVFGGGTKLTVL |
| | (SEQ ID NO: 198) |
| 1B4_T2c | QVQLVQSGAEVKKPGASVKVSCKASGYKFT |
| | SYWIEWVRQAPGQGLEWIGEILPGSDTTNY |
| | NEKFKDRVTFTSDTSTSTVYMELSSLRSED |
| | TAVYYCARDRGNYRAWFGYWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGA |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGG |
| | GSGGSGGSEVKLVESGGGLVQPGGSLKLSC |
| | AASGFTFNTYAMNWVRQAPGKGLEWVGRIR |
| | SKYNNYATYYADSVKDRFTISRDDSQNILY |
| | LQMNNLRTEDTAVYYCVRHGNFGNSYVSWF |
| | AYWGQGTLVTVSSGGGGSGGGGSGGGGSAQ |
| | AFVTQEPSLTVSPGGTVTLTCGSSTGAVTT |
| | SNYANWVQQKPGQAPRGLIGGTNKRAPGVP |
| | ARFSGSLIGDKAALTITGAQPEDEAEYYCA |
| | LWYSNLWVFGGGTKLTVL |
| | (SEQ ID NO: 199) |
| 4G2_T2c | QVQLVQSGAEVKKPGASVKVSCKASGYKFT |
| | SYWIEWVRQAPGQGLEWIGEILPGSDTTNY |
| | NEKFKDRVTFTSDTSTSTVYMELSSLRSED |
| | TAVYYCARDRGNYRAWFGYWGQGTLVTVSS |
| | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGA |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGG |
| | GSGGSGGSEVKLLESGGGLVQPGGSLKLSC |
| | AASGFTFNTYAMNWVRQAPGKGLEWVGRIR |
| | SKYNNYATYYADSVKGRFTISRDDAKSILY |
| | LQMNNLKTEDTAVYYCVRHGNFGNSYVSWF |
| | AYWGQGTLVTVSSGGGGSGGGGSGGGGSAE |
| | AVVTQEPSLTVSPGGTVTLTCGSSTGAVTT |
| | SNYANWVQQKPGQAFRGLIGGTNKRAPGTP |
| | ARFSGSLIGGKAALTITGAQPEDEAEYYCA |
| | LWYSNLWVFGGGTKLTVL |
| | (SEQ ID NO: 200) |
| 1B10_T2c | QVQLVQSGAEVKKPGASVKVSCKASGYKFT |
| | SYWIEWVRQAPGQGLEWIGEILPGSDTTN |
| | YNEKFKDRVTFTSDTSTSTVYMELS |
| | SLRSEDTAVYYCARDRGNYRAWFGYWGQGT |
| | LVTVSSASTKGPSVFPLAPSSKSTSGGTAA |
| | LGCLVKDYFPEPVTVSWNSGALTSGVHTFP |
| | AVLQSSGLYSLSSVVTVPSSSLGTQTYICN |
| | VNHKPSNTKVDKKVEPKSCDKTHTCPPCPA |
| | PEAAGAPSVFLFPPKPKDTLMISRTPEVTC |
| | VVVDVSHEDPEVKFNWYVDGVEVHNAKTKP |
| | REEQYNSTYRVVSVLTVLHQDWLNGKEYKC |
| | KVSNKALPAPIEKTISKAKGQPREPQVYTL |
| | PPSRDELTKNQVSLTCLVKGFYPSDIAVEW |
| | ESNGQPENNYKTTPPVLDSDGSFFLYSKLT |
| | VDKSRWQQGNVFSCSVMHEALHNHYTQKSL |
| | SLSPGGGSGGSGGSEVKLLESGGGLVQPGG |
| | SLKLSCAASGFTFNTYAMNWVRQAPGKGLE |
| | WVGRIRSKANNYATYYADSVKGRFTISRDD |
| | SKNTLYLQMNNLKAEDTAVYYCVRHGNFGN |
| | SYVSWFAYWGQGTLVTVSSGGGGSGGGGSG |
| | GGGSAEAVVTQEPSLTVSPGGTVTLTCGSS |
| | TGAVTTSNYANWVQQKPGQAPRGLIGGTNK |
| | RAPGTPARFSGSLLGDKAALTITGAQPEDE |
| | AEYYCALWYSNLWVFGGGTKLTVL |
| | (SEQ ID NO: 234) |

TABLE 12
Full length heavy chain sequences, Type 4 format

| Clone ID | Full length heavy chain sequence (Type 4 HC) |
|---|---|
| huLRRC15_ | QVQLVQSGAEVKKPGASVKVSCKASGYKFTSYWIEWVRQAPGQGLEWIGEILPG |
| VHv2_ | SDTTNYNEKFKDRVTFTSDTSTSTVYMELSSLRSEDTAVYYCARDRGNYRAWFG |
| Hole | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPEAAGAPSVFLFPPKPKDTLMISRTPEVTCVVVDVSHEDP |
| | EVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLSCAVKGFYPSDIAV |
| | EWESNGQPENNYKTTPPVLDSDGSFFLVSKLTVDKSRWQQGNVFSCSVMHEALH |
| | NHYTQKSLSLSPG (SEQ ID NO: 201) |
| huLRRC15_ | QVQLVQSGAEVKKPGASVKVSCKASGYKFTSYWIEWVRQAPGQGLEWIGEILPG |
| VHv2_ | SDTTNYNEKFKDRVTFTSDTSTSTVYMELSSLRSEDTAVYYCARDRGNYRAWFG |
| Hole_ss | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPEAAGAPSVFLFPPKPKDTLMISRTPEVTCVVVDVSHEDP |
| | EVKFNWYVDGVEVHNAKTKPREEQYNSTYRVVSVLTVLHQDWLNGKEYKCKVSN |
| | KALPAPIEKTISKAKGQPREPQVCTLPPSRDELTKNQVSLSCAVKGFYPSDIAVE |
| | WESNGQPENNYKTTPPVLDSDGSFFLVSKLTVDKSRWQQGNVFSCSVMHEALHN |
| | HYTQKSLSLSPG (SEQ ID NO: 231) |
| 160C9_T4h | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTEVKLVESGGGLVQPGGSLKLSCAASGFTFNTYAMNWVRQAPGK |
| | GLEWVARIRSKYNNYATYYADSVKDRFTISRDDAKNTLYLQMNNLRTEDTAVY |
| | YCVRHGNFGNSYVSWFAYWGQGTLVTVSSGGGGSGGGGSGGGGSAQAVVTQE |
| | PSLTVSPGGTVTLTCRSSTGAVTTSNYANWVQQKPGQAPRGLIGGTNKRAPGTPA |
| | RFSGSLLGGKAALTLSGAQPEDEAEYYCALWYSNLWVFGGGTKLTVLEPKSCDK |
| | THTCPPCPAPEAAGAPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALP |
| | APIEKTISKAKGQPREPQVYTLPPSRDELTKNQVSLWCLVKGFYPSDIAVEWESNG |
| | QPENNYKTTPPVLDSDGSFFLYSKLTVDKSRWQQGNVFSCSVMHEALHNHYTQK |
| | SLSLSPG (SEQ ID NO: 202) |
| 160C9_T4h_ | QVQLVQSGAEVKKPGASVKVSCKASGYKFTSYWIEWVRQAPGQGLEWIGEILPG |
| ss | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTEVKLVESGGGLVQPGGSLKLSCAASGFTFNTYAMNWVRQAPGK |
| | GLEWVARIRSKYNNYATYYADSVKDRFTISRDDAKNTLYLQMNNLRTEDTAVY |
| | YCVRHGNFGNSYVSWFAYWGQGTLVTVSSGGGGSGGGGSGGGGSAQAVVTQE |
| | PSLTVSPGGTVTLTCRSSTGAVTTSNYANWVQQKPGQAPRGLIGGTNKRAPGTPA |
| | RFSGSLLGGKAALTLSGAQPEDEAEYYCALWYSNLWVFGGGTKLTVLEPKSSDK |
| | THTCPPCPAPEAAGAPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALP |
| | APIEKTISKAKGQPREPQVYTLPPCRDELTKNQVSLWCLVKGFYPSDIAVEWESN |
| | GQPENNYKTTPPVLDSDGSFFLYSKLTVDKSRWQQGNVFSCSVMHEALHNRFTQ |
| | KSLSLSPG (SEQ ID NO: 235) |
| 1B4_T4h | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTEVKLVESGGGLVQPGGSLKLSCAASGFTFNTYAMNWVRQAPGK |
| | GLEWVGRIRSKYNNYATYYADSVKDRFTISRDDSQNILYLQMNNLRTEDTAVYY |
| | CVRHGNFGNSYVSWFAYWGQGTLVTVSSGGGGSGGGGSGGGGSAQAFVTQEPS |
| | LTVSPGGTVTLTCGSSTGAVTTSNYANWVQQKPGQAPRGLIGGTNKRAPGVPAR |
| | FSGSLIGDKAALTITGAQPEDEAEYYCALWYSNLWVFGGGTKLTVLEPKSCDKT |
| | HTCPPCPAPEAAGAPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAP |
| | IEKTISKAKGQPREPQVYTLPPSRDELTKNQVSLWCLVKGFYPSDIAVEWESNGQP |
| | ENNYKTTPPVLDSDGSFFLYSKLTVDKSRWQQGNVFSCSVMHEALHNHYTQKSL |
| | SLSPG (SEQ ID NO: 203) |
| 1B4_T4h_ss | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTEVKLVESGGGLVQPGGSLKLSCAASGFTFNTYAMNWVRQAPGK |
| | GLEWVGRIRSKYNNYATYYADSVKDRFTISRDDSQNILYLQMNNLRTEDTAVYY |
| | CVRHGNFGNSYVSWFAYWGQGTLVTVSSGGGGSGGGGSGGGGSAQAFVTQEPS |
| | LTVSPGGTVTLTCGSSTGAVTTSNYANWVQQKPGQAPRGLIGGTNKRAPGVPAR |
| | FSGSLIGDKAALTITGAQPEDEAEYYCALWYSNLWVFGGGTKLTVLEPKSSDKTH |
| | TCPPCPAPEAAGAPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPI |
| | EKTISKAKGQPREPQVYTLPPCRDELTKNQVSLWCLVKGFYPSDIAVEWESNGQP |
| | ENNYKTTPPVLDSDGSFFLYSKLTVDKSRWQQGNVFSCSVMHEALHNRFTQKSL |
| | SLSPG (SEQ ID NO: 236) |
| 4G2_T4h | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTEVKLLESGGGLVQPGGSLKLSCAASGFTFNTYAMNWVRQAPGKG |
| | LEWVGRIRSKYNNYATYYADSVKGRFTISRDDAKSILYLQMNNLKTEDTAVYYC |
| | VRHGNFGNSYVSWFAYWGQGTLVTVSSGGGGSGGGGSGGGGSAEAVVTQEPSL |
| | TVSPGGTVTLTCGSSTGAVTTSNYANWVQQKPGQAFRGLIGGTNKRAPGTPARF |
| | SGSLIGGKAALTITGAQPEDEAEYYCALWYSNLWVFGGGTKLTVLEPKSCDKTH |
| | TCPPCPAPEAAGAPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPI |
| | EKTISKAKGQPREPQVYTLPPSRDELTKNQVSLWCLVKGFYPSDIAVEWESNGQP |
| | ENNYKTTPPVLDSDGSFFLYSKLTVDKSRWQQGNVFSCSVMHEALHNHYTQKSL |
| | SLSPG (SEQ ID NO: 204) |
| 1B10_T4h_ss | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTEVKLLESGGGLVQPGGSLKLSCAASGFTFNTYAMNWVRQAPGKG |
| | LEWVGRIRSKANNYATYYADSVKGRFTISRDDSKNTLYLQMNNLKAEDTAVYY |
| | CVRHGNFGNSYVSWFAYWGQGTLVTVSSGGGGSGGGGSGGGGSAEAVVTQEPS |
| | LTVSPGGTVTLTCGSSTGAVTTSNYANWVQQKPGQAPRGLIGGTNKRAPGTPAR |
| | FSGSLLGDKAALTITGAQPEDEAEYYCALWYSNLWVFGGGTKLTVLEPKSSDKT |
| | HTCPPCPAPEAAGAPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAP |
| | IEKTISKAKGQPREPQVYTLPPCRDELTKNQVSLWCLVKGFYPSDIAVEWESNGQ |
| | PENNYKTTPPVLDSDGSFFLYSKLTVDKSRWQQGNVFSCSVMHEALHNRFTQKS |
| | LSLSPG (SEQ ID NO: 237) |
| 4D4_T4h | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTEVKLVESGGGLVQPGGSLKLSCAASGFTFNTYAMNWVRQAPGK |
| | GLEWVGRIRSKYNNYATYYADSVKGRFTISRDDSKNTLYLQMNNLKTEDTAVY |
| | YCVRHGNFGNSYVSWFAYWGQGTLVTVSSGGGGSGGGGSGGGGSAQAVVTQE |
| | PSLTVSPGGTVTLTCGSSTGAVTTSNYANWVQQKPGQAPRGLIGGTNKRAPWTP |
| | ARFSGSLIGDKAALTIAGAQPEDEAEYYCALWYSNLWVFGGGTKLTVLEPKSCD |
| | KTHTCPPCPAPEAAGAPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKAL |
| | PAPIEKTISKAKGQPREPQVYTLPPSRDELTKNQVSLWCLVKGFYPSDIAVEWESN |
| | GQPENNYKTTPPVLDSDGSFFLYSKLTVDKSRWQQGNVFSCSVMHEALHNHYTQ |
| | KSLSLSPG (SEQ ID NO: 205) |
| 160C9_T4d | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDGGGGSGGGGSEVKLVESGGGLVQPGGSLKLSCAASGFTFNTYAMNWV |
| | RQAPGKGLEWVARIRSKYNNYATYYADSVKDRFTISRDDAKNTLYLQMNNLRT |
| | EDTAVYYCVRHGNFGNSYVSWFAYWGQGTLVTVSSGGGGSGGGGSGGGGSAQ |
| | AVVTQEPSLTVSPGGTVTLTCRSSTGAVTTSNYANWVQQKPGQAPRGLIGGTNK |
| | RAPGTPARFSGSLLGGKAALTLSGAQPEDEAEYYCALWYSNLWVFGGGTKLTVL |
| | EPKSCDKTHTCPPCPAPEAAGAPSVFLFPPKPKDTLMISRTPEVTCVVVDVSHEDP |
| | EVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLWCLVKGFYPSDIAV |
| | EWESNGQPENNYKTTPPVLDSDGSFFLYSKLTVDKSRWQQGNVFSCSVMHEALH |
| | NHYTQKSLSLSPG (SEQ ID NO: 206) |
| 1B4_T4d | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDGGGGSGGGGSEVKLVESGGGLVQPGGSLKLSCAASGFTFNTYAMNWV |
| | RQAPGKGLEWVGRIRSKYNNYATYYADSVKDRFTISRDDSQNILYLQMNNLRTE |
| | DTAVYYCVRHGNFGNSYVSWFAYWGQGTLVTVSSGGGGSGGGGSGGGGSAQA |
| | FVTQEPSLTVSPGGTVTLTCGSSTGAVTTSNYANWVQQKPGQAPRGLIGGTNKR |
| | APGVPARFSGSLIGDKAALTITGAQPEDEAEYYCALWYSNLWVFGGGTKLTVLEP |
| | KSCDKTHTCPPCPAPEAAGAPSVFLFPPKPKDTLMISRTPEVTCVVVDVSHEDPEV |
| | KFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLWCLVKGFYPSDIAVEW |
| | ESNGQPENNYKTTPPVLDSDGSFFLYSKLTVDKSRWQQGNVFSCSVMHEALHNH |
| | YTQKSLSLSPG (SEQ ID NO: 207) |
| 4G2_T4d | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDGGGGSGGGGSEVKLLESGGGLVQPGGSLKLSCAASGFTFNTYAMNWV |
| | RQAPGKGLEWVGRIRSKYNNYATYYADSVKGRFTISRDDAKSILYLQMNNLKTE |
| | DTAVYYCVRHGNFGNSYVSWFAYWGQGTLVTVSSGGGGSGGGGSGGGGSAEA |
| | VVTQEPSLTVSPGGTVTLTCGSSTGAVTTSNYANWVQQKPGQAFRGLIGGTNKR |
| | APGTPARFSGSLIGGKAALTITGAQPEDEAEYYCALWYSNLWVFGGGTKLTVLEP |
| | KSCDKTHTCPPCPAPEAAGAPSVFLFPPKPKDTLMISRTPEVTCVVVDVSHEDPEV |
| | KFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLWCLVKGFYPSDIAVEW |
| | ESNGQPENNYKTTPPVLDSDGSFFLYSKLTVDKSRWQQGNVFSCSVMHEALHNH |
| | YTQKSLSLSPG (SEQ ID NO: 208) |
| 160C9_T4e | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDEVKLVESGGGLVQPGGSLKLSCAASGFTFNTYAMNWVRQAPGKGLEW |
| | VARIRSKYNNYATYYADSVKDRFTISRDDAKNTLYLQMNNLRTEDTAVYYCVR |
| | HGNFGNSYVSWFAYWGQGTLVTVSSGGGGSGGGGSGGGGSAQAVVTQEPSLTV |
| | SPGGTVTLTCRSSTGAVTTSNYANWVQQKPGQAPRGLIGGTNKRAPGTPARFSGS |
| | LLGGKAALTLSGAQPEDEAEYYCALWYSNLWVFGGGTKLTVLEPKSCDKTHTCP |
| | PCPAPEAAGAPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTI |
| | SKAKGQPREPQVYTLPPSRDELTKNQVSLWCLVKGFYPSDIAVEWESNGQPENN |
| | YKTTPPVLDSDGSFFLYSKLTVDKSRWQQGNVFSCSVMHEALHNHYTQKSLSLS |
| | PG (SEQ ID NO: 209) |
| 1B4_T4e | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDEVKLVESGGGLVQPGGSLKLSCAASGFTFNTYAMNWVRQAPGKGLEW |
| | VGRIRSKYNNYATYYADSVKDRFTISRDDSQNILYLQMNNLRTEDTAVYYCVRH |
| | GNFGNSYVSWFAYWGQGTLVTVSSGGGGSGGGGSGGGGSAQAFVTQEPSLTVS |
| | PGGTVTLTCGSSTGAVTTSNYANWVQQKPGQAPRGLIGGTNKRAPGVPARFSGS |
| | LIGDKAALTITGAQPEDEAEYYCALWYSNLWVFGGGTKLTVLEPKSCDKTHTCP |
| | PCPAPEAAGAPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTI |
| | SKAKGQPREPQVYTLPPSRDELTKNQVSLWCLVKGFYPSDIAVEWESNGQPENN |
| | YKTTPPVLDSDGSFFLYSKLTVDKSRWQQGNVFSCSVMHEALHNHYTQKSLSLS |
| | PG (SEQ ID NO: 210) |
| 4G2_T4e | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDEVKLLESGGGLVQPGGSLKLSCAASGFTFNTYAMNWVRQAPGKGLEW |
| | VGRIRSKYNNYATYYADSVKGRFTISRDDAKSILYLQMNNLKTEDTAVYYCVRH |
| | GNFGNSYVSWFAYWGQGTLVTVSSGGGGSGGGGSGGGGSAEAVVTQEPSLTVS |
| | PGGTVTLTCGSSTGAVTTSNYANWVQQKPGQAFRGLIGGTNKRAPGTPARFSGS |
| | LIGGKAALTITGAQPEDEAEYYCALWYSNLWVFGGGTKLTVLEPKSCDKTHTCP |
| | PCPAPEAAGAPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTI |
| | SKAKGQPREPQVYTLPPSRDELTKNQVSLWCLVKGFYPSDIAVEWESNGQPENN |
| | YKTTPPVLDSDGSFFLYSKLTVDKSRWQQGNVFSCSVMHEALHNHYTQKSLSLS |
| | PG (SEQ ID NO: 211) |
| 160C9_T4g | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDGGSGGSGGSGEVKLVESGGGLVQPGGSLKLSCAASGFTFNTYAMNWVR |
| | QAPGKGLEWVARIRSKYNNYATYYADSVKDRFTISRDDAKNTLYLQMNNLRTE |
| | DTAVYYCVRHGNFGNSYVSWFAYWGQGTLVTVSSGGGGSGGGGSGGGGSAQA |
| | VVTQEPSLTVSPGGTVTLTCRSSTGAVTTSNYANWVQQKPGQAPRGLIGGTNKR |
| | APGTPARFSGSLLGGKAALTLSGAQPEDEAEYYCALWYSNLWVFGGGTKLTVLE |
| | PKSCDKTHTCPPCPAPEAAGAPSVFLFPPKPKDTLMISRTPEVTCVVVDVSHEDPE |
| | VKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLWCLVKGFYPSDIAVE |
| | WESNGQPENNYKTTPPVLDSDGSFFLYSKLTVDKSRWQQGNVFSCSVMHEALHN |
| | HYTQKSLSLSPG (SEQ ID NO: 212) |
| 1B4_T4g | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDGGSGGSGGSGEVKLVESGGGLVQPGGSLKLSCAASGFTFNTYAMNWVR |
| | QAPGKGLEWVGRIRSKYNNYATYYADSVKDRFTISRDDSQNILYLQMNNLRTED |
| | TAVYYCVRHGNFGNSYVSWFAYWGQGTLVTVSSGGGGSGGGGSGGGGSAQAF |
| | VTQEPSLTVSPGGTVTLTCGSSTGAVTTSNYANWVQQKPGQAPRGLIGGTNKRA |
| | PGVPARFSGSLIGDKAALTITGAQPEDEAEYYCALWYSNLWVFGGGTKLTVLEPK |
| | SCDKTHTCPPCPAPEAAGAPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLWCLVKGFYPSDIAVEWE |
| | SNGQPENNYKTTPPVLDSDGSFFLYSKLTVDKSRWQQGNVFSCSVMHEALHNHY |
| | TQKSLSLSPG (SEQ ID NO: 213) |
| 4G2_T4g | QVQLVQSGAEVKKPGASVKVSCKASGYKFTSYWIEWVRQAPGQGLEWIGEILPG |
| | SDTTNYNEKFKDRVTFTSDTSTSTVYMELSSLRSEDTAVYYCARDRGNYRAWFG |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDGGSGGSGGSGEVKLLESGGGLVQPGGSLKLSCAASGFTFNTYAMNWVR |
| | QAPGKGLEWVGRIRSKYNNYATYYADSVKGRFTISRDDAKSILYLQMNNLKTED |
| | TAVYYCVRHGNFGNSYVSWFAYWGQGTLVTVSSGGGGSGGGGSGGGGSAEAV |
| | VTQEPSLTVSPGGTVTLTCGSSTGAVTTSNYANWVQQKPGQAFRGLIGGTNKRA |
| | PGTPARFSGSLIGGKAALTITGAQPEDEAEYYCALWYSNLWVFGGGTKLTVLEPK |
| | SCDKTHTCPPCPAPEAAGAPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLWCLVKGFYPSDIAVEWE |
| | SNGQPENNYKTTPPVLDSDGSFFLYSKLTVDKSRWQQGNVFSCSVMHEALHNHY |
| | TQKSLSLSPG (SEQ ID NO: 214) |

TABLE 13
Full length heavy chain sequences,
Type 5 format

| Clone ID | Full length heavy chain |
|---|---|
| sequence | (Type 5 HC) |
| 160C9_T5h | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDK |
| | THTEVKLVESGGGLVQPGGSLKLSC |
| | AASGFTFNTYAMNWVRQAPGKGLEW |
| | VARIRSKYNNYATYYADSVKDRFTI |
| | SRDDAKNTLYLQMNNLRTEDTAVYY |
| | CVRHGNFGNSYVSWFAYWGQGTLVT |
| | VSSGGGGSGGGGSGGGGSAQAVVTQ |
| | EPSLTVSPGGTVTLTCRSSTGAVTT |
| | SNYANWVQQKPGQAPRGLIGGTNKR |
| | APGTPARFSGSLLGGKAALTLSGAQ |
| | PEDEAEYYCALWYSNLWVFGGGTKL |
| | TVLEPKSCDKTHTCPPCPAPEAAGA |
| | PSVFLFPPKPKDTLMISRTPEVTCV |
| | VVDVSHEDPEVKFNWYVDGVEVHNA |
| | KTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPG |
| | (SEQ ID NO: 215) |
| 160C9_T5ss | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDK |
| | THTEVKLVESGGGLVQPGGSLKLSC |
| | AASGFTFNTYAMNWVRQAPGKGLEW |
| | VARIRSKYNNYATYYADSVKDRFTI |
| | SRDDAKNTLYLQMNNLRTEDTAVYY |
| | CVRHGNFGNSYVSWFAYWGQGTLVT |
| | VSSGGGGSGGGGSGGGGSAQAVVTQ |
| | EPSLTVSPGGTVTLTCRSSTGAVTT |
| | SNYANWVQQKPGQAPRGLIGGTNKR |
| | APGTPARFSGSLLGGKAALTLSGAQ |
| | PEDEAEYYCALWYSNLWVFGGGTKL |
| | TVLEPKSSDKTHTCPPCPAPEAAGA |
| | PSVFLFPPKPKDTLMISRTPEVTCV |
| | VVDVSHEDPEVKFNWYVDGVEVHNA |
| | KTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPG |
| | (SEQ ID NO: 239) |
| 1B4_T5h | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDK |
| | THTEVKLVESGGGLVQPGGSLKLSC |
| | AASGFTFNTYAMNWVRQAPGKGLEW |
| | VGRIRSKYNNYATYYADSVKDRFTI |
| | SRDDSQNILYLQMNNLRTEDTAVYY |
| | CVRHGNFGNSYVSWFAYWGQGTLVT |
| | VSSGGGGSGGGGSGGGGSAQAFVTQ |
| | EPSLTVSPGGTVTLTCGSSTGAVTT |
| | SNYANWVQQKPGQAPRGLIGGTNKR |
| | APGVPARFSGSLIGDKAALTITGAQ |
| | PEDEAEYYCALWYSNLWVFGGGTKL |
| | TVLEPKSCDKTHTCPPCPAPEAAGA |
| | PSVFLFPPKPKDTLMISRTPEVTCV |
| | VVDVSHEDPEVKFNWYVDGVEVHNA |
| | KTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPG |
| | (SEQ ID NO: 216) |
| 1B4_T5ss | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDK |
| | THTEVKLVESGGGLVQPGGSLKLSC |
| | AASGFTFNTYAMNWVRQAPGKGLEW |
| | VGRIRSKYNNYATYYADSVKDRFTI |
| | SRDDSQNILYLQMNNLRTEDTAVYY |
| | CVRHGNFGNSYVSWFAYWGQGTLVT |
| | VSSGGGGSGGGGSGGGGSAQAFVTQ |
| | EPSLTVSPGGTVTLTCGSSTGAVTT |
| | SNYANWVQQKPGQAPRGLIGGTNKR |
| | APGVPARFSGSLIGDKAALTITGAQ |
| | PEDEAEYYCALWYSNLWVFGGGTKL |
| | TVLEPKSSDKTHTCPPCPAPEAAGA |
| | PSVFLFPPKPKDTLMISRTPEVTCV |
| | VVDVSHEDPEVKFNWYVDGVEVHNA |
| | KTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPG |
| | (SEQ ID NO: 240) |
| 1B10_T5ss | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDK |
| | THTEVKLLESGGGLVQPGGSLKLSC |
| | AASGFTFNTYAMNWVRQAPGKGLEW |
| | VGRIRSKANNYATYYADSVKGRFTI |
| | SRDDSKNTLYLQMNNLKAEDTAVYY |
| | CVRHGNFGNSYVSWFAYWGQGTLVT |
| | VSSGGGGSGGGGSGGGGSAEAVVTQ |
| | EPSLTVSPGGTVTLTCGSSTGAVTT |
| | SNYANWVQQKPGQAPRGLIGGTNKR |
| | APGTPARFSGSLLGDKAALTITGAQ |
| | PEDEAEYYCALWYSNLWVFGGGTKL |
| | TVLEPKSSDKTHTCPPCPAPEAAGA |
| | PSVFLFPPKPKDTLMISRTPEVTCV |
| | VVDVSHEDPEVKFNWYVDGVEVHNA |
| | KTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPG |
| | (SEQ ID NO: 238) |
| 4G2_T5h | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDK |
| | THTEVKLLESGGGLVQPGGSLKLSC |
| | AASGFTFNTYAMNWVRQAPGKGLEW |
| | VGRIRSKYNNYATYYADSVKGRFTI |
| | SRDDAKSILYLQMNNLKTEDTAVYY |
| | CVRHGNFGNSYVSWFAYWGQGTLVT |
| | VSSGGGGSGGGGSGGGGSAEAVVTQ |
| | EPSLTVSPGGTVTLTCGSSTGAVTT |
| | SNYANWVQQKPGQAFRGLIGGTNKR |
| | APGTPARFSGSLIGGKAALTITGAQ |
| | PEDEAEYYCALWYSNLWVFGGGTKL |
| | TVLEPKSCDKTHTCPPCPAPEAAGA |
| | PSVFLFPPKPKDTLMISRTPEVTCV |
| | VVDVSHEDPEVKFNWYVDGVEVHNA |
| | KTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPG |
| | (SEQ ID NO: 217) |
| 4D4_T5h | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDK |
| | THTEVKLVESGGGLVQPGGSLKLSC |
| | AASGFTFNTYAMNWVRQAPGKGLEW |
| | VGRIRSKYNNYATYYADSVKGRFTI |
| | SRDDSKNTLYLQMNNLKTEDTAVYY |
| | CVRHGNFGNSYVSWFAYWGQGTLVT |
| | VSSGGGGSGGGGSGGGGSAQAVVTQ |
| | EPSLTVSPGGTVTLTCGSSTGAVTT |
| | SNYANWVQQKPGQAPRGLIGGTNKR |
| | APWTPARFSGSLIGDKAALTIAGAQ |
| | PEDEAEYYCALWYSNLWVFGGGTKL |
| | TVLEPKSCDKTHTCPPCPAPEAAGA |
| | PSVFLFPPKPKDTLMISRTPEVTCV |
| | VVDVSHEDPEVKFNWYVDGVEVHNA |
| | KTKPREEQYNSTYRVVSVLTVLHQD |
| | WLNGKEYKCKVSNKALPAPIEKTIS |
| | KAKGQPREPQVYTLPPSRDELTKNQ |
| | VSLTCLVKGFYPSDIAVEWESNGQP |
| | ENNYKTTPPVLDSDGSFFLYSKLTV |
| | DKSRWQQGNVFSCSVMHEALHNHYT |
| | QKSLSLSPG |
| | (SEQ ID NO: 218) |
| 160C9_T5d | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDG |
| | GGGSGGGGSEVKLVESGGGLVQPGG |
| | SLKLSCAASGFTFNTYAMNWVRQAP |
| | GKGLEWVARIRSKYNNYATYYADSV |
| | KDRFTISRDDAKNTLYLQMNNLRTE |
| | DTAVYYCVRHGNFGNSYVSWFAYWG |
| | QGTLVTVSSGGGGSGGGGSGGGGSA |
| | QAVVTQEPSLTVSPGGTVTLTCRSS |
| | TGAVTTSNYANWVQQKPGQAPRGLI |
| | GGTNKRAPGTPARFSGSLLGGKAAL |
| | TLSGAQPEDEAEYYCALWYSNLWVF |
| | GGGTKLTVLEPKSCDKTHTCPPCPA |
| | PEAAGAPSVFLFPPKPKDTLMISRT |
| | PEVTCVVVDVSHEDPEVKFNWYVDG |
| | VEVHNAKTKPREEQYNSTYRVVSVL |
| | TVLHQDWLNGKEYKCKVSNKALPAP |
| | IEKTISKAKGQPREPQVYTLPPSRD |
| | ELTKNQVSLTCLVKGFYPSDIAVEW |
| | ESNGQPENNYKTTPPVLDSDGSFFL |
| | YSKLTVDKSRWQQGNVFSCSVMHEA |
| | LHNHYTQKSLSLSPG |
| | (SEQ ID NO: 219) |
| 1B4_T5d | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDG |
| | GGGSGGGGSEVKLVESGGGLVQPGG |
| | SLKLSCAASGFTFNTYAMNWVRQAP |
| | GKGLEWVGRIRSKYNNYATYYADSV |
| | KDRFTISRDDSQNILYLQMNNLRTE |
| | DTAVYYCVRHGNFGNSYVSWFAYWG |
| | QGTLVTVSSGGGGSGGGGSGGGGSA |
| | QAFVTQEPSLTVSPGGTVTLTCGSS |
| | TGAVTTSNYANWVQQKPGQAPRGLI |
| | GGTNKRAPGVPARFSGSLIGDKAAL |
| | TITGAQPEDEAEYYCALWYSNLWVF |
| | GGGTKLTVLEPKSCDKTHTCPPCPA |
| | PEAAGAPSVFLFPPKPKDTLMISRT |
| | PEVTCVVVDVSHEDPEVKFNWYVDG |
| | VEVHNAKTKPREEQYNSTYRVVSVL |
| | TVLHQDWLNGKEYKCKVSNKALPAP |
| | IEKTISKAKGQPREPQVYTLPPSRD |
| | ELTKNQVSLTCLVKGFYPSDIAVEW |
| | ESNGQPENNYKTTPPVLDSDGSFFL |
| | YSKLTVDKSRWQQGNVFSCSVMHEA |
| | LHNHYTQKSLSLSPG |
| | (SEQ ID NO: 220) |
| 160C9_T5g | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDG |
| | GSGGSGGSGEVKLVESGGGLVQPGG |
| | SLKLSCAASGFTFNTYAMNWVRQAP |
| | GKGLEWVARIRSKYNNYATYYADSV |
| | KDRFTISRDDAKNTLYLQMNNLRTE |
| | DTAVYYCVRHGNFGNSYVSWFAYWG |
| | QGTLVTVSSGGGGSGGGGSGGGGSA |
| | QAVVTQEPSLTVSPGGTVTLTCRSS |
| | TGAVTTSNYANWVQQKPGQAPRGLI |
| | GGTNKRAPGTPARFSGSLLGGKAAL |
| | TLSGAQPEDEAEYYCALWYSNLWVF |
| | GGGTKLTVLEPKSCDKTHTCPPCPA |
| | PEAAGAPSVFLFPPKPKDTLMISRT |
| | PEVTCVVVDVSHEDPEVKFNWYVDG |
| | VEVHNAKTKPREEQYNSTYRVVSVL |
| | TVLHQDWLNGKEYKCKVSNKALPAP |
| | IEKTISKAKGQPREPQVYTLPPSRD |
| | ELTKNQVSLTCLVKGFYPSDIAVEW |
| | ESNGQPENNYKTTPPVLDSDGSFFL |
| | YSKLTVDKSRWQQGNVFSCSVMHEA |
| | LHNHYTQKSLSLSPG |
| | (SEQ ID NO: 221) |
| 1B4_T5g | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDG |
| | GSGGSGGSGEVKLVESGGGLVQPGG |
| | SLKLSCAASGFTFNTYAMNWVRQAP |
| | GKGLEWVGRIRSKYNNYATYYADSV |
| | KDRFTISRDDSQNILYLQMNNLRTE |
| | DTAVYYCVRHGNFGNSYVSWFAYWG |
| | QGTLVTVSSGGGGSGGGGSGGGGSA |
| | QAFVTQEPSLTVSPGGTVTLTCGSS |
| | TGAVTTSNYANWVQQKPGQAPRGLI |
| | GGTNKRAPGVPARFSGSLIGDKAAL |
| | TITGAQPEDEAEYYCALWYSNLWVF |
| | GGGTKLTVLEPKSCDKTHTCPPCPA |
| | PEAAGAPSVFLFPPKPKDTLMISRT |
| | PEVTCVVVDVSHEDPEVKFNWYVDG |
| | VEVHNAKTKPREEQYNSTYRVVSVL |
| | TVLHQDWLNGKEYKCKVSNKALPAP |
| | IEKTISKAKGQPREPQVYTLPPSRD |
| | ELTKNQVSLTCLVKGFYPSDIAVEW |
| | ESNGQPENNYKTTPPVLDSDGSFFL |
| | YSKLTVDKSRWQQGNVFSCSVMHEA |
| | LHNHYTQKSLSLSPG |
| | (SEQ ID NO: 222) |
| 4G2_T5g | QVQLVQSGAEVKKPGASVKVSCKAS |
| | GYKFTSYWIEWVRQAPGQGLEWIGE |
| | ILPGSDTTNYNEKFKDRVTFTSDTS |
| | TSTVYMELSSLRSEDTAVYYCARDR |
| | GNYRAWFGYWGQGTLVTVSSASTKG |
| | PSVFPLAPSSKSTSGGTAALGCLVK |
| | DYFPEPVTVSWNSGALTSGVHTFPA |
| | VLQSSGLYSLSSVVTVPSSSLGTQT |
| | YICNVNHKPSNTKVDKKVEPKSCDG |
| | GSGGSGGSGEVKLLESGGGLVQPGG |
| | SLKLSCAASGFTFNTYAMNWVRQAP |
| | GKGLEWVGRIRSKYNNYATYYADSV |
| | KGRFTISRDDAKSILYLQMNNLKTE |
| | DTAVYYCVRHGNFGNSYVSWFAYWG |
| | QGTLVTVSSGGGGSGGGGSGGGGSA |
| | EAVVTQEPSLTVSPGGTVTLTCGSS |
| | TGAVTTSNYANWVQQKPGQAFRGLI |
| | GGTNKRAPGTPARFSGSLIGGKAAL |
| | TITGAQPEDEAEYYCALWYSNLWVF |
| | GGGTKLTVLEPKSCDKTHTCPPCPA |
| | PEAAGAPSVFLFPPKPKDTLMISRT |
| | PEVTCVVVDVSHEDPEVKFNWYVDG |
| | VEVHNAKTKPREEQYNSTYRVVSVL |
| | TVLHQDWLNGKEYKCKVSNKALPAP |
| | IEKTISKAKGQPREPQVYTLPPSRD |
| | ELTKNQVSLTCLVKGFYPSDIAVEW |
| | ESNGQPENNYKTTPPVLDSDGSFFL |
| | YSKLTVDKSRWQQGNVFSCSVMHEA |
| | LHNHYTQKSLSLSPG |
| | (SEQ ID NO: 223) |

TABLE 14
Full length heavy chain sequences, Type 1 format

| | Full length heavy chain |
|---|---|
| Clone ID | sequence (Type 1 HC) |
| 4G2_T1 | EVKLLESGGGLVQPGGSLKLSCAASGFTFN |
| | TYAMNWVRQAPGKGLEWVGRIRSKYNNYAT |
| | YYADSVKGRFTISRDDAKSILYLQMNNLKT |
| | EDTAVYYCVRHGNFGNSYVSWFAYWGQGTL |
| | VTVSSGGGGSGGGGSGGGGSAEAVVTQEPS |
| | LTVSPGGTVTLTCGSSTGAVTTSNYANWVQ |
| | QKPGQAFRGLIGGTNKRAPGTPARFSGSLI |
| | GGKAALTITGAQPEDEAEYYCALWYSNLWV |
| | FGGGTKLTVLEPKSCDKTHTCPPCPAPEAA |
| | GAPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPAPIEKTISKAKGQPREPQVYTLPPSR |
| | DELTKNQVSLWCLVKGFYPSDIAVEWESNG |
| | QPENNYKTTPPVLDSDGSFFLYSKLTVDKS |
| | RWQQGNVFSCSVMHEALHNHYTQKSLSLSP |
| | G (SEQID NO: 224) |
| 160C9_T1 | EVKLVESGGGLVQPGGSLKLSCAASGFTFN |
| | TYAMNWVRQAPGKGLEWVARIRSKYNNYAT |
| | YYADSVKDRFTISRDDAKNTLYLQMNNLRT |
| | EDTAVYYCVRHGNFGNSYVSWFAYWGQGTL |
| | VTVSSGGGGSGGGGSGGGGSAQAVVTQEPS |
| | LTVSPGGTVTLTCRSSTGAVTTSNYANWVQ |
| | QKPGQAPRGLIGGTNKRAPGTPARFSGSLL |
| | GGKAALTLSGAQPEDEAEYYCALWYSNLWV |
| | FGGGTKLTVLEPKSCDKTHTCPPCPAPEAA |
| | GAPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPAPIEKTISKAKGQPREPQVYTLPPSR |
| | DELTKNQVSLWCLVKGFYPSDIAVEWESNG |
| | QPENNYKTTPPVLDSDGSFFLYSKLTVDKS |
| | RWQQGNVFSCSVMHEALHNHYTQKSLSLSP |
| | G |
| | (SEQ ID NO: 225) |
| 1B4_T1 | EVKLVESGGGLVQPGGSLKLSCAASGFTFN |
| | TYAMNWVRQAPGKGLEWVGRIRSKYNNYAT |
| | YYADSVKDRFTISRDDSQNILYLQMNNLRT |
| | EDTAVYYCVRHGNFGNSYVSWFAYWGQGTL |
| | VTVSSGGGGSGGGGSGGGGSAQAFVTQEPS |
| | LTVSPGGTVTLTCGSSTGAVTTSNYANWVQ |
| | QKPGQAPRGLIGGTNKRAPGVPARFSGSLI |
| | GDKAALTITGAQPEDEAEYYCALWYSNLWV |
| | FGGGTKLTVLEPKSCDKTHTCPPCPAPEAA |
| | GAPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPAPIEKTISKAKGQPREPQVYTLPPSR |
| | DELTKNQVSLWCLVKGFYPSDIAVEWESNG |
| | QPENNYKTTPPVLDSDGSFFLYSKLTVDKS |
| | RWQQGNVFSCSVMHEALHNHYTQKSLSLSP |
| | G (SEQ |
| | ID NO: 226) |
| 4D4_T1 | EVKLVESGGGLVQPGGSLKLSCAASGFTFN |
| | TYAMNWVRQAPGKGLEWVGRIRSKYNNYAT |
| | YYADSVKGRFTISRDDSKNTLYLQMNNLKT |
| | EDTAVYYCVRHGNFGNSYVSWFAYWGQGTL |
| | VTVSSGGGGSGGGGSGGGGSAQAVVTQEPS |
| | LTVSPGGTVTLTCGSSTGAVTTSNYANWVQ |
| | QKPGQAPRGLIGGTNKRAPWTPARFSGSLI |
| | GDKAALTIAGAQPEDEAEYYCALWYSNLWV |
| | FGGGTKLTVLEPKSCDKTHTCPPCPAPEAA |
| | GAPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPAPIEKTISKAKGQPREPQVYTLPPSR |
| | DELTKNQVSLWCLVKGFYPSDIAVEWESNG |
| | QPENNYKTTPPVLDSDGSFFLYSKLTVDKS |
| | RWQQGNVFSCSVMHEALHNHYTQKSLSLSP |
| | G |
| | (SEQ ID NO: 227) |
| 161H2_T1 | EVKLVESGGGLVQPGGSLKLSCAASGFTFN |
| | TYAMNWVRQAPGKGLEWVARIRSKYNNYAT |
| | YYADSVKDRFTISRDDAKNILYLQMNNLRT |
| | EDTAVYYCVRHGNFGNSYVSWFAYWGQGTL |
| | VTVSSGGGGSGGGGSGGGGSAQAVVTQEPS |
| | LTVSPGGTVTLTCGSSTGAVTTSNYANWVQ |
| | QKPGQAPRGLIGGTNKRAPGVPARFSGSLL |
| | GGKAALTLSGAQPEDEAEYYCALWYSNLWV |
| | FGGGTKLTVLEPKSCDKTHTCPPCPAPEAA |
| | GAPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPAPIEKTISKAKGQPREPQVYTLPPSR |
| | DELTKNQVSLWCLVKGFYPSDIAVEWESNG |
| | QPENNYKTTPPVLDSDGSFFLYSKLTVDKS |
| | RWQQGNVFSCSVMHEALHNHYTQKSLSLSP |
| | G |
| | (SEQ ID NO: 228) |
| 1B10_T1 | EVKLLESGGGLVQPGGSLKLSCAASGFTFN |
| | TYAMNWVRQAPGKGLEWVGRIRSKANNYAT |
| | YYADSVKGRFTISRDDSKNTLYLQMNNLKA |
| | EDTAVYYCVRHGNFGNSYVSWFAYWGQGTL |
| | VTVSSGGGGSGGGGSGGGGSAEAVVTQEPS |
| | LTVSPGGTVTLTCGSSTGAVTTSNYANWVQ |
| | QKPGQAPRGLIGGTNKRAPGTPARFSGSLL |
| | GDKAALTITGAQPEDEAEYYCALWYSNLWV |
| | FGGGTKLTVLEPKSSDKTHTCPPCPAPEAA |
| | GAPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPAPIEKTISKAKGQPREPQVYTLPPSR |
| | DELTKNQVSLWCLVKGFYPSDIAVEWESNG |
| | QPENNYKTTPPVLDSDGSFFLYSKLTVDKS |
| | RWQQGNVFSCSVMHEALHNHYTQKSLSLSP |
| | G |
| | (SEQ ID NO: 232) |

### Preparation of Binding Compounds

The multispecific binding compounds of the present invention can be prepared by methods known in the art. For example, binding compounds and antigen-binding fragments thereof can also be produced by recombinant DNA technology, by expression of the encoding nucleic acid in a suitable eukaryotic or prokaryotic host, including, for example, mammalian cells (e.g., CHO cells), E. coli or yeast.

### Pharmaceutical Compositions, Uses and Methods of Treatment

It is another aspect of the present invention to provide pharmaceutical compositions comprising one or more multispecific binding compounds of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

In one embodiment, a pharmaceutical composition comprises a multispecific binding compound that binds to LRRC15. In another embodiment, a pharmaceutical composition comprises a multispecific binding compound with binding specificity for two or more non-overlapping epitopes on an LRRC15 protein. In a preferred embodiment, a pharmaceutical composition comprises a multispecific binding compound with binding specificity to LRRC15 and with binding specificity to a binding target on an effector cell (e.g., a binding target on a T cell, such as, e.g., a CD3 protein on a T cell).

Pharmaceutical compositions of the binding compounds used in accordance with the present invention are prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The binding compounds herein can be administered by intravenous injection or infusion or subcutaneously. For injection administration, the binding compounds herein can be formulated in aqueous solutions, preferably in physiologically-compatible buffers to reduce discomfort at the site of injection. The solution can contain carriers, excipients, or stabilizers as discussed above. Alternatively, binding compounds can be in lyophilized form for constitution with a suitable vehicle, e.g., sterile pyrogen-free water, before use.

Antibody formulations are disclosed, for example, in U.S. Pat. No. 9,034,324 Similar formulations can be used for the binding compounds of the present invention. Subcutaneous antibody formulations are described, for example, in US20160355591 and US20160166689.

### Methods of Use

The multispecific binding compounds and pharmaceutical compositions described herein can be used for the treatment of diseases and conditions characterized by the expression of LRRC15, including, without limitation, the conditions and diseases described above.

LRRC15 has been identified as being highly expressed in multiple solid tumor indications, including on some tumor-associated stroma, with limited expression in normal tissue. Purcell et al., Cancer Res; 78(14); 4059-72. Due to its limited expression in normal tissue, LRRC15 is an attractive target for the treatment of malignancies that are characterized by the expression of LRRC15.

In one aspect, the multispecific binding compounds and pharmaceutical compositions herein can be used to treat cancers that are characterized by expression of LRRC15. As used herein, a cancer that is “characterized by expression of LRRC15” includes, without limitation, a cancer wherein one or more tumor cells express LRRC15, and/or wherein tumor-associated stroma exhibits expression of LRRC15. Such cancers include, without limitation, hematological malignancies that are characterized by the expression of LRRC15, including, without limitation, large B-cell lymphoma. In another aspect, the multispecific binding compounds and pharmaceutical compositions herein can be used to treat solid tumors that are characterized by expression of LRRC15, including, without limitation, breast, lung, pancreatic, and ovarian cancers. In yet another aspect, the multispecific binding compounds and pharmaceutical compositions herein can be used to treat sarcomas that are characterized by expression of LRRC15.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration. Dosage unit forms generally contain between from about 1 mg to about 500 mg of an active ingredient.

In some embodiments, the therapeutic dosage the agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Typically, compositions are prepared as injectables, either as liquid solutions or suspensions; solid forms suitable for solution in, or suspension in, liquid vehicles prior to injection can also be prepared. The pharmaceutical compositions herein are suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the antibodies and antibody structures described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in humans The dosage of the antibodies described herein lies preferably within a range of circulating concentrations that include the effective dose with little or no toxicity. The dosage can vary within this range depending upon the dosage form employed and the route of administration utilized The exact formulation, route of administration and dosage can be chosen by the individual physician in view of the patient's condition.

The compositions for administration will commonly comprise an antibody or other agent (e.g., another ablative agent) dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents and formulations thereof, of the invention and instructions for use. The kit can further contain a least one additional reagent, e.g., a chemotherapeutic drug, etc. Kits typically include a label indicating the intended use of the contents of the kit. The term “label” as used herein includes any writing, or recorded material supplied on or with a kit, or which otherwise accompanies a kit.

The invention now being fully described, it will be apparent to one of ordinary skill in the art that various changes and modifications can be made without departing from the spirit or scope of the invention.

### EXAMPLES

### Example 1: Library Construction and Phage Display

A humanization/optimization library was constructed based on the following principals. Mouse CDRs would be unchanged to maintain binding to human and cynomolgus CD3ε Human frameworks were analyzed for homology to the mouse framework. Diversity of the framework differences were based on the mouse and human framework amino acids. In addition, 890 framework-family antibodies were analyzed to preserve co-variant amino acid diversity with the hypothesis that amino acids that were maintained throughout evolution of the framework could lead to improved stability. Combinatorial diversity of the VH was 1.26E7 and VL diversity was 2.56E2.

This diversity was encoded in Ultramers (IDT) which included designed amino acids at each position. Construction of the V regions was facilitated by overlapping conserved regions in splicing PCR reactions. The full length VL and VH segments were rescued by end primers using high fidelity PCR. ScFv library insert pool was completed by PCR of the VH and VL pools with the (G4S)3 linker (SEQ ID NO: 229) in the format: VH-linker-VL-6his tag-myc tag-amber stop_g3p. The full library fragment pool was digested with SfiI restriction enzyme and cloned using an Electroligase (New England Biolabs) into the vector pADL23c (Antibody Design Labs) digested with Bgl1. Finally, NEB5 F′Iq electrocompetent cells were transformed with the cloned phagemids and the culture was grown to log phase. Phages expressing scFv clones were packaged with the helper M13K07 and cultured overnight. The purified phage library was heated to 65° C. for 10 minutes to reduce poorly folded clones in the library. Phage expressing complete scFv clones were captured by preparative scale Nanolink streptavidin magnetic beads coated with biotinylated goat anti-Myc antibody and propagated in a NEB F′Iq phage culture.

The resulting library was stored and used for plate-based phage display and bead-based phage display of Cynomolgus CD3ε 1-27aa-Fc. In addition, panning on Cynomolgus CD3ε 1-27aa-Fc followed by subsequent rounds of binding to human CD3ε 1-27aa-Fc was done to maintain cross-reactive clones. In some panning experiments, binding at room temperature was followed by incubation at 65° C. and stringent washes to enrich stable clones. Target bound phage were eluted by acidic glycine buffer and neutralized prior to plating colonies for titering and sequencing. HB2151 E. coli were infected with target-enriched eluted phage to express soluble scFv proteins which were evaluated in enzyme-linked immunosorbent assay ELISA screens.

### Example 2: Vector Construction

Vector pcDNA3.4TOPO (Invitrogen) was ligated to a short polylinker containing EcoRI, XhoI, and NotI. The resulting plasmid was digested with EcoRl and Notl restriction enzymes and purified by gel electrophoresis. For heavy chain cloning, the Gibson method was used to assemble the prepared vector with the appropriate DNA fragment encoding a VH region and the human IgG1 AAA fragment (CH1 to CH3 domains). Variable light regions were constructed with a similar method using gblocks to assemble Vkappa regions with a gblock fragment which encoded the constant kappa (Ck). ScFv-Fc expressing plasmids used gblock fragments (IDT) to encode the scFv and a PCR fragment to encode the antibody hinge to CH3 domain of IgG1. The symmetrical formats, scFv-Fc, Type 2, and Type 5 (FIG. 3, Panels A, C, and F), contained the IGHG1 Fc sequence with three mutations to disrupt Fcg receptor interactions and compliment binding: L234A, L235A, and G237A which yield the sequence (from the hinge region) EPKSCDKTHTCPPCPAPEAAGA (SEQ ID NO: 230). In preferred embodiments, a C220S mutation was included into the upper hinge region in formats where the scFv links to the hinge Fc (SEQ ID NO: 241 and 242) (see, e.g., US 2010/0233173A1; WO2018/071919 A1; US2016/0009824 A1; WO2014/144357; and EP 3511342A1, the disclosures of which are incorporated by reference herein in their entireties). Asymmetrical formats, such as Type 1 and Type 4 (FIG. 3, Panels B and E) shared the same Fc sequence with the addition of “knob” or “hole” mutations (see, e.g., Ridgway et al., Protein Eng. 1996 July;9(7):17-21; U.S. Pat. No. 8,216,805). In preferred embodiments, mutations S354C (knob Fc), Y349C (hole Fc) (Merchant et al., Nature Biotech. 1998 July;16:677-681) created a disulfide bond between the CH3 domains to aid production of the bispecific antibody. In further preferred embodiments, addition of protein A non-binding mutations H435R, Y436F (Jendeberg et al., J. Immuno. Methods 1997; 201:25-34) to the knob Fc facilitated purification of asymmetrical formats. Type 2 formats which presented the anti-CD3ε scFv at the C-terminus of the Fc domain were constructed by removing the terminal lysine from the Fc and cloning the scFv and a linker comprising G and S amino acid residues (Table 9 shows various linkers that were evaluated). Type 4 and Type 5 extended heavy chain fragments were cloned with the following structure: [anti-LRRC15 VH]-[CH1]-[linker]-[anti CD3 scFv]-[hinge-CH2-CH3], where linked contained the sequence EPKSCDKTHT (SEQ ID NO: 189) or EPKSSDKTHT (SEQ ID NO:241), EPKSCDGGSGGSGGSG (SEQ ID NO:190), or EPKSCDGGGGSGGGGS (SEQ ID NO:191). All assembly was done with the Gibson method (NEB).

### Example 3: Protein Expression

Plasmids were prepped and transfected into Expi293 or ExpiCHO cells using the transient expression system (Thermo Fisher). Briefly, plasmids were transfected into 3e6 cells/mL cells at 1 ug plasmid DNA total/mL culture. Heavy chain and light chain plasmids were mixed in a 1:1 ratio. Bispecific binding compound transfections used increased light chain plasmid relative to two separate heavy chain plasmids. Cultures were incubated at 37° C., shaking After 16 hours, Transfection Enhancer 1 and 2 was added to the cultures and incubation was continued for six days. Supernatants were filtered, and protein titers were determined by an IgG quantitation protocol using the Octet Red96 (Pall). IgG was purified by Mab Select Sure Protein-A column purification on an ACTA PURE system and dialyzed overnight in PBS. Asymmetric bispecific antibodies (Type 1 and Type 4, FIG. 3, Panels B and E) usually required additional purification, which typically involved preparative size exclusion chromatography (pSEC).

### Example 4: Protein Thermal Shift of Humanized anti-LRRC15 x CD3 Bispecific Binding Compounds

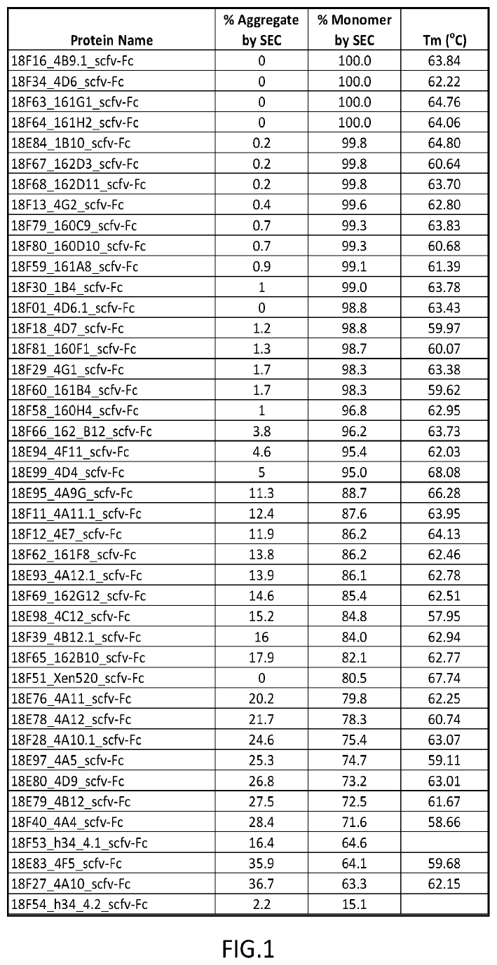

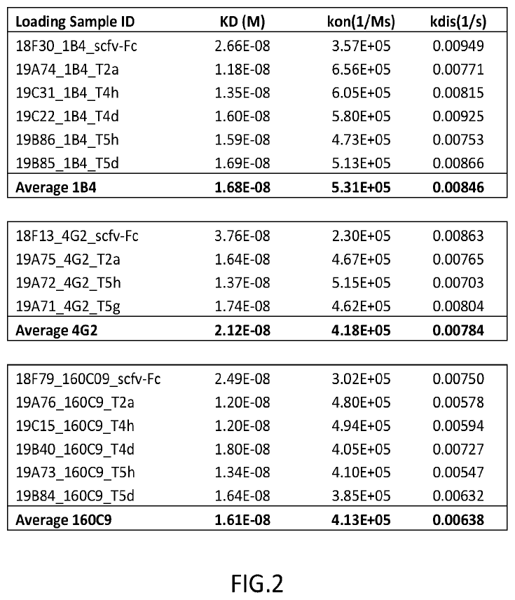

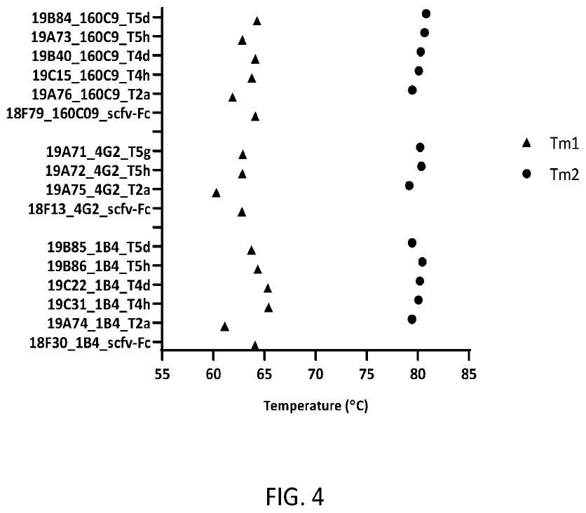

Ten (10) ug/mL anti-LRRC15 x CD3 bispecific binding compounds were mixed with 2 ul 50X protein thermal shift dye, and PBS to a final volume of 100 ul. Samples were aliquoted into a PCR 96-tube plate in quadruplicate (25 ul/well). Protein thermal shift reactions were measured on an Applied Biosystems StepOne Real-Time PCR instrument using a continuous temperature gradient of 1° C. change per 1 min 5 sec from 22-95° C. Tm was analyzed using the derivative method. The results show that the scFv Tm ranges from 60-66 ° C. when 3 clones, 160C9, 4G2, and 1B4, are presented in different formats with different linkers (FIG. 4). The Tm of anti-CD3 clone 160C9 scFv-Fc is approximately 64° C. (see FIGS. 1 and 4) and drops to approximately 62° C. when the scFv is presented at the C-terminus in the T2a format (Type 2 format with a GS linker). A drop in the Tm was observed in two additional clones as well (see FIG. 4). Generally, the Type 4 and Type 5 formats showed a similar or slightly improved Tm compared to the scFv-Fc format depicted in FIG. 3, Panel A.

### Example 5: Flow Cytometry Binding Analysis of Anti-CD3ε Antibodies to T Cells, Jurkat Cells, and H-SCF Cells

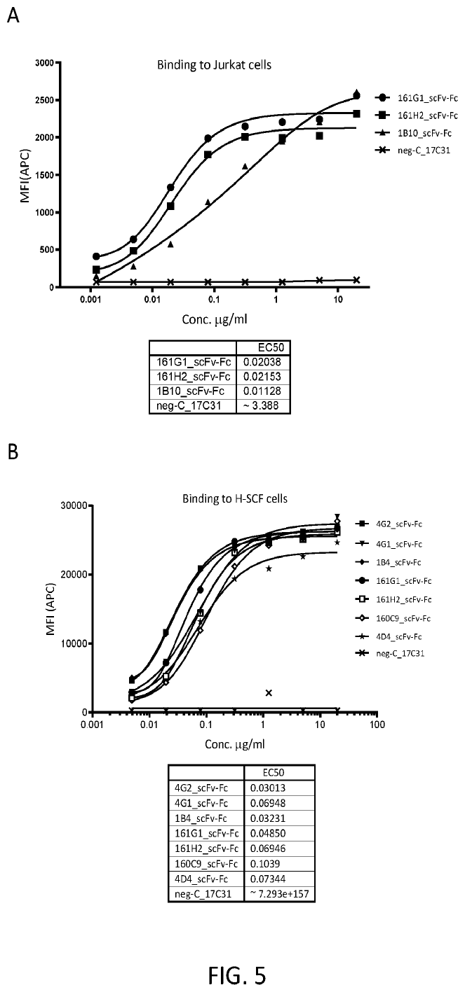

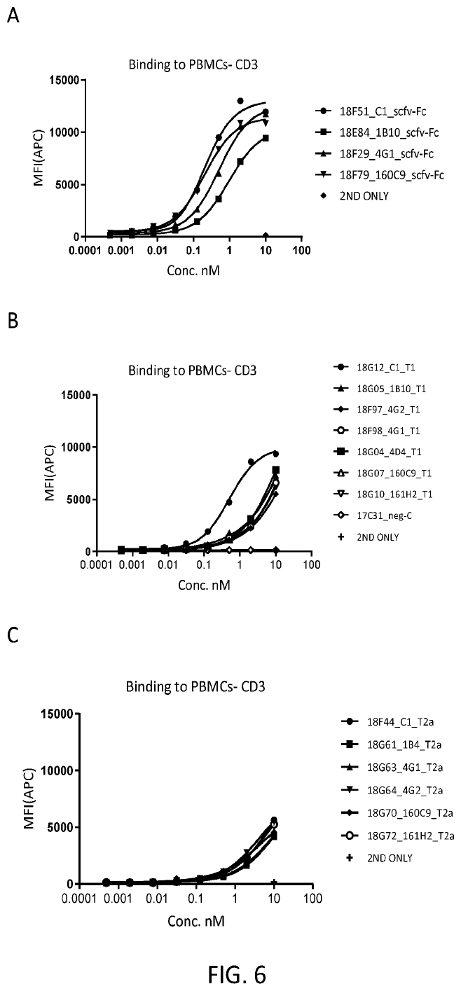

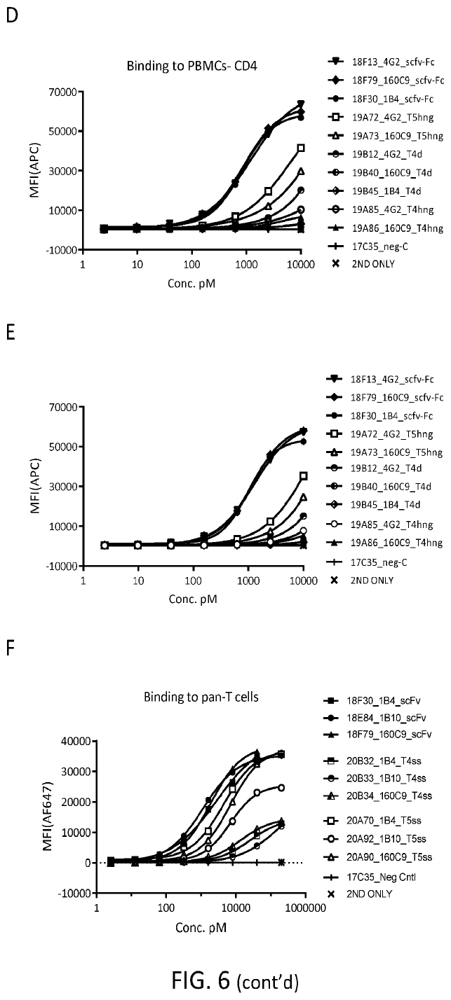

Peripheral blood mononuclear cells (PBMCs), Jurkat, or H-SCF (Cynomolgus T cell) cells were prepared by standard methods, washed with FACS buffer, and distributed into 96-well v-bottom polypropylene plates at 200,000 cells/well. ScFv-Fc proteins or positive control antibody (BD Biosciences 556610) were serially diluted from 40 ug/mL and used to stain the cells on ice for 20 minutes. Cells were washed in FACS buffer, stained with 1:500 secondary antibody goat anti-hu IgG Fc-AF647 (Goat anti-ms-IgG Fc-AF647 for control) for 25 minutes on ice, washed again and resuspended in buffer containing 7-AAD before analysis by flow cytometry. PBMCs were stained for CD3-FITC, CD4-APC-H7, CD8-PE expression in addition to the bispecific antibodies, which were detected by goat anti-hu IgG Fc-AF647 as described above. The results demonstrate several scFv-Fc clones robustly bind to human Jukat cells (FIG. 5A), cynomolgus T cell line H-SCF (FIG. 5B), and CD3+ cells in prepared human PBMCs (FIG. 6A). Binding to CD3+ PBMCs is reduced in Type 1 (FIG. 6B) and Type 2 (FIG. 6C). Similarly, anti-CD3 binding of Type 5 bispecifics to CD4+ T cells is reduced, and binding of Type 4 is further reduced, compared to scFv-Fc (FIG. 6D). FIG. 6E and FIG. 6F demonstrates a similar pattern of binding to CD8+ and pan T cells, respectively, in which binding signal is reduced in Type 5, and further reduced in Type 4 formats.

### Example 6: Flow Cytometry Binding Analysis of U118MG Cells

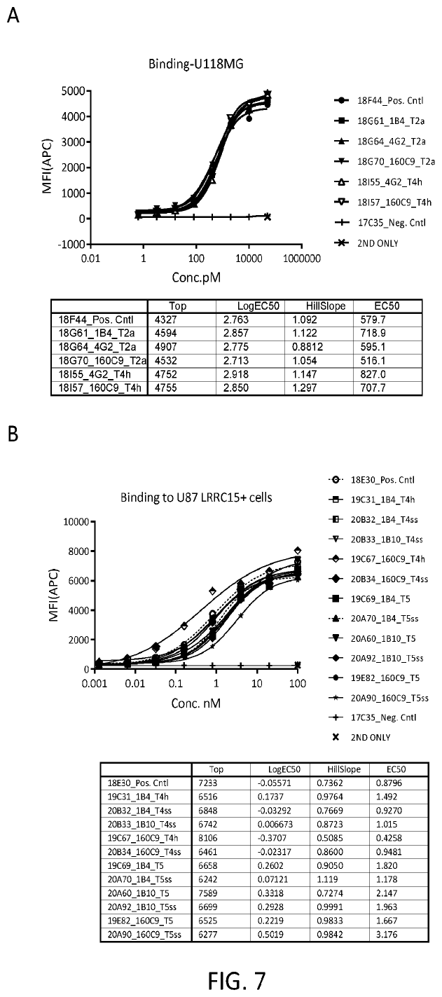

U118MG or U87MG cells were harvested by Trypsin, washed with FACS buffer, and resuspended at 5E6 cells/mL, and aliquoted into in 96 well plates at 1E5 cells/well. Cells were stained with serial dilutions of anti-LRRC15 binding compounds, bispecific binding compounds, positive control antibody “C1-IgG1”, or isotype IgG1 (starting concentration 50 nM) on ice for 45 minutes followed by washing and secondary staining with 1:500 diluted Goat anti-human IgG-AF647. Cells were analyzed by flow cytometry following a final wash and addition of 7-AAD. The results are shown in FIG. 7, and demonstrate robust binding to LRRC15 positive U118MG (FIG. 7A) and U87MG (FIG. 7B) cells.

### Example 7: Tumor Dependent T Cell Activation Assay

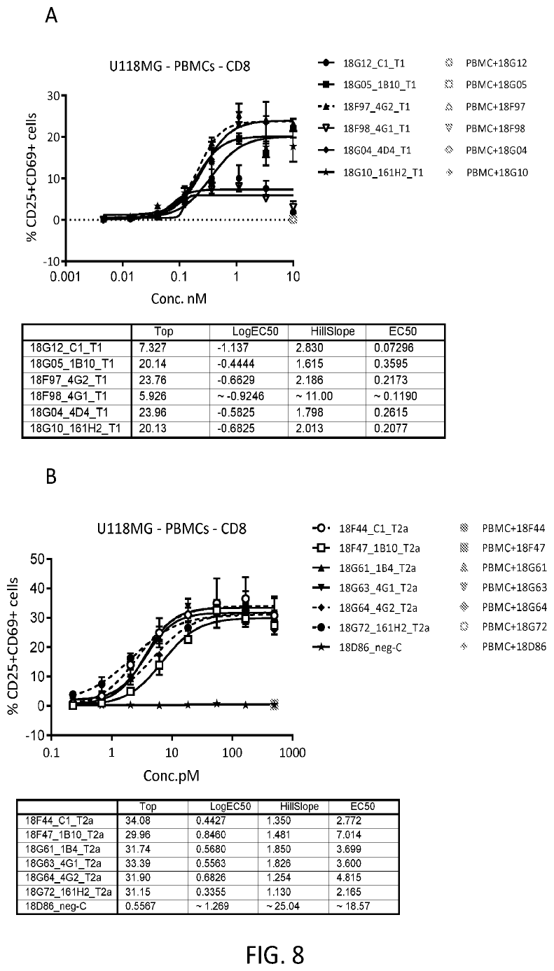

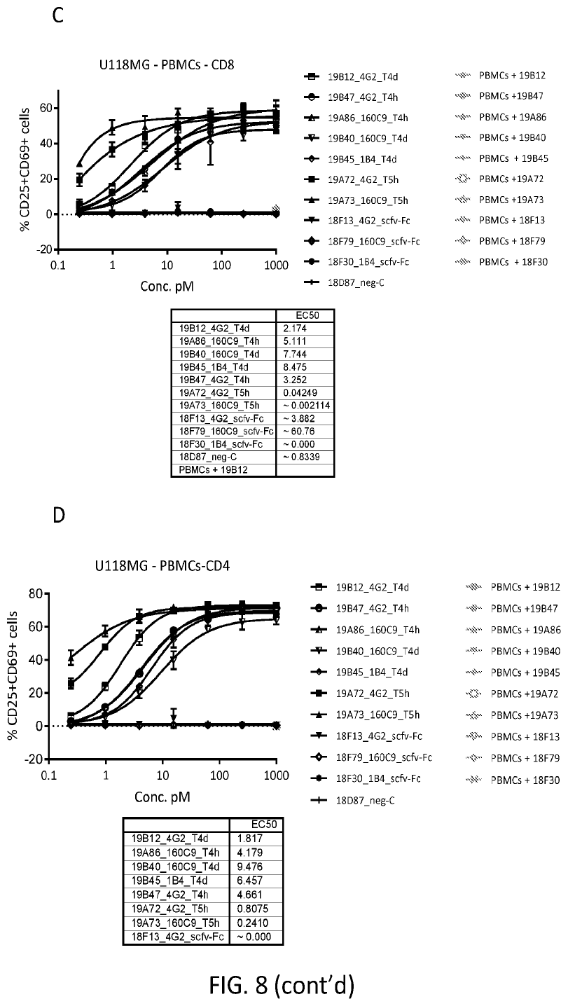

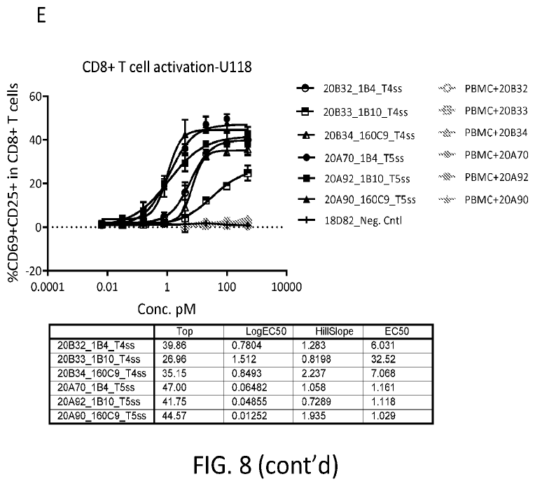

RPMI7951, U118MG, U87MG, or A431 (LRRC15 negative) tumor cells were prepared at 2E5 cells/mL in media containing RPMI 10% human serum, distributed into a flat-bottom 96-well plate at 1E4 cells/well, and incubated overnight. On the following day, fresh PBMCs were prepared by standard techniques at 4E6 cells/mL in RPMI+10% human serum. These cells and the diluted bispecific binding compounds, or control binding compounds, were then added to the wells containing the tumor cells at a 10:1 Effector:Target cell ratio, and the plates were incubated for 48 hours prior to flow cytometry analysis of the cells. Control wells lacked tumor cells to test for direct activation of T cells by the anti-CD3-containing bispecific binding compounds. Supernatants were frozen for cytokine detection. Cells were analyzed by flow cytometry with the following primary reagent antibodies: CD3-FITC, CD4-APC-H7, CD8-PE, CD25-BV421, CD69-APC, 7-AAD, Isotype-APC, Isotype-BV421, Isotype-APC diluted in FACS buffer. Following sample acquisition, data was analyzed using FlowJo by gating on (P1)FSC/SSC, (P2)FSC-AxFSC-H for single cells, followed by (P3) CD3x7AAD live/dead cell gating, then CD4(P4)xCD8(P5). Activation was assessed by CD25 and CD69 markers of CD3+/CD8+ T cells and CD3+/CD4+ T cells. The results demonstrate that LRRC15xCD3 Type 1 clones activate CD8+ T cells in the presence of U118MG tumor cells, but not in the absence of tumor cells (FIG. 8A). Similarly, LRRC15xCD3 Type 2 clones (FIG. 8B), Type 4 and Type 5 clones (FIG. 8C and FIG. 8E) activate CD8+ T cells in a U118MG tumor cell dependent manner.

### Exmaple 8: T Cell Proliferation Assay

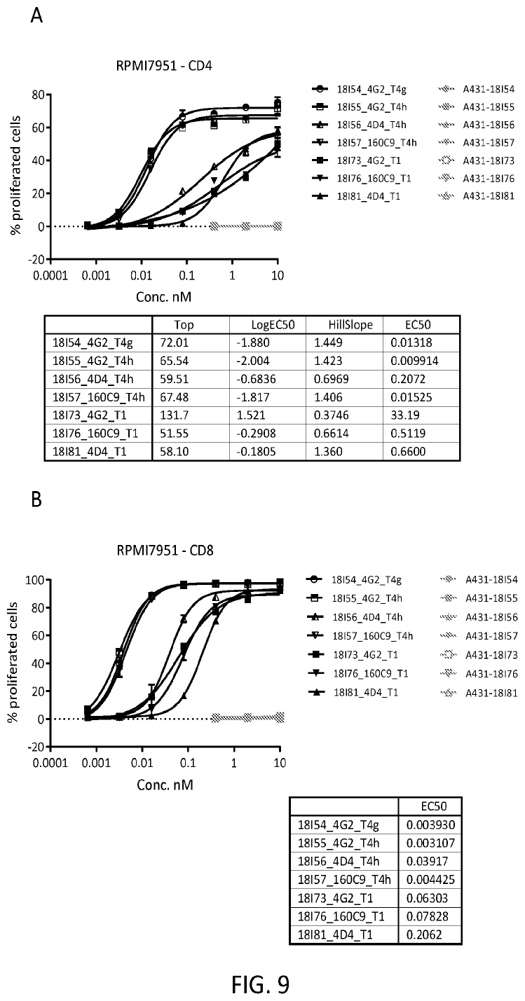

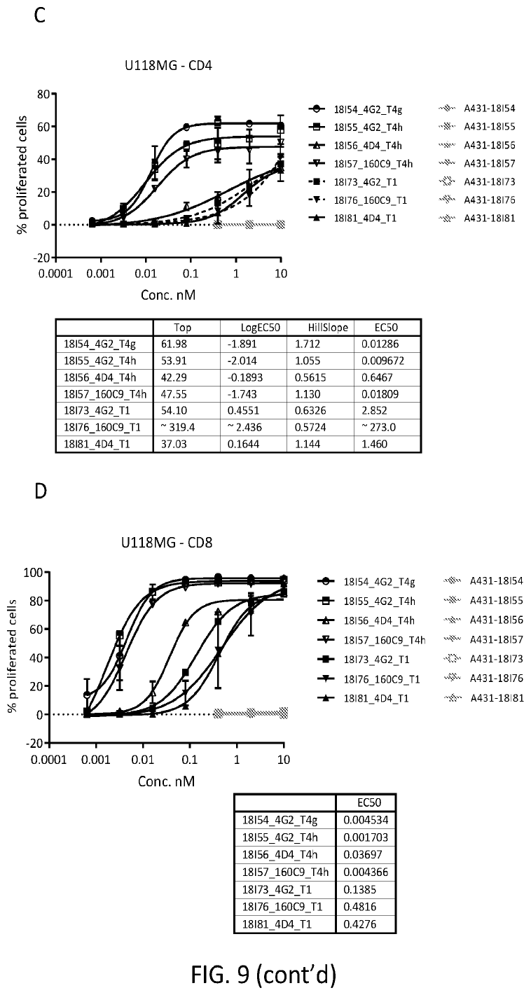

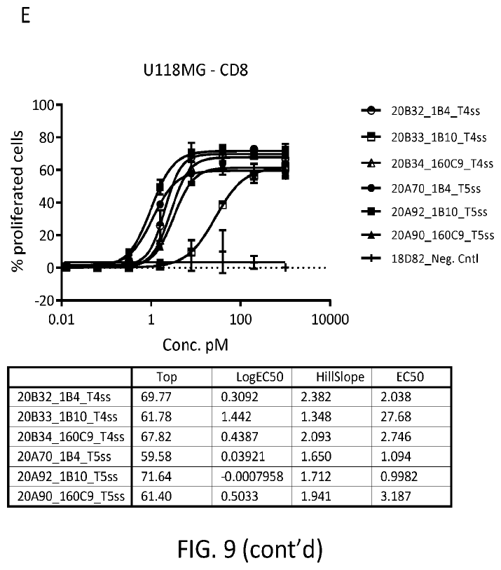

RPMI7951, U118MG, U87MG, or A431 (LRRC15 negative) tumor cells were prepared at 2E5 cells/mL in media containing RPMI 10% human serum, distributed into a flat-bottom 96-well plate at 1E4 cells/well, and incubated overnight. On the following day, T cells were prepared from fresh PBMCs and labeled with 5 uM CellTrace Violet in PBS, 15 min in the dark. Following three washes in media, 5E4 labeled T cells were added to the wells (5:1 Effector:Target cell ratio). The diluted bispecific binding compounds, or control compounds, were then added to the wells containing the tumor cells and the plates were incubated for 5 days prior to flow cytometry analysis of the cells. Cells were analyzed by flow cytometry with the following primary reagent antibodies: CD3-FITC, CD8-PE, 7AAD-PerCP, CellTrace Violet-PB, CD56-APC, CD4-APC-H7 and appropriate isotype control reagents. The results show that LRRC15xCD3 bispecific antibodies potentiate the proliferation of CD4+ and CD8+ T cells in the presence of LRRC15 positive RPMI7951 cells, but not in the presence of A431 tumor cells, which do not express LRRC15 (FIGS. 9A and 9B). Clone 160C9_T4h shows improved potency vs. 160C9_T1 (FIG. 9) Similar results are obtained when the proliferation assay is prepared with U118MG tumor cells. See FIGS. 9C, 9D and 9E.

### Example 9: Cytokine Assay

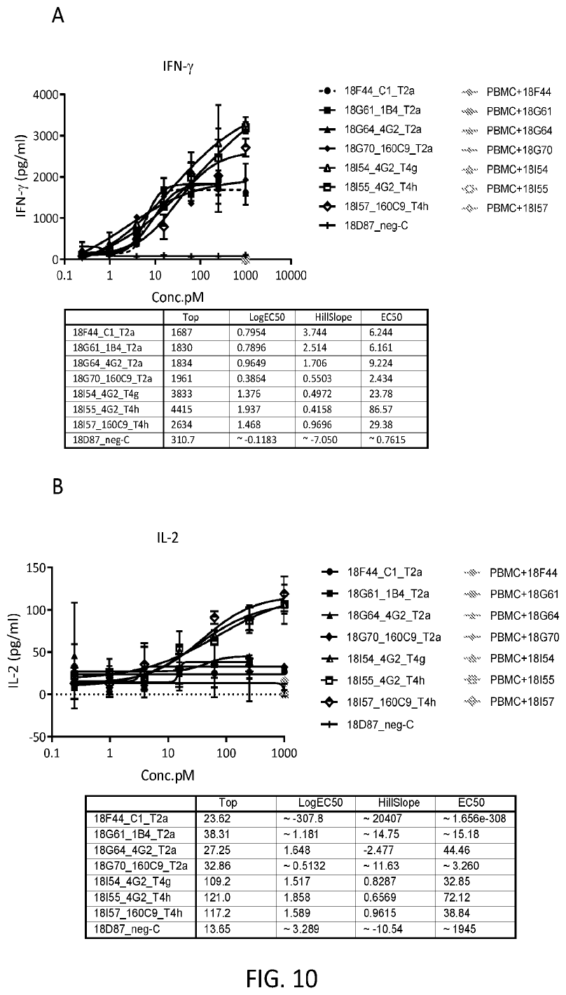

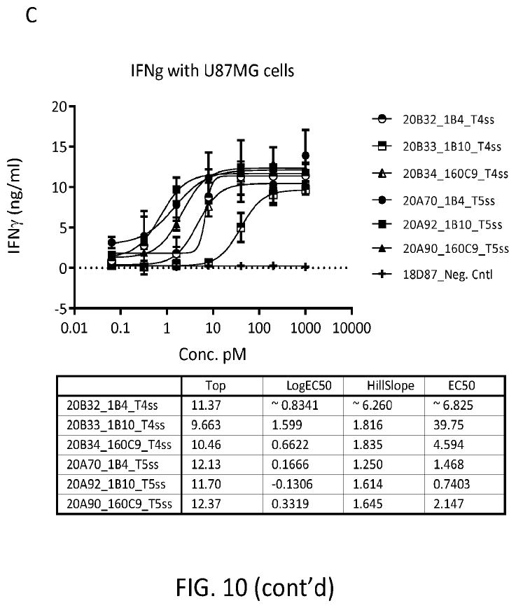

Plates were incubated for 2-5 days in previously described Activation or Proliferation Assays. The supernatants were analyzed by ELISA for IFNγ and IL-2 release, as per manufacturer's protocol (R&D Systems). The results are shown in FIGS. 10A-10C, and demonstrate dose-dependent production of IFNγ and IL-2 in the presence of tumor cells. PBMCs incubated with the highest concentration of compounds did not show IFNγ and IL-2 secretion.

### Example 10: Cytotoxicity Assays

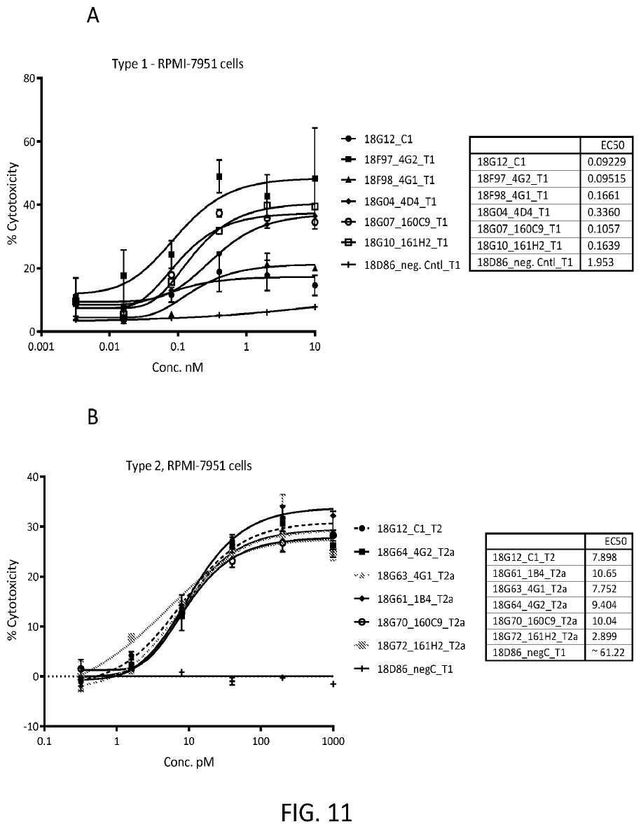

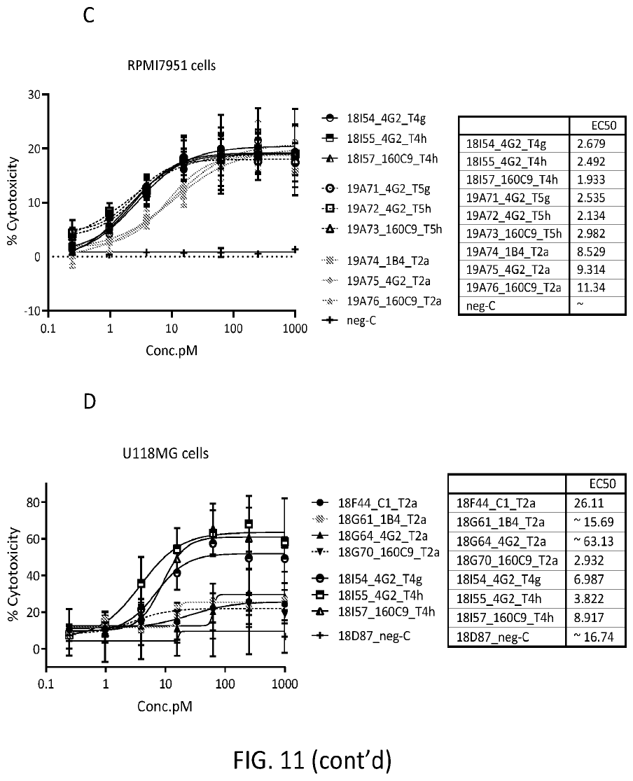

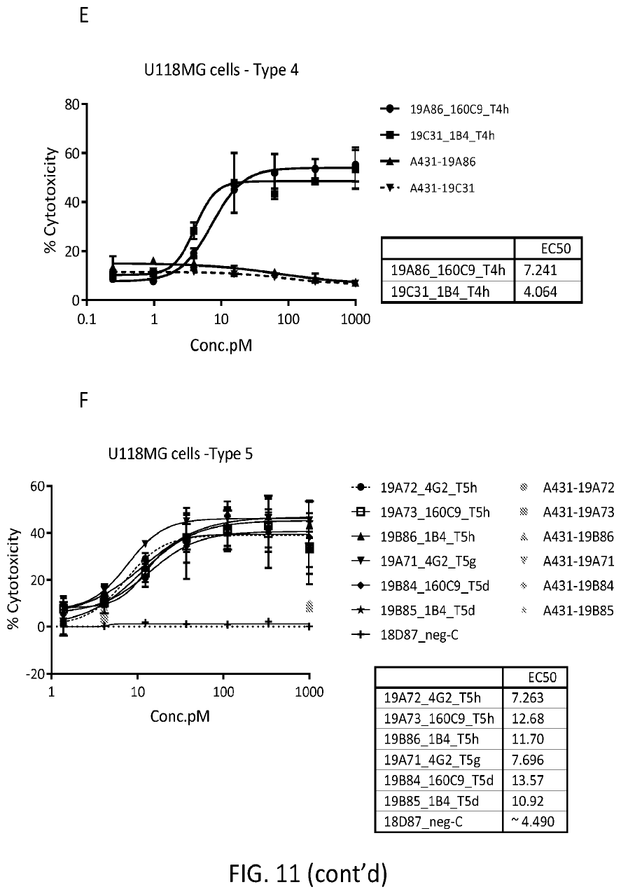

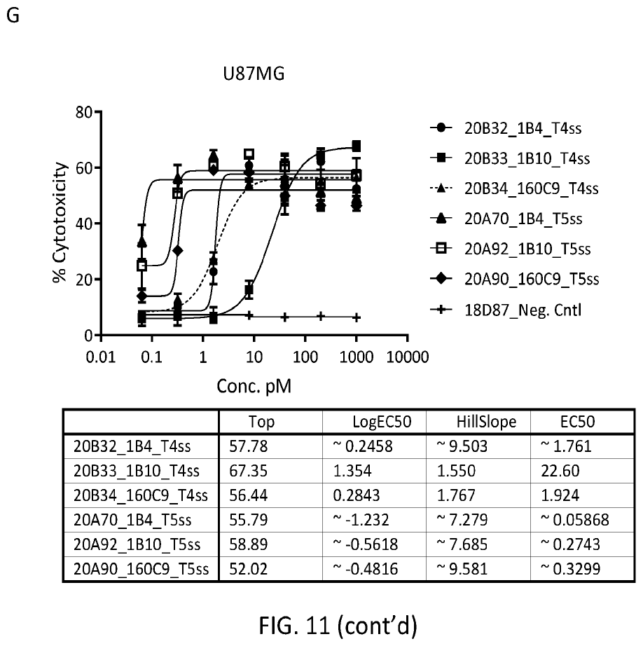

RPMI7951, U118MG, U87MG, or A431 (LRRC15 negative) tumor cells were prepared at 2E5 cells/mL in media containing RPMI 10% human serum, distributed into a flat-bottom 96-well white plate at 1E4 cells/well, and incubated overnight. The following day, CD8+ T cells were purified from PBMCs using a Miltenyi CD8 isolation kit. Serial dilutions starting at 1 nM (final) of the bispecific binding compounds and control binding compounds and 5E4 freshly prepared T cells were then added to the wells. The cell concentrations represented a 1:5 target cell:effector cell ratio. The plates were incubated for 2 days for RPMI7951 cells and 3 days of U118MG and A431 cells. Plates were processed with the CytoTox-Glo kit per manufacturer's instructions and luminescence was analyzed. The results show that LRRC15xCD3 bispecific antibodies potentiate T cell killing of LRRC15 positive tumor cells (FIG. 11). FIGS. 11A-C show examples of T cell directed cytotoxicity of RPMI7951 cells by Type 1, Type 2, Type 4 and Type 5 compounds. Likewise, FIGS. 11D-F show examples of T cell directed cytotoxicity of U118MG cellsby Type 2, Type 4 and Type 5 compounds. Finally, FIG. 11G shows an examples of T cell directed cytotoxicity of U87MG cells.

### Example 11: In Vivo Efficacy Study

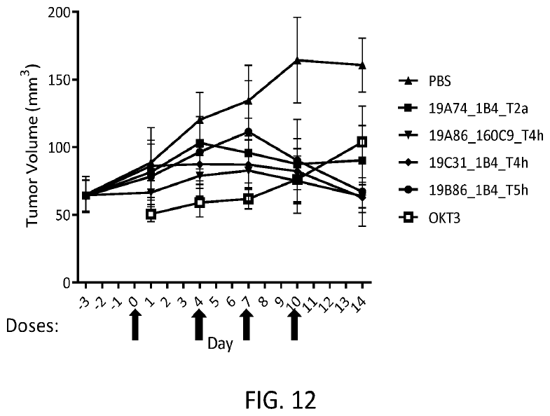

Each immunodeficient NSG mouse (6-9 week-old females from The Jackson Laboratory #005557) were implanted SC with 1E6 U118MG tumor cells and randomized when tumors grew to approximately 60 mm3. Fresh PBMCs from a single donor were implanted IP at 1E7 cells per mouse Animals (8 mice per group) received 4 doses of 1 mg/kg bispecific binding compounds or OKT3 antibody or PBS bi-weekly, beginning 3 days after PBMC implantation Tumors were measured bi-weekly. The results are shown in FIG. 12 and compare tumor volumes of four compounds vs. PBS control. Dosing of all LRRC15xCD3 bispecific antibodies appear to control and/or reduce tumor size.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A multispecific binding compound comprising a first binding unit having binding affinity to LRRC15, and a second binding unit having binding affinity to CD3 epsilon (CD3ε), wherein the first binding unit comprises a heavy chain variable region having at least 95% sequence identity to any one of the sequences of SEQ ID NOs: 24-26 and/or a light chain variable region having at least 95% sequence identity to the sequence of SEQ ID NO: 27.

2. The multispecific binding compound of claim 1, wherein the first binding unit comprises a heavy chain variable region sequence selected from the group consisting of SEQ ID NOs: 24-26 and/or a light chain variable region sequence of SEQ ID NO: 27.

3. The multispecific binding compound of claim 2, wherein the first binding unit comprises:
a heavy chain variable region sequence of SEQ ID NO: 25 and a light chain variable region sequence of SEQ ID NO: 27.

4. The multispecific binding compound of any one of claims 1-3, wherein the second binding unit comprises a heavy chain variable region having at least 95% sequence identity to any one of the sequences of SEQ ID NOs: 28-80 and/or a light chain variable region having at least 95% sequence identity to any one of the sequences of SEQ ID NOs: 81-132.

5. The multispecific binding compound of claim 4, wherein the second binding unit comprises a heavy chain variable region sequence selected from the group consisting of SEQ ID NOs: 28-80 and/or a light chain variable region sequence selected from the group consisting of SEQ ID NOs: 81-132.

6. The multispecific binding compound of claim 5, wherein the second binding unit comprises:
(a) a heavy chain variable region sequence of SEQ ID NO: 55 and a light chain variable region sequence of SEQ ID NO: 107; or (b) a heavy chain variable region sequence of SEQ ID NO: 28 and a light chain variable region sequence of SEQ ID NO: 81.

7. The multispecific binding compound of any one of claims 1-6, wherein the second binding unit comprises a single chain Fv (scFv) comprising a first variable region sequence, a second variable region sequence, and a linker sequence that links the first variable region sequence to the second variable region sequence.

8. The multispecific binding compound of claim 7, wherein the linker sequence comprises the sequence of SEQ ID NO: 229.

9. A multispecific binding compound comprising a first light chain polypeptide (L1), a first heavy chain polypeptide (H1), and a second heavy chain polypeptide (H2), wherein:
L1 comprises a variable region sequence (L1V_{L}) and a constant region sequence (L1C_{L});
H1 comprises a variable region sequence (H1V_{H}), a C_{H}1 constant region sequence (H1 C_{H}1), a C_{H}2 constant region sequence (H1C_{H}2), and a C_{H}3 constant region sequence (H1C_{H}3); and
H2 comprises:
a single chain Fv (H2scFv) comprising a first variable region sequence (H2scFv_{H}), a second variable region sequence (H2scFv_{L}), and a linker sequence that links the H2scFv_{H} sequence to the H2scFv_{L} sequence;
a C_{H}2 constant region sequence (H2C_{H}2); and
a C_{H}3 constant region sequence (H2C_{H}3);
wherein:
the L1V_{L} and H1V_{H} sequences together forma binding unit having binding affinity to LRRC15;
the H2scFv has binding affinity to CD3ε;
the L1C_{L} and H1 C_{H}1 sequences are optionally connected by a disulfide bond;
the H1 and H2 polypeptide chains optionally comprise a hinge region where the H1 and H2 polypeptide chains are optionally connected by at least one disulfide bond; and
the H1 C_{H}3 and H2C_{H}3 sequences comprise an asymmetric interface that facilitates proper pairing between the H1 and H2 polypeptide chains.

10. The multispecific binding compound of claim 9, wherein the L1V_{L} sequence comprises a sequence having at least 95% sequence identity to the sequence of SEQ ID NO: 27.

11. The multispecific binding compound of claim 10, wherein the L1V_{L} sequence comprises the sequence of SEQ ID NO: 27.

12. The multispecific binding compound of claim 9, wherein the H1V_{H} sequence comprises a sequence having at least 95% sequence identity to any one of the sequences of SEQ ID NOs: 24-26.

13. The multispecific binding compound of claim 12, wherein the H1V_{H} sequence is selected from the group consisting of SEQ ID NOs: 24-26.

14. The multispecific binding compound of claim 9, wherein the H2scFv comprises a sequence having at least 95% sequence identity to any one of the sequences of SEQ ID NOs: 133-185.

15. The multispecific binding compound of claim 14, wherein the H2scFv comprises a sequence selected from the group consisting of SEQ ID NOs: 133-185.

16. A multispecific binding compound comprising a first light chain polypeptide (L1), a first heavy chain polypeptide (H1), a second heavy chain polypeptide (H2), and a second light chain polypeptide (L2), wherein:
L1 comprises a variable region sequence (L1V_{L}) and a constant region sequence (L1C_{L});
H1 comprises a variable region sequence (H1V_{H}), a C_{H}1 constant region sequence (H1C_{H}1), a C_{H}2 constant region sequence (H1C_{H}2), a C_{H}3 constant region sequence (H1C_{H}3), and a single chain Fv (H1scFv) comprising a first variable region sequence (H1scFv_{H}), a second variable region sequence (H1scFv_{L}), and a linker sequence that links the H1scFv_{H} sequence to the H1scFv_{L} sequence; and
H2 comprises a variable region sequence (H2V_{H}), a C_{H}1 constant region sequence (H2C_{H}1), a C_{H}2 constant region sequence (H2C_{H}2), a C_{H}3 constant region sequence (H2C_{H}3), and a single chain Fv (H2scFv) comprising a first variable region sequence (H2scFv_{H}), a second variable region sequence (H2scFv_{L}), and a linker sequence that links the H2scFv_{H} sequence to the H2scFv_{L} sequence; and
L2 comprises a variable region sequence (L2V_{L}) and a constant region sequence (L2C_{L});
wherein:
the L1V_{L} and H1V_{H} sequences together forma binding unit having binding affinity to LRRC15;
the L2V_{L} and H2V_{H} sequences together form a binding unit having binding affinity to LRRC15;
the H1scFv and the H2scFv have binding affinity to CD3ε;
the L1C_{L} and H1C_{H}1 sequences are optionally connected by a disulfide bond;
the L2C_{L} and H2C_{H}1 sequences are optionally connected by a disulfide bond;
the H1 and H2 polypeptide chains optionally comprise a hinge region where the H1 and H2 polypeptide chains are optionally connected by at least one disulfide bond.

17. The multispecific binding compound of claim 16, wherein L1 and L2 comprise identical sequences.

18. The multispecific binding compound of claim 16, wherein H1 and H2 comprise identical sequences.

19. The multispecific binding compound of claim 16, wherein L1 and L2 comprise different sequences.

20. The multispecific binding compound of claim 16, wherein H1 and H2 comprise different sequences.

21. The multispecific binding compound of claim 16, wherein the L1V_{L} sequence comprises a sequence having at least 95% sequence identity to the sequence of SEQ ID NO: 27.

22. The multispecific binding compound of claim 21, wherein the L1V_{L} sequence comprises the sequence of SEQ ID NO: 27.

23. The multispecific binding compound of claim 16, wherein the H1V_{H} sequence comprises a sequence having at least 95% sequence identity to any one of the sequences of SEQ ID NOs: 24-26.

24. The multispecific binding compound of claim 23, wherein the H1V_{H} sequence is selected from the group consisting of SEQ ID NOs: 24-26.

25. The multispecific binding compound of claim 16, wherein the H1scFv comprises a sequence having at least 95% sequence identity to any one of the sequences of SEQ ID NOs: 133-185.

26. The multispecific binding compound of claim 25, wherein the H1scFv comprises a sequence selected from the group consisting of SEQ ID NOs: 133-185.

27. The multispecific binding compound of claim 16, wherein the L2V_{L} sequence comprises a sequence having at least 95% sequence identity the sequence of SEQ ID NO: 27.

28. The multispecific binding compound of claim 27, wherein the L2V_{L} sequence comprises the sequence of SEQ ID NO: 27.

29. The multispecific binding compound of claim 16, wherein the H2V_{H} sequence comprises a sequence having at least 95% sequence identity to any one of the sequences of SEQ ID NOs: 24-26.

30. The multispecific binding compound of claim 29, wherein the H2V_{H} sequence is selected from the group consisting of SEQ ID NOs: 24-26.

31. The multispecific binding compound of claim 16, wherein the H2scFv comprises a sequence having at least 95% sequence identity to any one of the sequences of SEQ ID NOs: 133-185.

32. The multispecific binding compound of claim 31, wherein the H2scFv comprises a sequence selected from the group consisting of SEQ ID NOs: 133-185.

33. The multispecific binding compound of claim 16, wherein H1 comprises a sequence order, proceeding from an N-terminus to a C-terminus, as follows: H1V_{H}, H1C_{H}1, H1C_{H}2, H1C_{H}3, H1scFv.

34. The multispecific binding compound of claim 16, wherein H2 comprises a sequence order, proceeding from an N-terminus to a C-terminus, as follows: H2V_{H}, H2C_{H}1, H2C_{H}2, H2C_{H}3, H2scFv.

35. The multispecific binding compound of claim 16, wherein H1 comprises a sequence order, proceeding from an N-terminus to a C-terminus, as follows: H1V_{H}, H1C_{H}1, H1scFv, H1C_{H}2, H1C_{H}3.

36. The multispecific binding compound of claim 16, wherein H2 comprises a sequence order, proceeding from an N-terminus to a C-terminus, as follows: H2V_{H}, H2C_{H}1, H2scFv, H2C_{H}2, H2C_{H}3.

37. A multispecific binding compound comprising a first light chain polypeptide (L1), a first heavy chain polypeptide (H1), a second heavy chain polypeptide (H2), and a second light chain polypeptide (L2), wherein:
L1 comprises a variable region sequence (L1V_{L}) and a constant region sequence (L1C_{L});
H1 comprises a variable region sequence (H1V_{H}), a C_{H}1 constant region sequence (H1C_{H}1), a C_{H}2 constant region sequence (H1C_{H}2), and a C_{H}3 constant region sequence (H1C_{H}3);
H2 comprises a variable region sequence (H2V_{H}), a C_{H}1 constant region sequence (H2C_{H}1), a C_{H}2 constant region sequence (H2C_{H}2), a C_{H}3 constant region sequence (H2C_{H}3), and a single chain Fv (H2scFv) comprising a first variable region sequence (H2scFv_{H}), a second variable region sequence (H2scFv_{L}), and a linker sequence that links the H2scFv_{H} sequence to the H2scFv_{L} sequence; and
L2 comprises a variable region sequence (L2V_{L}) and a constant region sequence (L2C_{L});
wherein:
the L1V_{L} and H1V_{H} sequences together forma binding unit having binding affinity to LRRC15;
the L2V_{L} and H2V_{H} sequences together form a binding unit having binding affinity to LRRC15;
the H2scFv has binding affinity to CD3ε;
the L1C_{L} and H1C_{H}1 sequences are optionally connected by a disulfide bond;
the L2C_{L} and H2C_{H}1 sequences are optionally connected by a disulfide bond;
the H1 and H2 polypeptide chains optionally comprise a hinge region where the H1 and H2 polypeptide chains are optionally connected by at least one disulfide bond; and
the H1C_{H}3 and H2C_{H}3 sequences comprise an asymmetric interface that facilitates proper pairing between the H1 and H2 polypeptide chains.

38. The multispecific binding compound of claim 37, wherein L1 and L2 comprise identical sequences.

39. The multispecific binding compound of claim 37, wherein L1 and L2 comprise different sequences.

40. The multispecific binding compound of claim 37, wherein the L1V_{L} sequence comprises a sequence having at least 95% sequence identity to the sequence of SEQ ID NO: 27.

41. The multispecific binding compound of claim 40, wherein the L1V_{L} sequence comprises the sequence of SEQ ID NO: 27.

42. The multispecific binding compound of claim 37, wherein the H1V_{H} sequence comprises a sequence having at least 95% sequence identity to any one of the sequences of SEQ ID NOs: 24-26.

43. The multispecific binding compound of claim 42, wherein the H1V_{H} sequence is selected from the group consisting of SEQ ID NOs: 24-26.

44. The multispecific binding compound of claim 37, wherein the H2scFv comprises a sequence having at least 95% sequence identity to any one of the sequences of SEQ ID NOs: 133-185.

45. The multispecific binding compound of claim 44, wherein the H2scFv comprises a sequence selected from the group consisting of SEQ ID NOs: 133-185.

46. The multispecific binding compound of claim 37, wherein the L2V_{L} sequence comprises a sequence having at least 95% sequence identity to the sequence of SEQ ID NO: 27.

47. The multispecific binding compound of claim 46, wherein the L2V_{L} sequence comprises the sequence of SEQ ID NO: 27.

48. The multispecific binding compound of claim 37, wherein the H2V_{H} sequence comprises a sequence having at least 95% sequence identity to any one of the sequences of SEQ ID NOs: 24-26.

49. The multispecific binding compound of claim 48, wherein the H2V_{H} sequence is selected from the group consisting of SEQ ID NOs: 24-26.

50. The multispecific binding compound of claim 37, wherein H1 comprises a sequence order, proceeding from an N-terminus to a C-terminus, as follows: H1 V_{H}, H1C_{H}1, H1C_{H}2, H1C_{H}3.

51. The multispecific binding compound of claim 37, wherein H2 comprises a sequence order, proceeding from an N-terminus to a C-terminus, as follows: H2V_{H}, H2C_{H}1, H2C_{H}2, H2C_{H}3, H2scFv.

52. The multispecific binding compound of claim 37, wherein H2 comprises a sequence order, proceeding from an N-terminus to a C-terminus, as follows: H2V_{H}, H2C_{H}1, H2scFv, H2C_{H}2, H2C_{H}3.

53. A multispecific binding compound comprising a first light chain polypeptide (L1), a first heavy chain polypeptide (H1), and a second heavy chain polypeptide (H2), wherein:
L1 comprises a sequence having at least 95% sequence identity to the sequence of SEQ ID NO: 194;
H1 comprises a sequence having at least 95% sequence identity to the sequence of SEQ ID NO: 201; and
H2 comprises a sequence having at least 95% sequence identity to any one of the sequences of SEQ ID NOs: 224 to 228, or 232.

54. A multispecific binding compound comprising a first light chain polypeptide (L1), a first heavy chain polypeptide (H1), and a second heavy chain polypeptide (H2), wherein:
L1 comprises the sequence of SEQ ID NO: 194;
H1 comprises the sequence of SEQ ID NO: 201; and
H2 comprises a sequence selected from the group consisting of SEQ ID NOs: 224 to 228, or 232.

55. A multispecific binding compound comprising a first light chain polypeptide (L1), a first heavy chain polypeptide (H1), and a second heavy chain polypeptide (H2), wherein:
L1 comprises SEQ ID NO: 194;
H1 comprises SEQ ID NO: 201; and
H2 comprises SEQ ID NO: 225.

56. A multispecific binding compound comprising a first light chain polypeptide (L1), a first heavy chain polypeptide (H1), a second heavy chain polypeptide (H2), and a second light chain polypeptide (L2), wherein:
L1 comprises a sequence having at least 95% sequence identity to the sequences of SEQ ID NO: 194;
H1 comprises a sequence having at least 95% sequence identity to any one of the sequences of SEQ ID NOs: 195-223, 231, 233-240;
H2 comprises a sequence having at least 95% sequence identity to any one of the sequences of SEQ ID NOs: 195-223, 231, 233-240; and
L2 comprises a sequence having at least 95% sequence identity to the sequence of SEQ ID NO: 194.

57. A multispecific binding compound comprising a first light chain polypeptide (L1), a first heavy chain polypeptide (H1), a second heavy chain polypeptide (H2), and a second light chain polypeptide (L2), wherein:
L1 comprises the sequence of SEQ ID NO: 194;
H1 comprises a sequence selected from the group consisting of SEQ ID NOs: 195-223, 231, 233-240;
H2 comprises a sequence selected from the group consisting of SEQ ID NOs: 195-223, 231, 233-240; and
L2 comprises the sequence of SEQ ID NO: 194.

58. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises a sequence selected from the group consisting of SEQ ID NOs: 195 and 198;
H2 comprises a sequence selected from the group consisting of SEQ ID NOs: 195 and 198; and
L2 comprises SEQ ID NO: 194.

59. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises a sequence selected from the group consisting of SEQ ID NOs: 196 and 199;
H2 comprises a sequence selected from the group consisting of SEQ ID NOs: 196 and 199; and
L2 comprises SEQ ID NO: 194.

60. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises a sequence selected from the group consisting of SEQ ID NOs: 197 and 200;
H2 comprises a sequence selected from the group consisting of SEQ ID NOs: 197 and 200; and
L2 comprises SEQ ID NO: 194.

61. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises a sequence selected from the group consisting of SEQ ID NOs: 233 and 234;
H2 comprises a sequence selected from the group consisting of SEQ ID NOs: 233 and 234; and
L2 comprises SEQ ID NO: 194.

62. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises SEQ ID NO: 201;
H2 comprises a sequence selected from the group consisting of SEQ ID NOs: 202, 206, 209, and 212; and
L2 comprises SEQ ID NO: 194.

63. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises SEQ ID NO: 201;
H2 comprises a sequence selected from the group consisting of SEQ ID NOs: 203, 207, 210, and 213; and
L2 comprises SEQ ID NO: 194.

64. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises SEQ ID NO: 201;
H2 comprises a sequence selected from the group consisting of SEQ ID NOs: 204, 208, 211, and 214; and
L2 comprises SEQ ID NO: 194.

65. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises SEQ ID NO: 201;
H2 comprises SEQ ID NO: 205; and
L2 comprises SEQ ID NO: 194.

66. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises SEQ ID NO: 231;
H2 comprises a sequence selected from the group consisting of SEQ ID NOs: 235, 236, and 237; and
L2 comprises SEQ ID NO: 194.

67. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises a sequence selected from the group consisting of SEQ ID NOs: 215, 219, 221, and 239;
H2 comprises a sequence selected from the group consisting of SEQ ID NOs: 215, 219, 221, and 239; and
L2 comprises SEQ ID NO: 194.

68. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises a sequence selected from the group consisting of SEQ ID NOs: 216, 220, 222, and 240;
H2 comprises a sequence selected from the group consisting of SEQ ID NOs: 216, 220, 222, and 240; and
L2 comprises SEQ ID NO: 194.

69. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises a sequence selected from the group consisting of SEQ ID NOs: 217 and 223;
H2 comprises a sequence selected from the group consisting of SEQ ID NOs: 217 and 223; and
L2 comprises SEQ ID NO: 194.

70. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises SEQ ID NO: 218;
H2 comprises SEQ ID NO: 218; and
L2 comprises SEQ ID NO: 194.

71. A multispecific binding compound comprising a first light chain polypeptide (L1), a first heavy chain polypeptide (H1), a second heavy chain polypeptide (H2), and a second light chain polypeptide (L2), wherein:
L1 comprises SEQ ID NO: 194;
H1 comprises SEQ ID NO: 238;
H2 comprises SEQ ID NO: 238; and
L2 comprises SEQ ID NO: 194.

72. A pharmaceutical composition comprising the multispecific binding compound of any one of claims 1-68.

73. A method of treatment, comprising administering to an individual in need an effective dose of the multispecific binding compound of any one of claims 1-68, or the pharmaceutical composition of claim 69.

74. A method for the treatment of a disorder characterized by expression of LRRC15, comprising administering to a subject with said disorder a multispecific binding compound of any one of claims 1-68, or the pharmaceutical composition of claim 69.

75. Use of a multispecific binding compound of any one of claims 1-68, in the preparation of a medicament for the treatment of a disorder characterized by expression of LRRC15.

76. A multispecific binding compound of any one of claims 1-68, for use in the treatment of a disorder characterized by expression of LRRC15.

77. The method, use, or multispecific binding compound of any one of claims 71-73, wherein the disorder is selected from the group consisting of: sarcoma, breast cancer, lung cancer, ovarian cancer, pancreatic cancer, and large B cell lymphoma.

78. A polynucleotide encoding a multispecific binding compound of any one of claims 1-68.

79. A vector comprising the polynucleotide of claim 75.

80. A cell comprising the vector of claim 76.

81. A method of producing a multispecific binding compound of any one of claims 1-68, comprising growing a cell according to claim 77 under conditions permissive for expression of the multispecific binding compound, and isolating the multispecific binding compound.


SEQUENCE LISTING (244 sequences)

SEQ ID NO: 1 - protein, 5 aa, Artificial Sequence
  source: /note=“Description of Artificial Sequence Synthetic peptide”
SYWIE

SEQ ID NO: 2 - protein, 5 aa, Artificial Sequence
  source: /note=“Description of Artificial Sequence Synthetic peptide”
TYAMN

SEQ ID NO: 3 - protein, 17 aa, Artificial Sequence
  source: /note=“Description of Artificial Sequence Synthetic peptide”
EILPGSDTTNYNEKFKD

SEQ ID NO: 4 - protein, 19 aa, Artificial Sequence
  source: /note=“Description of Artificial Sequence Synthetic peptide”
RIRSKYNNYATYYADSVKD

SEQ ID NO: 5 - protein, 19 aa, Artificial Sequence
  source: /note=“Description of Artificial Sequence Synthetic peptide”
RIRSKANNYATYYADSVKG

SEQ ID NO: 6 - protein, 19 aa, Artificial Sequence
  source: /note=“Description of Artificial Sequence Synthetic peptide”
RIRSKYNNYATYYADPVKG

SEQ ID NO: 7 - protein, 19 aa, Artificial Sequence
  source: /note=“Description of Artificial Sequence Synthetic peptide”
RIRSKYNNYATYYADSVKG

SEQ ID NO: 8 - protein, 19 aa, Artificial Sequence
  source: /note=“Description of Artificial Sequence Synthetic peptide”
RIRSKYNNYATYYAASVKD

SEQ ID NO: 9 - protein, 19 aa, Artificial Sequence
  source: /note=“Description of Artificial Sequence Synthetic peptide”
RIRSKTNNYATYYADSVKD

SEQ ID NO: 10 - protein, 19 aa, Artificial Sequence
  source: /note=“Description of Artificial Sequence Synthetic peptide”
RIRSKSNNYATYYAASVKD

SEQ ID NO: 11 - protein, 19 aa, Artificial Sequence
  source: /note=“Description of Artificial Sequence Synthetic peptide”
RIRSKSNNYATYYADSVKD

SEQ ID NO: 12 - protein, 19 aa, Artificial Sequence
  source: /note=“Description of Artificial Sequence Synthetic peptide”
RIRSKYNNYATYYADPVKD

SEQ ID NO: 13 - protein, 11 aa, Artificial Sequence
  source: /note=“Description of Artificial Sequence Synthetic peptide”
DRGNYRAWFGY

SEQ ID NO: 14 - protein, 14 aa, Artificial Sequence
  source: /note=“Description of Artificial Sequence Synthetic peptide”
HGNFGNSYVSWFAY

SEQ ID NO: 15 - protein, 11 aa, Artificial Sequence
  source: /note=“Description of Artificial Sequence Synthetic peptide”
RASQDISNYLN

SEQ ID NO: 16 - protein, 14 aa, Artificial Sequence
  source: /note=“Description of Artificial Sequence Synthetic peptide”
GSSTGAVTTSNYAN

SEQ ID NO: 17 - protein, 14 aa, Artificial Sequence
  source: /note=“Description of Artificial Sequence Synthetic peptide”
RSSTGAVTTSNYAN

SEQ ID NO: 18 - protein, 14 aa, Artificial Sequence
  source: /note=“Description of Artificial Sequence Synthetic peptide”
GSSTGAVTNSNYAN

SEQ ID NO: 19 - protein, 7 aa, Artificial Sequence
  source: /note=“Description of Artificial Sequence Synthetic peptide”
YTSRLHS

SEQ ID NO: 20 - protein, 7 aa, Artificial Sequence
  source: /note=“Description of Artificial Sequence Synthetic peptide”
GTNKRAP

SEQ ID NO: 21 - protein, 7 aa, Artificial Sequence
  source: /note=“Description of Artificial Sequence Synthetic peptide”
GTNKRAS

SEQ ID NO: 22 - protein, 9 aa, Artificial Sequence
  source: /note=“Description of Artificial Sequence Synthetic peptide”
QQGEALPWT

SEQ ID NO: 23 - protein, 9 aa, Artificial Sequence
  source: /note=“Description of Artificial Sequence Synthetic peptide”
ALWYSNLWV

SEQ ID NO: 24 - protein, 120 aa, Artificial Sequence
  source: /note=“Description of Artificial Sequence Synthetic polypeptide”
QVQLVQSGAEVKKPGASVKVSCKASGYTFTSYWIEWVRQAPGQGLEWMGEILPGSDTTNYNEKFKDRVTMTRDTSTSTVYMELSSLRSEDTAVYYCARDRGNYRAWFGYWGQGTLVTVSS

SEQ ID NO: 25 - protein, 120 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSS

SEQ ID NO: 26 - protein, 120 aa, Artificial Sequence
  source: /note=“Description of Artificial Sequence Synthetic polypeptide”
QVQLVQSGAEVKKPGASVKVSCKASGYKFSSYWIEWVRQAPGQGLEWIGEILPGSDTTNYNEKFKDKATFTSDTSTSTAYMELSSLRSEDTAVYYCARDRGNYRAWFGYWGQGTLVTVSS

SEQ ID NO: 27 - protein, 107 aa, Artificial Sequence
  source: /note=“Description of Artificial Sequence Synthetic polypeptide”
DIQMTQSPSSLSASVGDRVTITCRASQDISNYLNWYQQKPGKAPKFLIYYTSRLHSGVPSRFSGSGSGTDYTLTISSLQPEDFATYYCQQGEALPWTFGQGTKVEIK

SEQ ID NO: 28 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSS

SEQ ID NO: 29 - protein, 125 aa, Artificial Sequence
  source: /note=“Description of Artificial Sequence Synthetic polypeptide”
EVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSS

SEQ ID NO: 30 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADPVKGRFTISRDDAQNTLYLQMNNLKAEDTAVYYCVRHGNFGNSYVSWFAYWGQGTLVTVSS

SEQ ID NO: 31 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKNTLYLQMNNLRAEDTAVYYCVRHGNFGNSYVSWFAYWGQGTLVTVSS

SEQ ID NO: 32 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSKNILYLQMNNLKAEDTAVYYCVRHGNFGNSYVSWFAYWGQGTLVTVSS

SEQ ID NO: 33 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNTLYLQMNNLRAEDTAVYYCVRHGNFGNSYVSWFAYWGQGTLVTVSS

SEQ ID NO: 34 - protein, 125 aa, Artificial Sequence; identical to SEQ ID NO: 33
  source: /note=“Description of Artificial Sequence Synthetic polypeptide”

SEQ ID NO: 35 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYAASVKDRFTISRDDSKNTLYLQMNNLRAEDTAVYYCVRHGNFGNSYVSWFAYWGQGTLVTVSS

SEQ ID NO: 36 - protein, 125 aa, Artificial Sequence; identical to SEQ ID NO: 33
  source: /note=“Description of Artificial Sequence Synthetic polypeptide”

SEQ ID NO: 37 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKNTLYLQMNNLKAEDTAVYYCVRHGNFGNSYVSWFAYWGQGTLVTVSS

SEQ ID NO: 38 - protein, 125 aa, Artificial Sequence; identical to SEQ ID NO: 37
  source: /note=“Description of Artificial Sequence Synthetic polypeptide”

SEQ ID NO: 39 - protein, 125 aa, Artificial Sequence
  source: /note=“Description of Artificial Sequence Synthetic polypeptide”
EVQLLESGGGLVQPGGSLKLSCAASGFTFNTYAMNWVRQAPGKGLEWVGRIRSKANNYATYYADSVKGRFTISRDDSKNILYLQMNSLRAEDTAVYYCVRHGNFGNSYVSWFAYWGQGTLVTVSS

SEQ ID NO: 40 - protein, 125 aa, Artificial Sequence; identical to SEQ ID NO: 39
  source: /note=“Description of Artificial Sequence Synthetic polypeptide”

SEQ ID NO: 41 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNTLYLQMNNLKAEDTAVYYCVRHGNFGNSYVSWFAYWGQGTLVTVSS

SEQ ID NO: 42 - protein, 125 aa, Artificial Sequence; identical to SEQ ID NO: 41
  source: /note=“Description of Artificial Sequence Synthetic polypeptide”

SEQ ID NO: 43 - protein, 125 aa, Artificial Sequence
  source: /note=“Description of Artificial Sequence Synthetic polypeptide”
EVRLVESGGGLVQPGGSLKLSCAASGFTFNTYAMNWVRQAPGKGLEWVGRIRSKTNNYATYYADSVKDRFTISRDDAQNILYLQMNNLRAEDTAVYYCVRHGNFGNSYVSWFAYWGQGTLVTVSS

SEQ ID NO: 44 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKSNNYATYYAASVKDRFTISRDDSKNSLYLQMNNLKAEDTAVYYCVRHGNFGNSYVSWFAYWGQGTLVTVSS

SEQ ID NO: 45 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSKNTLYLQMNNLKTEDTAVYYCVRHGNFGNSYVSWFAYWGQGTLVTVSS

SEQ ID NO: 46 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKSNNYATYYADSVKDRFTISRDDSKNILYLQMNNLKAEDTAVYYCVRHGNFGNSYVSWFAYWGQGTLVTVSS

SEQ ID NO: 47 - protein, 125 aa, Artificial Sequence; identical to SEQ ID NO: 46
  source: /note=“Description of Artificial Sequence Synthetic polypeptide”

SEQ ID NO: 48 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDAQNILYLQMNNLKTEDTAVYYCVRHGNFGNSYVSWFAYWGQGTLVTVSS

SEQ ID NO: 49 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSQSILYLQMNNLRAEDTAVYYCVRHGNFGNSYVSWFAYWGQGTLVTVSS

SEQ ID NO: 50 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSQNILYLQMNNLKAEDTAVYYCVRHGNFGNSYVSWFAYWGQGTLVTVSS

SEQ ID NO: 51 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADPVKGRFTISRDDAKNTLYLQMNSLKAEDTAVYYCVRHGNFGNSYVSWFAYWGQGTLVTVSS

SEQ ID NO: 52 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKSTLYLQMNNLRTEDTAVYYCVRHGNFGNSYVSWFAYWGQGTLVTVSS

SEQ ID NO: 53 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDAQNILYLQMNNLKTEDTAVYYCVRHGNFGNSYVSWFAYWGQGTLVTVSS

SEQ ID NO: 54 - protein, 125 aa, Artificial Sequence
  source: /note=“Description of Artificial Sequence Synthetic polypeptide”
EVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSS

SEQ ID NO: 55 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSS

SEQ ID NO: 56 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKGRFTISRDDAKSTLYLQMNSLRTEDTAVYYCVRHGNFGNSYVSWFAYWGQGTLVTVSS

SEQ ID NO: 57 - protein, 125 aa, Artificial Sequence
  source: /note=“Description of Artificial Sequence Synthetic polypeptide”
EVQLLESGGGLVQPGGSLKLSCAASGFTFNTYAMNWVRQAPGKGLEWVARIRSKYNNYATYYADSVKDRFTISRDDSQNILYLQMNNLKTEDTAVYYCVRHGNFGNSYVSWFAYWGQGTLVTVSS

SEQ ID NO: 58 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDSKNTLYLQMNSLKTEDTAVYYCVRHGNFGNSYVSWFAYWGQGTLVTVSS

SEQ ID NO: 59 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVARIRSKYNNYATYYADSVKDRFTISRDDSKNILYLQMNSLRTEDTAVYYCVRHGNFGNSYVSWFAYWGQGTLVTVSS

SEQ ID NO: 60 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAQNILYLQMNNLKTEDTAVYYCVRHGNFGNSYVSWFAYWGQGTLVTVSS

SEQ ID NO: 61 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKGRFTISRDDSKSSLYLQMNSLKAEDTAVYYCVRHGNFGNSYVSWFAYWGQGTLVTVSS

SEQ ID NO: 62 - protein, 125 aa, Artificial Sequence; identical to SEQ ID NO: 32
  source: /note=“Description of Artificial Sequence Synthetic polypeptide”

SEQ ID NO: 63 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSQNILYLQMNNLRTEDTAVYYCVRHGNFGNSYVSWFAYWGQGTLVTVSS

SEQ ID NO: 64 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDAKNILYLQMNNLRTEDTAVYYCVRHGNFGNSYVSWFAYWGQGTLVTVSS

SEQ ID NO: 65 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKSILYLQMNNLRAEDTAVYYCVRHGNFGNSYVSWFAYWGQGTLVTVSS

SEQ ID NO: 66 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNILYLQMNNLRTEDTAVYYCVRHGNFGNSYVSWFAYWGQGTLVTVSS

SEQ ID NO: 67 - protein, 125 aa, Artificial Sequence
  source: /note=“Description of Artificial Sequence Synthetic polypeptide”
EVQLLESGGGLVQPGGSLKLSCAASGFTFNTYAMNWVRQAPGKGLEWVGRIRSKYNNYATYYADSVKGRFTISRDDSQNTLYLQMNNLKTEDTAVYYCVRHGNFGNSYVSWFAYWGQGTLVTVSS

SEQ ID NO: 68 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADPVKGRFTISRDDSKSTLYLQMNNLRTEDTAVYYCVRHGNFGNSYVSWFAYWGQGTLVTVSS

SEQ ID NO: 69 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKSTLYLQMNNLRAEDTAVYYCVRHGNFGNSYVSWFAYWGQGTLVTVSS

SEQ ID NO: 70 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKGRFTISRDDSQSTLYLQMNSLRAEDTAVYYCVRHGNFGNSYVSWFAYWGQGTLVTVSS

SEQ ID NO: 71 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVARIRSKYNNYATYYADSVKGRFTISRDDSKNTLYLQMNNLKAEDTAVYYCVRHGNFGNSYVSWFAYWGQGTLVTVSS

SEQ ID NO: 72 - protein, 125 aa, Artificial Sequence
  source: /note=“Description of Artificial Sequence Synthetic polypeptide”
EVQLLESGGGLVQPGGSLKLSCAASGFTFNTYAMNWVRQAPGKGLEWVGRIRSKYNNYATYYADSVKDRFTISRDDSKSTLYLQMNNLKAEDTAVYYCVRHGNFGNSYVSWFAYWGQGTLVTVSS

SEQ ID NO: 73 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVSRIRSKYNNYATYYADPVKDRFTISRDDAQNTLYLQMNNLKAEDTAVYYCVRHGNFGNSYVSWFAYWGQGTLVTVSS

SEQ ID NO: 74 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKGRFTISRDDSKNILYLQMNSLKAEDTAVYYCVRHGNFGNSYVSWFAYWGQGTLVTVSS

SEQ ID NO: 75 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDAKSILYLQMNNLKAEDTAVYYCVRHGNFGNSYVSWFAYWGQGTLVTVSS

SEQ ID NO: 76 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSQNTLYLQMNNLKAEDTAVYYCVRHGNFGNSYVSWFAYWGQGTLVTVSS

SEQ ID NO: 77 - protein, 125 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKGRFTISRDDAKSTLYLQMNNLKTEDTAVYYCVRHGNFGNSYVSWFAYWGQGTLVTVSS

SEQ ID NO: 78 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDSQSILYLQMNNLKAEDTAVYYCVRHGNFGNSYVSWFAYWGQGTLVTVSS

SEQ ID NO: 79 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDAKNSLYLQMNNLKAEDTAVYYCVRHGNFGNSYVSWFAYWGQGTLVTVSS

SEQ ID NO: 80 - protein, 125 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADPVKDRFTISRDDSQNILYLQMNNLKAEDTAVYYCVRHGNFGNSYVSWFAYWGQGTLVTVSS

SEQ ID NO: 81 - protein, 109 aa, Artificial Sequence
  source: /note=“Description of Artificial Sequence Synthetic polypeptide”
QAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVL

SEQ ID NO: 82 - protein, 109 aa, Artificial Sequence
  source: /note=“Description of Artificial Sequence Synthetic polypeptide”
EAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVL

SEQ ID NO: 83 - protein, 109 aa, Artificial Sequence
  source: /note=“Description of Artificial Sequence Synthetic polypeptide”
EAVVTQEPSLTVSPGGTVTLTCRSSTGAVTTSNYANWVQQKPGQAFRGLIGGTNKRAPGVPARFSGSLLGDKAALTIAGAQPEDEAEYYCALWYSNLWVFGGGTKLTVL

SEQ ID NO: 84 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAFRGLIGGTNKRASGVPARFSGSLIGDKAALTIAGAQPEDEAEYYCALWYSNLWVFGGGTKLTVL

SEQ ID NO: 85 - protein, 109 aa, Artificial Sequence
  source: /note=“Description of Artificial Sequence Synthetic polypeptide”
EAVVTQEPSLTVSPGGTVTLTCRSSTGAVTTSNYANWVQQKPGQAFRGLIGGTNKRAPGVPARFSGSLIGDKAALTITGAQPEDEAEYYCALWYSNLWVFGGGTKLTVL

SEQ ID NO: 86 - protein, 109 aa, Artificial Sequence
  source: /note=“Description of Artificial Sequence Synthetic polypeptide”
EAVVTQEPSLTVSPGGTVTLTCRSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 87 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAFRGLIGGTNKRAPGVPARFSGSLLGGKAALTITGAQPEDEAEYYCALWYSNLWVFGGGTKLTVL

SEQ ID NO: 88 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 89 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAFRGLIGGTNKRAPGVPARFSGSLLGDKAALTITGAQPEDEAEYYCALWYSNLWVFGGGTKLTVL

SEQ ID NO: 90 - protein, 109 aa, Artificial Sequence; identical to SEQ ID NO: 88
  source: /note=“Description of Artificial Sequence Synthetic polypeptide”

SEQ ID NO: 91 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAFRGLIGGTNKRAPGVPARFSGSLIGGKAALTIAGAQPEDEAEYYCALWYSNLWVFGGGTKLTVL

SEQ ID NO: 92 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAPRGLIGGTNKRAPGVPARFSGSLLGGKAALTLAGAQPEDEAEYYCALWYSNLWVFGGGTKLTVL

SEQ ID NO: 93 - protein, 109 aa, Artificial Sequence; identical to SEQ ID NO: 85
  source: /note=“Description of Artificial Sequence Synthetic polypeptide”

SEQ ID NO: 94 - protein, 109 aa, Artificial Sequence; identical to SEQ ID NO: 86
  source: /note=“Description of Artificial Sequence Synthetic polypeptide”

SEQ ID NO: 95 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAPRGLIGGTNKRAPGVPARFSGSLIGDKAALTIAGAQPEDEAEYYCALWYSNLWVFGGGTKLTVL

SEQ ID NO: 96 - protein, 109 aa, Artificial Sequence
  source: /note=“Description of Artificial Sequence Synthetic polypeptide”
QAVVTQEPSLTVSPGGTVTLTCGSSTGAVTNSNYANWVQQKPGQAFRGLIGGTNKRAPGTPARFSGSLLGGKAALTIAGVQPEDEAEYYCALWYSNLWVFGGGTKLTVL

SEQ ID NO: 97 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAPRGLIGGTNKRAPWTPARFSGSLIGDKAALTIAGAQPEDEAEYYCALWYSNLWVFGGGTKLTVL

SEQ ID NO: 98 - protein, 109 aa, Artificial Sequence
  source: /note=“Description of Artificial Sequence Synthetic polypeptide”
EAVVTQEPSLTVSPGGTVTLTCGSSTGAVTTSNYANWVQQKPGQAFRGLIGGTNKRAPGTPARFSGSLIGGKAALTIAGAQPEDEAEYYCALWYSNLWVFGGGTKLTVL

SEQ ID NO: 99 - protein, 109 aa, Artificial Sequence
  source: /note=“Description of Artificial Sequence Synthetic polypeptide”
EAVVTQEPSLTVSPGGTVTLTCGSSTGAVTTSNYANWVQQKPGQAPRGLIGGTNKRAPGTPARFSGSLLGGKAALTLAGAQPEDEAEYYCALWYSNLWVFGGGTKLTVL

SEQ ID NO: 100 - protein, 109 aa, Artificial Sequence
  source: /note=“Description of Artificial Sequence Synthetic polypeptide”
EAVVTQEPSLTVSPGGTVTLTCGSSTGAVTTSNYANWVQQKPGQAFRGLIGGTNKRAPGVPARFSGSLIGDKAALTLAGAQPEDEAEYYCALWYSNLWVFGGGTKLTVL

SEQ ID NO: 101 - protein, 109 aa, Artificial Sequence
  source: /note=“Description of Artificial Sequence Synthetic polypeptide”
EAVVTQEPSLTVSPGGTVTLTCGSSTGAVTTSNYANWVQQKPGQAFRGLIGGTNKRAPGVPARFSGSLIGDKAALTITGAQPEDEAEYYCALWYSNLWVFGGGTKLTVL

SEQ ID NO: 102 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAPRGLIGGTNKRAPGVPARFSGSLIGGKAALTITGVQPEDEAEYYCALWYSNLWVFGGGTKLTVL

SEQ ID NO: 103 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAFRGLIGGTNKRAPGVPARFSGSLIGDKAALTITGAQPEDEAEYYCALWYSNLWVFGGGTKLTVL

SEQ ID NO: 104 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAFRGLIGGTNKRAPGTPARFSGSLIGDKAALTITGAQPEDEAEYYCALWYSNLWVFGGGTKLTVL

SEQ ID NO: 105 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAFRGLIGGTNKRAPGTPARFSGSLLGDKAALTITGAQPEDEAEYYCALWYSNLWVFGGGTKLTVL

SEQ ID NO: 106 - protein, 109 aa, Artificial Sequence
  source: /note=“Description of Artificial Sequence Synthetic polypeptide”
EAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVL

SEQ ID NO: 107 - protein, 109 aa, Artificial Sequence
  source: /note=“Description of Artificial Sequence Synthetic polypeptide”
QAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 108 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAFRGLIGGTNKRAPGVPARFSGSLLGGKAALTLSGAQPEDEAEYYCALWYSNLWVFGGGTKLTVL

SEQ ID NO: 109 - protein, 109 aa, Artificial Sequence; identical to SEQ ID NO: 107
  source: /note=“Description of Artificial Sequence Synthetic polypeptide”

SEQ ID NO: 110 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 111 - protein, 109 aa, Artificial Sequence; identical to SEQ ID NO: 110
  source: /note=“Description of Artificial Sequence Synthetic polypeptide”

SEQ ID NO: 112 - protein, 109 aa, Artificial Sequence; identical to SEQ ID NO: 110
  source: /note=“Description of Artificial Sequence Synthetic polypeptide”

SEQ ID NO: 113 - protein, 109 aa, Artificial Sequence
  source: /note=“Description of Artificial Sequence Synthetic polypeptide”
QAVVTQEPSLTVSPGGTVTLTCRSSTGAVTTSNYANWVQQKPGQAFRGLIGGTNKRAPGVPARFSGSLLGGKAALTLSGAQPEDEAEYYCALWYSNLWVFGGGTKLTVL

SEQ ID NO: 114 - protein, 109 aa, Artificial Sequence
  source: /note=“Description of Artificial Sequence Synthetic polypeptide”
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 115 - protein, 109 aa, Artificial Sequence; identical to SEQ ID NO: 107
  source: /note=“Description of Artificial Sequence Synthetic polypeptide”

SEQ ID NO: 116 - protein, 109 aa, Artificial Sequence; identical to SEQ ID NO: 88
  source: /note=“Description of Artificial Sequence Synthetic polypeptide”

SEQ ID NO: 117 - protein, 109 aa, Artificial Sequence; identical to SEQ ID NO: 88
  source: /note=“Description of Artificial Sequence Synthetic polypeptide”

SEQ ID NO: 118 - protein, 109 aa, Artificial Sequence; identical to SEQ ID NO: 107
  source: /note=“Description of Artificial Sequence Synthetic polypeptide”

SEQ ID NO: 119 - protein, 109 aa, Artificial Sequence; identical to SEQ ID NO: 110
  source: /note=“Description of Artificial Sequence Synthetic polypeptide”

SEQ ID NO: 120 - protein, 109 aa, Artificial Sequence; identical to SEQ ID NO: 110
  source: /note=“Description of Artificial Sequence Synthetic polypeptide”

SEQ ID NO: 121 - protein, 109 aa, Artificial Sequence; identical to SEQ ID NO: 110
  source: /note=“Description of Artificial Sequence Synthetic polypeptide”

SEQ ID NO: 122 - protein, 109 aa, Artificial Sequence; identical to SEQ ID NO: 110
  source: /note=“Description of Artificial Sequence Synthetic polypeptide”

SEQ ID NO: 123 - protein, 109 aa, Artificial Sequence; identical to SEQ ID NO: 88
  source: /note=“Description of Artificial Sequence Synthetic polypeptide”

SEQ ID NO: 124 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 125 - protein, 109 aa, Artificial Sequence
  source: /note=“Description of Artificial Sequence Synthetic polypeptide”
QAVVTQEPSLTVSPGGTVTLTCGSSTGAVTTSNYANWVQQKPGQAPRGLIGGTNKRAPGVPARFSGSLLGDKAALTLSGAQPEDEAEYYCALWYSNLWVFGGGTKLTVL

SEQ ID NO: 126 - protein, 109 aa, Artificial Sequence; identical to SEQ ID NO: 110
  source: /note=“Description of Artificial Sequence Synthetic polypeptide”

SEQ ID NO: 127 - protein, 109 aa, Artificial Sequence; identical to SEQ ID NO: 110
  source: /note=“Description of Artificial Sequence Synthetic polypeptide”

SEQ ID NO: 128 - protein, 109 aa, Artificial Sequence; identical to SEQ ID NO: 108
  source: /note=“Description of Artificial Sequence Synthetic polypeptide”

SEQ ID NO: 129 - protein, 109 aa, Artificial Sequence; identical to SEQ ID NO: 107
  source: /note=“Description of Artificial Sequence Synthetic polypeptide”

SEQ ID NO: 130 - protein, 109 aa, Artificial Sequence; identical to SEQ ID NO: 110
  source: /note=“Description of Artificial Sequence Synthetic polypeptide”

SEQ ID NO: 131 - protein, 109 aa, Artificial Sequence; identical to SEQ ID NO: 88
  source: /note=“Description of Artificial Sequence Synthetic polypeptide”

SEQ ID NO: 132 - protein, 109 aa, Artificial Sequence; identical to SEQ ID NO: 110
  source: /note=“Description of Artificial Sequence Synthetic polypeptide”

SEQ ID NO: 133 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVL

SEQ ID NO: 134 - protein, 250 aa, Artificial Sequence
  source: /note=“Description of Artificial Sequence Synthetic polypeptide”
EVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVL

SEQ ID NO: 135 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADPVKGRFTISRDDAQNTLYLQMNNLKAEDTAVYYCVRHGNFGNSYVSWFAYWGQGTLVTVSSGGGGSGGGGSGGGGSAEAVVTQEPSLTVSPGGTVTLTCRSSTGAVTTSNYANWVQQKPGQAFRGLIGGTNKRAPGVPARFSGSLLGDKAALTIAGAQPEDEAEYYCALWYSNLWVFGGGTKLTVL

SEQ ID NO: 136 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKNTLYLQMNNLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRASGVPARFSGSLIGDKAALTIAGAQPEDEAEYYCALWYSNLWVFGGGTKLTVL

SEQ ID NO: 137 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSKNILYLQMNNLKAEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLIGDKAALTITGVQPEDEAEYYCALWYSNLWVFGGGTKLTVL

SEQ ID NO: 138 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNTLYLQMNNLRAEDTAVYYCVRHGNFGNSYVSWFAYWGQGTLVTVSSGGGGSGGGGSGGGGSAEAVVTQEPSLTVSPGGTVTLTCRSSTGAVTTSNYANWVQQKPGQAFRGLIGGTNKRAPGVPARFSGSLIGDKAALTITGAQPEDEAEYYCALWYSNLWVFGGGTKLTVL

SEQ ID NO: 139 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNTLYLQMNNLRAEDTAVYYCVRHGNFGNSYVSWFAYWGQGTLVTVSSGGGGSGGGGSGGGGSAEAVVTQEPSLTVSPGGTVTLTCRSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 140 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYAASVKDRFTISRDDSKNTLYLQMNNLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRAPGVPARFSGSLLGGKAALTITGAQPEDEAEYYCALWYSNLWVFGGGTKLTVL

SEQ ID NO: 141 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNTLYLQMNNLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 142 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKNTLYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRAPGVPARFSGSLLGDKAALTITGAQPEDEAEYYCALWYSNLWVFGGGTKLTVL

SEQ ID NO: 143 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKNTLYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 144 - protein, 250 aa, Artificial Sequence
  source: /note=“Description of Artificial Sequence Synthetic polypeptide”
EVQLLESGGGLVQPGGSLKLSCAASGFTFNTYAMNWVRQAPGKGLEWVGRIRSKANNYATYYADSVKGRFTISRDDSKNILYLQMNSLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRAPGVPARFSGSLIGGKAALTIAGAQPEDEAEYYCALWYSNLWVFGGGTKLTVL

SEQ ID NO: 145 - protein, 250 aa, Artificial Sequence
  source: /note=“Description of Artificial Sequence Synthetic polypeptide”
EVQLLESGGGLVQPGGSLKLSCAASGFTFNTYAMNWVRQAPGKGLEWVGRIRSKANNYATYYADSVKGRFTISRDDSKNILYLQMNSLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGGKAALTLAGAQPEDEAEYYCALWYSNLWVFGGGTKLTVL

SEQ ID NO: 146 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCRSSTGAVTTSNYANWVQQKPGQAFRGLIGGTNKRAPGVPARFSGSLIGDKAALTITGAQPEDEAEYYCALWYSNLWVFGGGTKLTVL

SEQ ID NO: 147 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCRSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 148 - protein, 250 aa, Artificial Sequence
  source: /note=“Description of Artificial Sequence Synthetic polypeptide”
EVRLVESGGGLVQPGGSLKLSCAASGFTFNTYAMNWVRQAPGKGLEWVGRIRSKTNNYATYYADSVKDRFTISRDDAQNILYLQMNNLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLIGDKAALTIAGAQPEDEAEYYCALWYSNLWVFGGGTKLTVL

SEQ ID NO: 149 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKSNNYATYYAASVKDRFTISRDDSKNSLYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTNSNYANWVQQKPGQAFRGLIGGTNKRAPGTPARFSGSLLGGKAALTIAGVQPEDEAEYYCALWYSNLWVFGGGTKLTVL

SEQ ID NO: 150 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSKNTLYLQMNNLKTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPWTPARFSGSLIGDKAALTIAGAQPEDEAEYYCALWYSNLWVFGGGTKLTVL

SEQ ID NO: 151 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKSNNYATYYADSVKDRFTISRDDSKNILYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTIAGAQPEDEAEYYCALWYSNLWVFGGGTKLTVL

SEQ ID NO: 152 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKSNNYATYYADSVKDRFTISRDDSKNILYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGGKAALTLAGAQPEDEAEYYCALWYSNLWVFGGGTKLTVL

SEQ ID NO: 153 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDAQNILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGVPARFSGSLIGDKAALTLAGAQPEDEAEYYCALWYSNLWVFGGGTKLTVL

SEQ ID NO: 154 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSQSILYLQMNNLRAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGVPARFSGSLIGDKAALTITGAQPEDEAEYYCALWYSNLWVFGGGTKLTVL

SEQ ID NO: 155 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSQNILYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLIGGKAALTITGVQPEDEAEYYCALWYSNLWVFGGGTKLTVL

SEQ ID NO: 156 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADPVKGRFTISRDDAKNTLYLQMNSLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRAPGVPARFSGSLIGDKAALTITGAQPEDEAEYYCALWYSNLWVFGGGTKLTVL

SEQ ID NO: 157 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKSTLYLQMNNLRTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRAPGTPARFSGSLIGDKAALTITGAQPEDEAEYYCALWYSNLWVFGGGTKLTVL

SEQ ID NO: 158 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDAQNILYLQMNNLKTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRAPGTPARFSGSLLGDKAALTITGAQPEDEAEYYCALWYSNLWVFGGGTKLTVL

SEQ ID NO: 159 - protein, 250 aa, Artificial Sequence
  source: /note=“Description of Artificial Sequence Synthetic polypeptide”
EVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVL

SEQ ID NO: 160 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 161 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKGRFTISRDDAKSTLYLQMNSLRTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRAPGVPARFSGSLLGGKAALTLSGAQPEDEAEYYCALWYSNLWVFGGGTKLTVL

SEQ ID NO: 162 - protein, 250 aa, Artificial Sequence
  source: /note=“Description of Artificial Sequence Synthetic polypeptide”
EVQLLESGGGLVQPGGSLKLSCAASGFTFNTYAMNWVRQAPGKGLEWVARIRSKYNNYATYYADSVKDRFTISRDDSQNILYLQMNNLKTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 163 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDSKNTLYLQMNSLKTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 164 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVARIRSKYNNYATYYADSVKDRFTISRDDSKNILYLQMNSLRTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 165 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAQNILYLQMNNLKTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 166 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKGRFTISRDDSKSSLYLQMNSLKAEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAFRGLIGGTNKRAPGVPARFSGSLLGGKAALTLSGAQPEDEAEYYCALWYSNLWVFGGGTKLTVL

SEQ ID NO: 167 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSKNILYLQMNNLKAEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 168 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSQNILYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 169 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDAKNILYLQMNNLRTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 170 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKSILYLQMNNLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 171 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSKNILYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 172 - protein, 250 aa, Artificial Sequence
  source: /note=“Description of Artificial Sequence Synthetic polypeptide”
EVQLLESGGGLVQPGGSLKLSCAASGFTFNTYAMNWVRQAPGKGLEWVGRIRSKYNNYATYYADSVKGRFTISRDDSQNTLYLQMNNLKTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 173 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADPVKGRFTISRDDSKSTLYLQMNNLRTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 174 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKDRFTISRDDAKSTLYLQMNNLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 175 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKGRFTISRDDSQSTLYLQMNSLRAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 176 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVARIRSKYNNYATYYADSVKGRFTISRDDSKNTLYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 177 - protein, 250 aa, Artificial Sequence
  source: /note=“Description of Artificial Sequence Synthetic polypeptide”
EVQLLESGGGLVQPGGSLKLSCAASGFTFNTYAMNWVRQAPGKGLEWVGRIRSKYNNYATYYADSVKDRFTISRDDSKSTLYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 178 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVSRIRSKYNNYATYYADPVKDRFTISRDDAQNTLYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGDKAALTLSGAQPEDEAEYYCALWYSNLWVFGGGTKLTVL

SEQ ID NO: 179 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKGRFTISRDDSKNILYLQMNSLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 180 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDAKSILYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 181 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSQNTLYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAFRGLIGGTNKRAPGVPARFSGSLLGGKAALTLSGAQPEDEAEYYCALWYSNLWVFGGGTKLTVL

SEQ ID NO: 182 - protein, 250 aa, Artificial Sequence
  source: /note=“Description of Artificial Sequence Synthetic polypeptide”
EVQLVESGGGLVQPGGSLKLSCAASGFTFNTYAMNWVRQAPGKGLEWVGRIRSKYNNYATYYADSVKGRFTISRDDAKSTLYLQMNNLKTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 183 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDSQSILYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 184 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDAKNSLYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 185 - protein, 250 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADPVKDRFTISRDDSQNILYLQMNNLKAEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 186 - protein, 581 aa, Homo sapiens
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 187 - protein, 584 aa, Macaca fascicularis
MLAMPLKHYLLLLVGCQAWGAGLAYYGCPSECTCSRASQVECTGARIVAVPTPLPWNAMSLQILNTHITELSESPFLNISALIALRIEKNELSHIMPGAFRNLGSLRYLSLANNKLQVLPIGLFQGLDSLESLLLSSNQLVQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSIFMQLPQLNRLTLFGNSLKELSPGIFGPMPNLRELWLYDNHITSLPDNVFSNLRQLQVLILSRNQISFISPGAFNGLTELRELSLHTNALQDLDGNVFRMLANLQNISLQNNRLRQLPGNIFANVNGLMTIQLQNNQLENLPLGIFDHLGKLCELRLYDNPWRCDSDILPLRNWLLLNQPRLGTDTVPVCFSPANVRGQSLIIINVNVAVPSVHVPEVPSYPETSWYPDTSSYPDTTSISSTTELTSPVEDYTDLTTIQVTDDRSVWGMTQAQSGLAIAAIVIGIVALACSLAACIGCCCCKKRSQAVLMQMKAPNEC

SEQ ID NO: 188 - protein, 579 aa, Mus musculus
MPLKHYLLLLVSCQAWAAGLAYYGCPSECTCSRASQVECTGAQIVAMPSPLPWNAMSLQILNTHITELPEDKFLNISALIALKMEKNELANIMPGAFRNLGSLRHLSLANNKLKNLPVRLFQDVNNLETLLLSNNQLVQIQPAQFSQFSNLKELQLYGNNLEYIPEGVFDHLVGLTKLNLGNNGFTHLSPRVFQHLGNLQVLRLYENRLSDIPMGTFDALGNLQELALQENQIGTLSPGLFHNNRNLQRLYLSNNHISHLPPGIFMQLPHLNKLTLFGNSLKELSPGVFGPMPNLRELWLYNNHITSLPDNAFSHLNQLQVLILSHNQLSYISPGAFNGLTNLRELSLHTNALQDLDGNVFRSLANLRNVSLQNNRLRQLPGSIFANVNGLMTIQLQNNNLENLPLGIFDHLGNLCELRLYDNPWRCDSNILPLHDWLILNRARLGTDTLPVCSSPASVRGQSLVIINVNFPGPSVQGPETPEVSSYPDTSSYPDSTSISSTTEITRSTDDDYTDLNTIEPIDDRNTWGMTDAQSGLAIAAIVIGIIALACSLAACICCCCCKKRSQAVLMQMKAPNEC

SEQ ID NO: 189 - protein, 10 aa, Artificial Sequence
  source: /note=“Description of Artificial Sequence Synthetic peptide”
EPKSCDKTHT

SEQ ID NO: 190 - protein, 16 aa, Artificial Sequence
  source: /note=“Description of Artificial Sequence Synthetic peptide”
EPKSCDGGSGGSGGSG

SEQ ID NO: 191 - protein, 16 aa, Artificial Sequence
  source: /note=“Description of Artificial Sequence Synthetic peptide”
EPKSCDGGGGSGGGGS

SEQ ID NO: 192 - protein, 5 aa, Artificial Sequence
  source: /note=“Description of Artificial Sequence Synthetic peptide”
GGGGS

SEQ ID NO: 193 - protein, 10 aa, Artificial Sequence
  source: /note=“Description of Artificial Sequence Synthetic peptide”
GGGGSGGGGS

SEQ ID NO: 194 - protein, 214 aa, Artificial Sequence
  source: /note=“Description of Artificial Sequence Synthetic polypeptide”
DIQMTQSPSSLSASVGDRVTITCRASQDISNYLNWYQQKPGKAPKFLIYYTSRLHSGVPSRFSGSGSGTDYTLTISSLQPEDFATYYCQQGEALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 701 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 196 - protein, 701 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVL

SEQ ID NO: 197 - protein, 701 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSEVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVL

SEQ ID NO: 198 - protein, 708 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGSGGSEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 199 - protein, 708 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGSGGSEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVL

SEQ ID NO: 200 - protein, 708 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGSGGSEVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVL

SEQ ID NO: 201 - protein, 449 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 202 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 203 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 204 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 205 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVGRIRSKYNNYATYYADSVKGRFTISRDDSKNTLYLQMNNLKTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPWTPARFSGSLIGDKAALTIAGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 206 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 207 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 208 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 209 - protein, 705 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 210 - protein, 705 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 211 - protein, 705 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDEVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 212 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGSGGSGGSGEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 213 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGSGGSGGSGEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 214 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGSGGSGGSGEVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 215 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 216 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 217 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 218 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVGRIRSKYNNYATYYADSVKGRFTISRDDSKNTLYLQMNNLKTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPWTPARFSGSLIGDKAALTIAGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 220 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 221 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGSGGSGGSGEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 222 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGSGGSGGSGEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 715 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGSGGSGGSGEVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 224 - protein, 481 aa, Artificial Sequence
  source: /note=“Description of Artificial Sequence Synthetic polypeptide”
EVKLLESGGGLVQPGGSLKLSCAASGFTFNTYAMNWVRQAPGKGLEWVGRIRSKYNNYATYYADSVKGRFTISRDDAKSILYLQMNNLKTEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAFRGLIGGTNKRAPGTPARFSGSLIGGKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 481 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 226 - protein, 481 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 227 - protein, 481 aa, Artificial Sequence
  source: /note=“Description of Artificial Sequence Synthetic polypeptide”
EVKLVESGGGLVQPGGSLKLSCAASGFTFNTYAMNWVRQAPGKGLEWVGRIRSKYNNYATYYADSVKGRFTISRDDSKNTLYLQMNNLKTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPWTPARFSGSLIGDKAALTIAGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - protein, 481 aa, Artificial Sequence
  source: /note=“Description of Artificial Sequence Synthetic polypeptide”
EVKLVESGGGLVQPGGSLKLSCAASGFTFNTYAMNWVRQAPGKGLEWVARIRSKYNNYATYYADSVKDRFTISRDDAKNILYLQMNNLRTEDTAVYYCVRHGNFGNSYVSWFAYWGQGTLVTVSSGGGGSGGGGSGGGGSAQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 229 - protein, 15 aa, Artificial Sequence
  source: /note=“Description of Artificial Sequence Synthetic peptide”
GGGGSGGGGSGGGGS

SEQ ID NO: 230 - protein, 22 aa, Artificial Sequence
  source: /note=“Description of Artificial Sequence Synthetic peptide”
EPKSCDKTHTCPPCPAPEAAGA

SEQ ID NO: 231 - protein, 449 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 232 - protein, 481 aa, Artificial Sequence
  source: /note=“Description of Artificial Sequence Synthetic polypeptide”
EVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVLEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 233 - protein, 701 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSEVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVL

SEQ ID NO: 234 - protein, 708 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGSGGSEVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVL

SEQ ID NO: 235 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 236 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 237 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVLEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 238 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLLESGGGLVQPGGSLKLSCAASGFTFNTYAMNWVRQAPGKGLEWVGRIRSKANNYATYYADSVKGRFTISRDDSKNTLYLQMNNLKAEDTAVYYCVRHGNFGNSYVSWFAYWGQGTLVTVSSGGGGSGGGGSGGGGSAEAVVTQEPSLTVSPGGTVTLTCGSSTGAVTTSNYANWVQQKPGQAPRGLIGGTNKRAPGTPARFSGSLLGDKAALTITGAQPEDEAEYYCALWYSNLWVFGGGTKLTVLEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 239 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVARIRSKYNNYATYYADSVKDRFTISRDDAKNTLYLQMNNLRTEDTAVYYCVRHGNFGNSYVSWFAYWGQGTLVTVSSGGGGSGGGGSGGGGSAQAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 240 - protein, 709 aa, Artificial Sequence
  source: /note=“Description of Artificial Sequence Synthetic polypeptide”
QVQLVQSGAEVKKPGASVKVSCKASGYKFTSYWIEWVRQAPGQGLEWIGEILPGSDTTNYNEKFKDRVTFTSDTSTSTVYMELSSLRSEDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTEVKLVESGGGLVQPGGSLKLSCAASGFTFNTYAMNWVRQAPGKGLEWVGRIRSKYNNYATYYADSVKDRFTISRDDSQNILYLQMNNLRTEDTAVYYCVRHGNFGNSYVSWFAYWGQGTLVTVSSGGGGSGGGGSGGGGSAQAFVTQEPSLTVSPGGTVTLTCGSSTGAVTTSNYANWVQQKPGQAPRGLIGGTNKRAPGVPARFSGSLIGDKAALTITGAQPEDEAEYYCALWYSNLWVFGGGTKLTVLEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 241 - protein, 10 aa, Artificial Sequence
  source: /note=“Description of Artificial Sequence Synthetic peptide”
EPKSSDKTHT

SEQ ID NO: 242 - protein, 22 aa, Artificial Sequence
  source: /note=“Description of Artificial Sequence Synthetic peptide”
EPKSSDKTHTCPPCPAPEAAGA

SEQ ID NO: 243 - protein, 109 aa, Artificial Sequence
  source: /note=“Description of Artificial Sequence Synthetic polypeptide”
QAVVTQEPSLTVSPGGTVTLTCRSSTGAVTTSNYANWVQQKPGQAPRGLIGGTNKRAPGTPARFSGSLIGDKAALTITGVQPEDEAEYYCALWYSNLWVFGGGTKLTVL

SEQ ID NO: 244 - protein, 16 aa, Artificial Sequence
  source: /note=“Description of Artificial Sequence Synthetic peptide”
GGGGSGGGGSGGGGSA